(19) United States
(12) Patent Application Publication
(10) Pub. No.: US 2011/0091501 A1
(43) Pub. Date: Apr. 21, 2011

(54) Recombinant Rhinovirus Vectors

(75) Inventors: Kirill Kalnin, Pelham, NH (US); Yanhua Yan, Westford, MA (US); Maryann Giel-Moloney, Brighton, MA (US); Harold Kleanthous, Westford, MA (US)

(73) Assignee: SANOFI PASTEUR BIOLOGICS CO., Cambridge, MA (US)

(21) Appl. No.: 12/934,095

(22) PCT Filed: Mar. 27, 2009
(86) PCT No.: PCT/US2009/001941

§ 371 Date: Dec. 23, 2010

Related U.S. Application Data
(60) Provisional application No. 61/072,036, filed on Mar. 27, 2008.

(51) Int. Cl. A61K 39/29; C12N 15/63; A61K 31/7088; A61K 38/16; C07H 21/02; C07K 2/00; C12N 15/64; A61P 31/16; A61P 37/04

(52) U.S. Cl. 424/227.1; 435/320.1; 514/44 R; 514/21.2; 536/23.72; 530/300; 435/91.4

(57) ABSTRACT

The invention provides rhinovirus vectors, which can be used in the delivery of immunogens, such as influenza virus immunogens, and corresponding compositions and methods.

### BACKGROUND OF THE INVENTION

An influenza pandemic occurs when a new influenza virus subtype appears, against which the global population has little or no immunity. During the 20^{th} century, influenza pandemics caused millions of deaths, social disruption, and profound economic losses worldwide. Influenza experts agree that another pandemic is likely to happen, but it is unknown when. The level of global preparedness at the moment when a pandemic strikes will determine the public health and economic impacts of the disease. As of today, the World Health Organization (WHO) estimates that there will be at least several hundred million outpatient visits, more than 25 million hospital admissions, and several million deaths globally, within a very short period. These concerns were highlighted in 2003, when the avian H5N1 virus reached epizootic levels in domestic fowl in a number of Asian countries, and then spread to Europe and Africa. Fortunately, its transmission to humans has so far been limited, with 246 documented infections, which were associated with high mortality accounting for 144 deaths, as reported on Sep. 14, 2006 (World Health Organization (WHO) Web site).

Conventional influenza vaccines are designed to elicit neutralizing antibody responses against influenza virus hemagglutinin protein (HA). Due to the constant antigenic drift in the HA protein, the vaccine composition must be changed each year to match anticipated circulating viral strains. Such a vaccine approach is unacceptable in the face of a pandemic, because of the long time required for the isolation and identification of a pandemic strain, and construction and manufacture of an appropriate vaccine. A more effective approach to control or prevention of an influenza pandemic contemplates development of a “universal” vaccine capable of eliciting protective immunity against recently identified, highly conserved influenza virus immunological determinants. Such a vaccine should provide broad protection across influenza A virus strains. Further, such a vaccine could be manufactured throughout the year, stockpiled, and/or administered throughout the year.

The 19-25 amino acid sequence surrounding the proteolytic cleavage site of hemagglutinin (HA) is a conserved influenza A virus epitope (Bianchi et al., J. Virol. 79:7380-7388, 2005; Mundy et al., Science 303:1870-1873, 2004). The mature influenza virus HA is composed of two subunits, HA₁ and HA₂, which are derived from the precursor HA₀ by proteolytic cleavage (Chen et al., Cell 95:409-417, 1998; Skehel et al., Proc. Natl. Acad. Sci. U.S.A. 72:93-97, 1975). Based on crystallographic data (Gamblin et al., Science 303:1838-1842, 2004; Stevens et al., Science 303:1866-1870, 2004), it was determined that the cleavage site forms an extended, solvent-exposed loop. Upon cleavage, the newly formed N-terminus of HA₂ hosts the fusion peptide, which mediates fusion of viral and cellular membranes. HA₀ cleavage is crucial for virus infectivity (Klenk et al., Virology 68:426-439, 1975; Klenk et al., Virology 68:426-439, 1975) and pathogenicity (Klenk et al., Trends Microbiol. 2:39-43, 1994; Steinhauer, Virology 258:1-20, 1999). Because of functional constraints, the epitope is extremely well conserved, and thus may elicit a broad cross-protective response (Bianchi et al., J. Virol. 79:7380-7388, 2005).

The HA₀ peptide of influenza B virus conjugated to outer membrane protein complex of Neisseria meningitides elicits a protective immune response in Balb/c mice (Bianchi et al., J. Virol. 79:7380-7388, 2005). An alignment of human and avian influenza A and influenza B HA₀ sequences is shown below. The conserved nature of this region was confirmed in a study of more than 700 Indonesian and Vietnamese influenza A human and avian virus strains (Smith et al., Virology 350:258-268, 2006). Some mutations were observed, but they occurred mostly upstream from the cleavage site (indicated by the arrow in the alignment below) (Smith et al., Virology 350:258-268, 2006). Systematic alanine-scanning mutagenesis of the HA₀ peptide of influenza B elucidated three residues, R6, F9, and F15 (boxed in the alignment), as the most critical residues for binding of three protective HA₀-specific monoclonal antibodies (Bianchi et al., J. Virol. 79:7380-7388, 2005). These residues are conserved among all avian and human influenza A and influenza B strains (below; SEQ ID NOs:1-8).

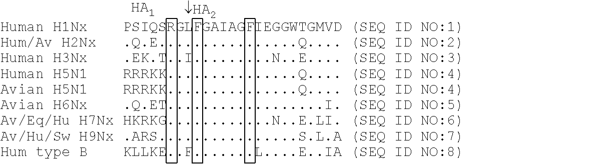

The influenza virus matrix protein M2 has been demonstrated to serve as an effective target for vaccine development (DeFilette et al., Virology 337:149-161, 2005). M2 is a 97-amino-acid transmembrane protein of influenza type A virus (Lamb et al., Proc. Natl. Acad. Sci. U.S.A. 78:4170-4174, 1981; Lamb et al., Cell 40:627-633, 1985). The mature protein forms homotetramers (Holsinger et al., Virology 183:32-43, 1991; Sugrue et al., Virology 180:617-624, 1991) that have pH-inducible ion channel activity (Pinto et al., Cell 69:517-528, 1992; Sugrue et al., Virology 180:617-624, 1991). M2-tetramers are expressed at high density in the plasma membrane of infected cells and are also incorporated at low frequency into the membranes of mature virus particles (Takeda et al., Proc. Natl. Acad. Sci. U.S.A. 100:14610-14617, 2003; Zebedee et al., J. Virol. 62:2762-2772, 1998). The M2 N-terminal 24-amino-acid ectodomain (M2e) is highly conserved among type A influenza viruses (Fiers et al., Virus Res. 103:173-176, 2004). The high degree of conservation of M2e can be explained by constraints resulting from its genetic relationship to M1, the most conserved protein of the virus (Ito et al., J. Virol. 65:5491-5498, 1991), and the absence of M2e-specific antibodies during natural infection (Black et al., J. Gen. Virol. 74 (Pt 1):143-146, 1993).

As shown in the alignment below, obtained using sequences from the NCBI influenza database, avian H5N1 influenza virus M2e appears to be evolving toward the consensus sequence found in typical human H1, H2, and H3 viruses, suggesting that broad protection, including from new avian viruses, using the “human” influenza M2e epitope may be a possibility (below, SEQ ID NOs:9-12).

| | |
|---|---|
| Human H1N1 | |
| MSLLTEVETPIRNEWGCRCNDSSD | (SEQ ID NO: 9) |
| Human H5N1 2001-2006 | |
| MSLLTEVETPTRNEWECRCSDSSD | (SEQ ID NO: 10) |
| Human H5N1 1997-2000 | |
| MSLLTEVETLTRNGWGCRCSDSSD | (SEQ ID NO: 11) |
| Avian H5N1 1983-1998 | |
| MSLLTEVETLTRNGWGCRCSDSSD | (SEQ ID NO: 12) |

The phenomenon of evolution of the H5N1 M2e towards the H1N1 M2e sequence was recently reported based on the analysis of sequences of 800 H5H1 strains isolated from humans and birds in Indonesia and Vietnam (Smith et al., Virology 350:258-268, 2006). The evolved avian M2e peptide EVETPTRN (SEQ ID NO:13), but not its “predecessor” EVETLTRN (SEQ ID NO:14), was efficiently recognized by an anti-human M2e monoclonal antibody (Mab) (Liu et al., Microbes Infect. 7:171-177, 2005). This is important, because some “bird-flu-like” changes have been shown previously to reduce the effectiveness of protection provided by human M2e specific monoclonal antibodies. Interestingly, some “bird-flu-like” amino acid changes in M2e reduced pathogenicity of human H1N1 viruses in mice (Zharikova et al., J. Virol. 79:6644-6654, 2005).

The WHO has emphasized the possibility of a “simultaneous occurrence of events with pandemic potential with different threat levels in different countries, as was the case in 2004 with poultry outbreaks of H7N3 in Canada and H5N1 in Asia.” As is shown in the alignment below, M2e H7N7 differs at only one amino acid from the “humanized” variant of H5N1. The H7N7 subtype has demonstrated the ability to be transmissible between species (Koopmans et al., Lancet 363:587-593, 2004) and can be lethal for people (Fouchier et al., Proc. Natl. Acad. Sci. U.S.A. 101:1356-1361, 2004). The other strains (H9N2) were also shown to be able to infect poultry and spread to people (Cameron et al., Virology 278:36-41, 2000; Li et al., J. Virol. 77:6988-6994, 2003; Wong et al., Chest 129:156-168, 2006)(below, SEQ ID NOs:9 and 15-18).

| | |
|---|---|
| Human H1N1 | |
| MSLLTEVETPIRNEWGCRCNDSSD | (SEQ ID NO: 9) |
| Avian/Equine H7N7 | |
| MSLLTEVETPTRNGWECRCSDSSD | (SEQ ID NO: 15) |
| Avian H9Nx 1966-1996 | |
| MSLLTEVETPTRNGWECKCSDSSD | (SEQ ID NO: 16) |
| Avian H9Nx 1997-2004 | |
| MSLLTEVETHTRNGWGCRCSDSSD | (SEQ ID NO: 17) |
| Human H9N2 1999-2003 | |
| MSLLTEVETLTRNGWECKCSDSSD | (SEQ ID NO: 18) |

M2e-based recombinant protein vaccines have been shown to elicit protective immune responses against both homologous and heterologous influenza A virus challenges (Fiers et al., Virus Res. 103:173-176, 2004; Slepushkin et al., Vaccine 13:1399-1402, 1995). More recent studies using an M2e peptide conjugated to keyhole limpet hemocyanin and N. meningitides outer membrane protein illustrated good immune responses not only in mice, but also in ferrets and rhesus monkeys (Fan et al., Vaccine 22:2993-3003, 2004). Protection against H1, H5, H6, and H9 influenza A viruses with a liposomal M2e vaccine was demonstrated in mice (Fan et al., Vaccine 22:2993-3003, 2004).

Effective delivery systems for influenza immunogens are important for the development of vaccines against influenza virus infection, such as pandemic vaccines.

### SUMMARY OF THE INVENTION

The invention provides rhinovirus vectors (live or inactivated) including influenza virus HA₀ immunogens. Such vectors can be nonpathogenic in humans, such as Human Rhinovirus 14 (HRV14). In addition to HA₀ sequences, the rhinovirus vectors can, optionally, include one or more M2e peptides. These peptides (HA₀ and/or M2e) can be inserted, for example, at the site of a neutralizing immunogen selected from the group consisting of Neutralizing Immunogen I (NimI), Neutralizing Immunogen II (NimII) (e.g., between amino acids 158 and 160), Neutralizing Immunogen III (NimIII), and Neutralizing Immunogen IV (NimIV), or at more than one of these sites. Further, the peptides may, optionally, be flanked by linker sequences on one or both ends.

The invention also provides pharmaceutical or immunogenic compositions that include the rhinovirus vectors described herein and a pharmaceutically acceptable carrier or diluent. Optionally, such compositions can include one or more adjuvants, and/or one or more additional active ingredients (e.g., a Hepatitis B core protein fused with HA₀ and/or M2e sequences, and/or a rhinovirus vector including an HA₀ peptide and a rhinovirus vector including an M2e peptide).

Also included in the invention are methods of inducing an immune response to an influenza virus in a subject (e.g., a human subject), in which a pharmaceutical composition as described herein is administered to the subject. The subject may not have but be at risk of developing influenza virus infection, or the subject may have influenza virus infection. The composition can be administered by, for example, the intranasal route. The invention also includes use of the vectors and compositions described herein in methods for inducing immune responses, as described herein, and use of the vectors and compositions in the preparation of medicaments, for uses such as those described herein.

The invention also provides methods of making pharmaceutical compositions, as described herein, which involve admixing the rhinovirus vectors, as described herein, with a pharmaceutically acceptable carrier or diluent (and, optionally, additional components, as described herein).

Further, the invention provides nucleic acid molecules encoding or corresponding to the genomes of the rhinovirus vectors described herein (in DNA or RNA form).

The invention further includes NimII peptides including one or more inserted influenza virus HA₀ immunogens, as described herein.

The invention provides methods of generating rhinovirus vectors (e.g., HRV14 vectors) including one or more influenza virus HA₀ immunogens (and, optionally other immunogens, such as M2e immunogens). These methods include the steps of: (i) generating a library of recombinant rhinovirus vectors based on an infectious cDNA clone that contains inserted influenza virus HA₀ immunogen sequences, and (ii) selecting from the library recombinant viruses that (a) maintain inserted sequences upon passage, and (b) are neutralized with antibodies against the inserted sequence. In these methods, inserted influenza immunogen sequences can be inserted at a position selected from the group consisting of NimI, NimII, NimIII, and NimIV. Further, the inserted sequence(s) may, optionally, be flanked on one or both ends with random linker sequences.

Also included in the invention are methods for cultivating rhinovirus vectors as described herein, which involve passaging the vectors in cells, such as HeLa or MRC-5 cells.

Further, the invention includes rhinovirus vectors as described herein comprising one or more immunogens, as described herein.

The invention provides several advantages. For example, in the case of the live vectors of the invention, use of such live vectors system to deliver immunogens such as HA₀ provides advantages including: (i) the ability to elicit very strong and long-lasting antibody responses with as little as a single dose of vaccine, and (ii) greater scalability of manufacturing (i.e., more doses at a lower cost) when compared with subunit or killed vaccines. Thus, in a pandemic situation, many more people could be immunized in a relatively short period of time with a live vaccine. In addition, the HRV vectors of the invention can be delivered intranasally, resulting in both systemic and mucosal immune responses. Use of HRV14 provides additional advantages, as it is nonpathogenic and is infrequently observed in human populations (Andries et al., J. Virol. 64:1117-1123, 1990; Lee et al., Virus Genes 9:177-181, 1995), which reduces the probability of preexisting anti-vector immunity in vaccine recipient. Further, the amount of HRV needed to infect humans is very small (one tissue culture infectious dose (TCID₅₀) (Savolainen-Kopra, “Molecular Epidemiology of Human Rhinoviruses,” Publications of the National Public Health Institute February 2006, Helsinki, Finland, 2006), which is a favorable feature in terms of cost-effectiveness of HRV-based vaccine manufacturing.

Other features and advantages of the invention will be apparent from the following Detailed Description, the Drawings, and the Claims.

### BRIEF DESCRIPTION OF THE DRAWINGS

FIG. 1 is a schematic representation of a virus particle (upper panel) and genome (lower panel) of HRV14. The human rhinovirus 14 (HRV14) capsid exhibits a pseudo-T=3 (P=3) isochedral symmetry and consists of 60 copies of viral proteins VP1, VP2, VP3, and VP4, with VP4 at the RNA-capsid interface (Rossmann et al., Nature 317:145-153, 1985). VP 1-3 proteins form a canyon containing a receptor-binding site for a cellular receptor, intracellular adhesion molecule 1 (ICAM-1) (Colonno et al., J. Virol. 63:36-42, 1989). Three major neutralizing immunogenic (Nim) sites, NimI (AB), NimII, and NimIII were identified on the surface of the canyon rim as binding sites for neutralizing antibodies (Sherry et al., J. Virol. 57:246-257, 1986). The reconstruction of the HRV14 particle was created in Chimera program on the basis of HRV14 crystal structure with NimI-specific mAb17 (protein databank database #1RVF).

FIG. 2 is described as follows: (A) HRV14-M2e constructs created in this study (SEQ ID NOs:19-21). A derivative of the HRV14 cDNA clone, plasmid pWR1, was used for constructions of M2e-insertion mutants. (B) Plaques produced by HRV14-NimII-XXX17AA (Arnold et al., J. Mol. Biol. 177:417-430, 1984) and HRV14-NimII-XXX23AA (Arnold et al., US 2006/0088549 A1) virus libraries, as well as wild type HRV14 derived from pWR1. Construct #1 did not yield plaques, as discussed in the text and supported by additional data (FIGS. 3 and 4), indicating that the random linker strategy is an effective means of engineering novel epitopes in HRV. Panel (C) shows HRV14-M2e (17AA), HRV14-HA₀ (19AA), and HRV14-M2e16HA₀12 constructs, according to the invention (SEQ ID NOs:22-24).

FIG. 3 shows the stability of the M2e insert in different HRV14-M2e constructs. The insert-containing fragments were RT-PCR amplified with pairs of primers, P1-up100Fw, VP1-dwn200Rv (green), or 14FAfIII-1730Rv (red), resulting in “PCR B” (green) or “PCR A” (red) DNA fragments, respectively. These fragments were digested with XhoI. Agarose gel electrophoresis results for HRV14-M2e chimera at passages 2, 3, and 4, and for HRV14-NimII-XXX17AA and HRV14-NimII-XXX17AA virus libraries at passage 4, are shown. The two cleaved fragments (indicated by arrows) represent insert-containing virus.

FIG. 4 shows possible steric interference of the 23 amino acid M2e insert in the NimII site with the receptor-binding domain of HRV14. The insert without linkers could stretch out from NimII and almost reach the opposite side of the canyon (i.e., at the NimI site), as shown in the picture. That barrier could effectively block receptor entrance into the canyon. An N-terminal linker can change position of the insert (direction is shown by arrow) and open access to the canyon. This molecular model of VP1-VP4 subunit of HRV14-NimII-M2e (23 amino acids) was created in Accelrys Discovery Studio (Accelrys Software, Inc). This illustrates our ability to engineer novel epitopes into HRV14 due to the available structural data and modeling software.

FIG. 5 shows plaque reduction neutralization test (PRNT) of HRV14, the HRV14-NimII-XXX23AA library, and the HRV14-NimII-XXX17AA library with anti-M2e Mab 14C2 (Abcam, Inc; Cat# ab5416). The results demonstrate efficient neutralization of both libraries, but not of the vector virus (HRV14). The purity of both libraries (absence of WT contamination) is also evident from these results.

FIG. 6 shows M2e-specific IgG antibody response (pooled samples) in immunized mice prior to challenge. End point titers (ETs) are shown after relevant group titles. Time of corresponding immunizations is shown in parentheses (d0 and d21 stand for day 0 and day 21, respectively).

FIG. 7 shows HRV14-specific IgG antibody response (pooled samples) in immunized mice prior to challenge: (A) groups immunized with 1, 2, or 3 doses of HRV14-M2e (17AA) virus; and (B) groups immunized with one or two doses of parental HRV14 virus.

FIG. 8 shows individual M2e-specific IgG antibody responses of immunized mice.

FIG. 9 shows M2e-specific antibody isotypes IgG1 and IgG2a in mice immunized as described in Table 4: (A) IgG1 ELISA (group pooled samples); (B) IgG2a ELISA (group pooled samples); (C) Titles for Figs. A and B; (D) Level of M2-e-specific IgG1 (dots) and IgG2a (diamonds) in individual sera samples (dilution 1:2,700) of group 4 (red; first and third sets of data) and group 7 (green; second and fourth sets of data) mice (see Table 4).

FIG. 10 shows M2e-specific antibodies of IgG2b isotype in mice immunized as described in Table 4. (A) ELISA with M2e peptide (group pooled samples); (B) Individual sera samples (dilution 1:2,700) of group 4 (red) and group 7 (green) mice (see Table 1) tested in ELISA against M2e-specific peptide.

FIG. 11 shows M2e-specific antibodies of IgG1, IgG2a, and IgG2b isotypes in mice immunized as described in Table 4 (upper panel).

FIG. 12 shows survival rates of all groups 28 days after challenge with the PR8 Influenza A strain.

FIG. 13 shows morbidity of all groups 28 days after challenge with PR8 Influenza A strain (FIG. 13A); Individual body weights within group 4 (FIG. 13B) and group 7 (FIG. 13C).

FIG. 14 shows M2e (A-D) and HA₀ (E)-specific IgG antibody response (pooled samples) in immunized mice prior to challenge (for groups see Table 5).

FIG. 15 shows the morbidity (B; percentage of bodyweight) and mortality (A; survive %) of all groups during 21 days after mortal challenge with PR8 Influenza A strain.

FIG. 16 shows the results of plaque reduction neutralization test (PRNT) of HRV14 and HRV6 with mouse anti-HRV14-NimIV^{HRV6} serum. These data served as a proof of immunodominance of NimIV^{HRV6} in the background of HRV14 capsid, suggesting a novel site for insertion of foreign epitopes.

FIG. 17 is a schematic illustration of the insertions sites in the virion proteins of HRV14. M2e or HA₀ is introduced in the indicated positions of NimI, NimII, NimIII, and NimIV. XXXM2e signifies M2e libraries described herein (SEQ ID NOs:25-28).

FIG. 18 provides sequence information for Human Rhinovirus 14 (HRV14). The encoded amino acid sequence (SEQ ID NO:30) is obtained by translation of nucleotides 629-7168 of indicated nucleic acid sequence (SEQ ID NO:29).

FIG. 19 provides a plasmid map and the sequence information for the 19 amino acid HA sequence inserted into the NimII site of HRV14 (SEQ ID NO:77) and the full sequence of CMVHRV14MGM19aaHAGQ (SEQ ID NO:78).

FIG. 20 provides a plasmid map and the sequence information for the P1 region amino acid sequence of HRV14-M2e17aa (SEQ ID NO:79) and the plasmid sequence of M2e17aa in NimII HRV14 (SEQ ID NO:80).

FIG. 21 provides a plasmid map and the sequence information for the M2e 23 amino acid (mutated) sequence (SEQ ID NO: 81), the P1 region amino acid sequence of HRV14-M2e23aa (SEQ ID NO:82), and the plasmid sequence of M2e23aa in NimII HRV14 (SEQ ID NO:83).

FIG. 22 provides a plasmid map and the sequence information for the P1 region amino acid sequence of HRV14-M2e16aa-HA012aa (SEQ ID NO:84) and the plasmid sequence of HA012-M2e16 in NimII HRV14 (SEQ ID NO:85).

FIG. 23 provides a construct map and the sequence information for the VP4-VP1 (structural region) of HRV14-M2e (17AA) chimera (SEQ ID NO:86) and the VP4-VP1 (structural region) of HRV14-M2e (23AA) chimera (SEQ ID NO:87).

### DETAILED DESCRIPTION

The invention provides universal (pandemic) influenza vaccines, which are based on the use of human rhinoviruses (HRV) as vectors for efficient delivery and presentation of influenza virus determinants. As described further below, the proteolytic cleavage site of influenza virus hemagglutinin (HA) (HA₀) and the extracellular domain of the influenza virus matrix protein 2 (M2e) are two epitopes that can be included in a universal influenza (influenza A) vaccine, according to the invention. The vaccines of the invention thus include vectors containing one or more HA₀-based immunogen(s), and can optionally be used in combination with an M2e-based immunogen, which can be in the same composition as the HA₀-based immunogen, linked to the HA₀-based immunogen (directly or indirectly, e.g., by a linker), or in a separate composition from the HA₀-based immunogen. The vaccine compositions of the invention can be used in methods to prevent or treat influenza virus infection, including in the context of an influenza pandemic. The invention also includes vectors as described herein including other immunogens, as described further below. The vectors, vaccines, compositions, and methods of the invention are described further, as follows.

### HRV Vectors

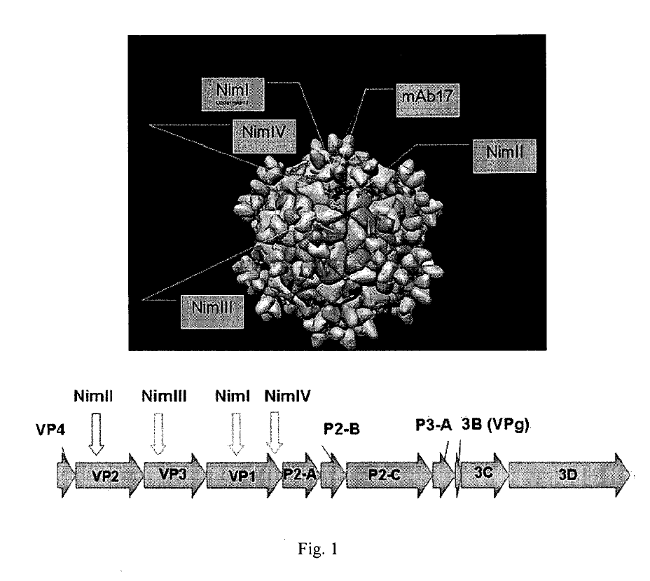

The vectors of the invention are based on human rhinoviruses, such as the non-pathogenic serotype human rhinovirus 14 (HRV14). The HRV14 virus particle and genome structure are schematically illustrated in FIG. 1, which shows virus structural proteins (VP1, VP2, VP3, and VP4), the non-structural proteins (P2-A, P2-B, P-2C, P3-A, 3B(VPg), 3C, and 3D), as well as the locations of major neutralizing immunogenic sites in HRV14 (Nims: NimI, NimII, NimIII, and NimIV).

An example of a molecular clone of HRV14 that can be used in the invention is pWR3.26 (American Type Culture Collection: ATCC® Number: VRMC-7™). This clone is described in further detail below, as well as by Lee et al., J. Virology 67(4):2110-2122, 1993 (also see SEQ ID NOs:29 and 30). Additional sources of HRV14 can also be used in the invention (e.g., ATCC Accession No. VR284; also see GenBank Accession Nos. L05355 (Jun. 11, 1993) and K02121 (Jan. 2, 2001) and other listed versions thereof; Stanway et al., Nucleic Acids Res. 12(20):7859-7875, 1984; and Callahan et al., Proc. Natl. Acad. Sci. U.S.A. 82(3):732-736, 1985). In addition to HRV14, other human rhinovirus serotypes can be used in the invention. As is known in the art, there are more than 100 such serotypes, any of which can be used upon the derivation of an infectious clone, such as in the same manner as for HRV14. Thus, although described herein with respect to HRV14, the invention also applies to other rhinovirus serotypes, as well as variants thereof (e.g., variants including sequence differences that are naturally occurring or artificial, which do not substantially affect virus properties or which provide attenuation; and also variants including one or more (e.g., 1-100, 2-75, 5-50, or 10-35) conservative amino acid substitutions).

Antigen sequences can be inserted into HRV vectors, according to the invention, at different sites, as described further below. In one example, the sequences are inserted into the NimII site of a serotype such as HRV14. NimII (Neutralizing Immunogen II) is an immunodominant region in HRV14 that includes amino acid 210 of VP1 and amino acids 156, 158, 159, 161, and 162 of VP2 (Savolainen-Kopra, “Molecular Epidemiology of Human Rhinoviruses,” Publications of the National Public Health Institute February 2006, Helsinki, Finland, 2006). In specific examples described below, the sequences are inserted between amino acids 158 and 160, or 158 and 162 of VP2. Insertions can be made at other sites within the NimII site as well. For example, the insertion can be made at any of positions 156, 158, 159, 161, or 162 of VP2, or at position 210 of VP1, or combinations thereof. References to positions of insertions herein generally indicate insertions carboxy-terminal to the indicated amino acid, unless otherwise indicated, and can also be made in connection with deletions as described herein.

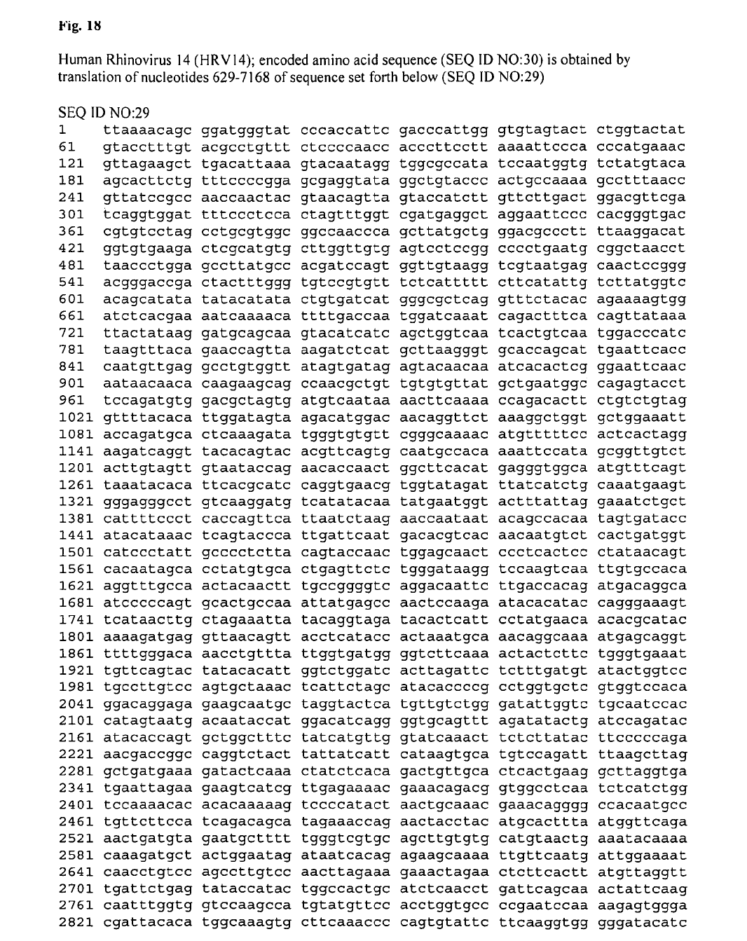

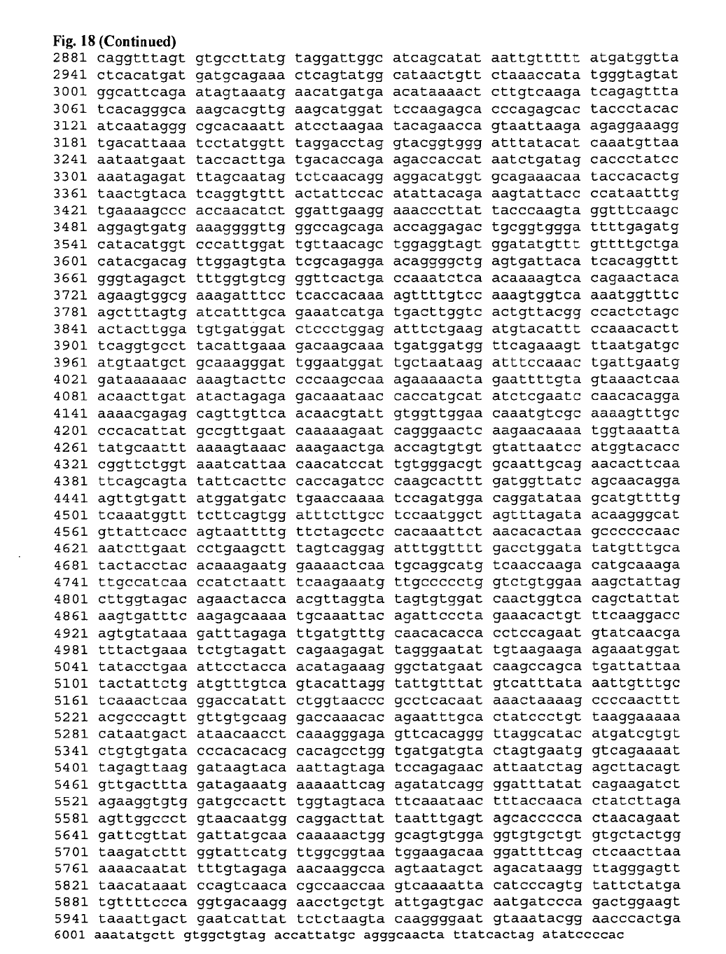

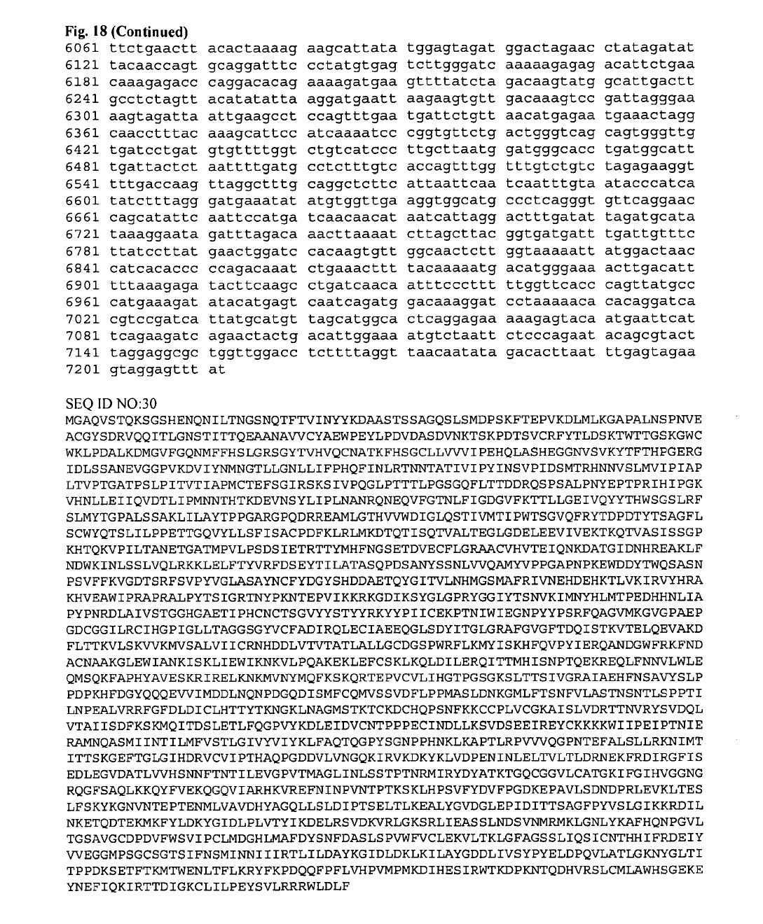

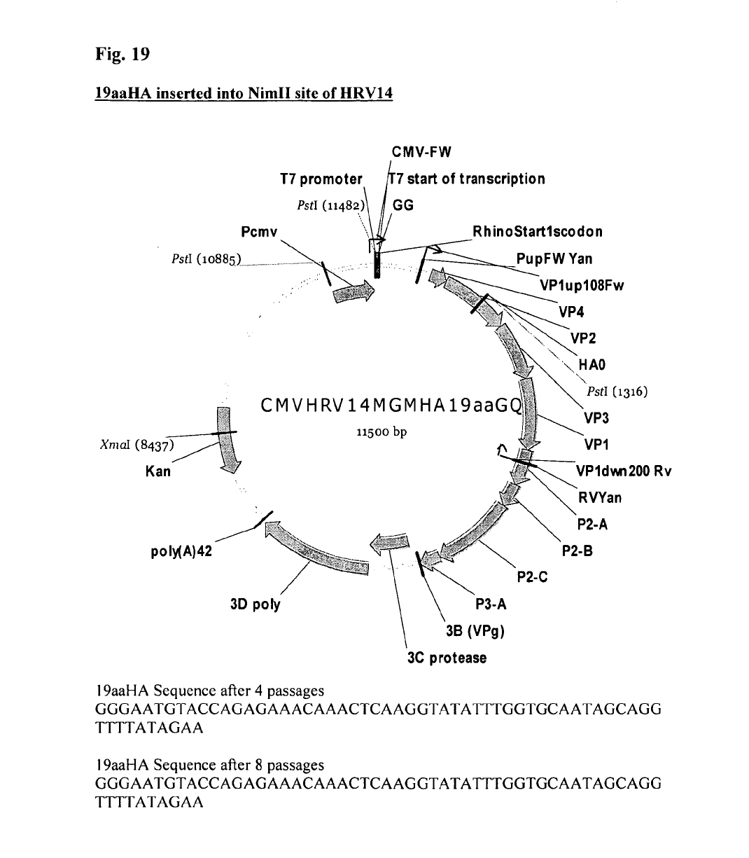

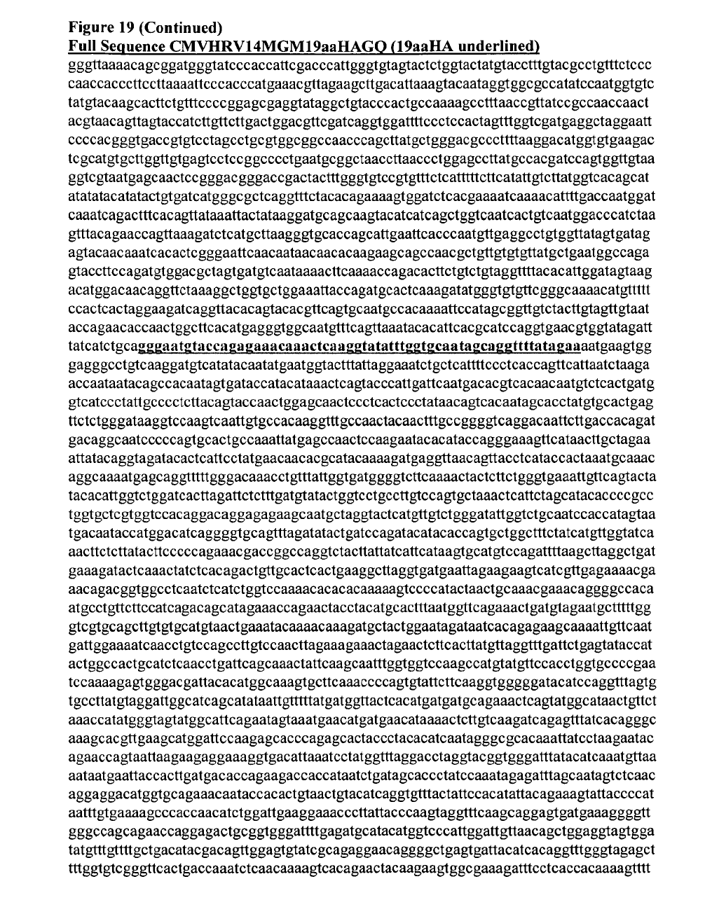

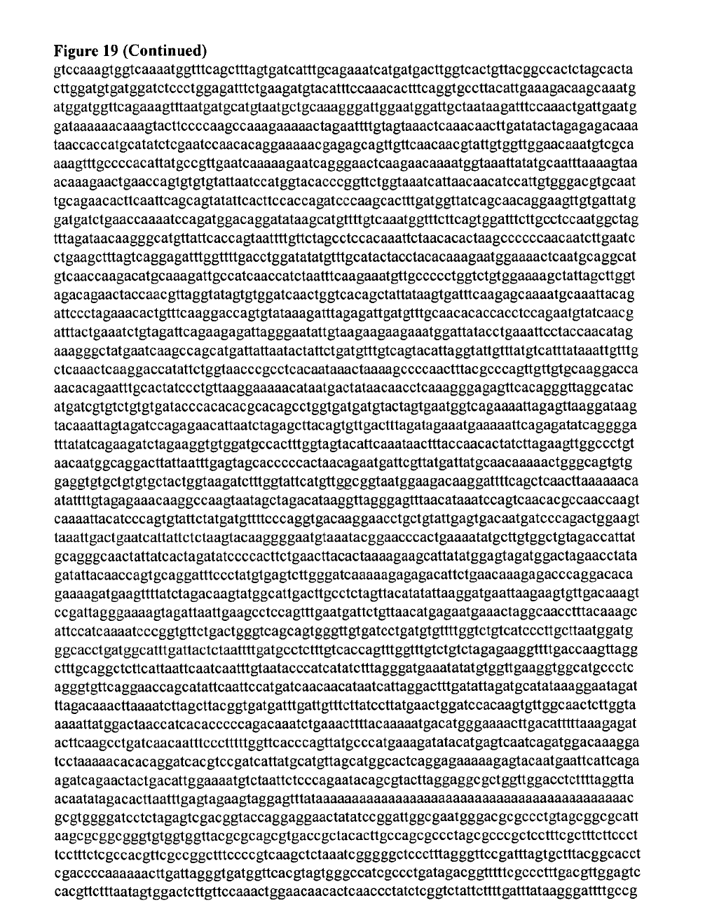

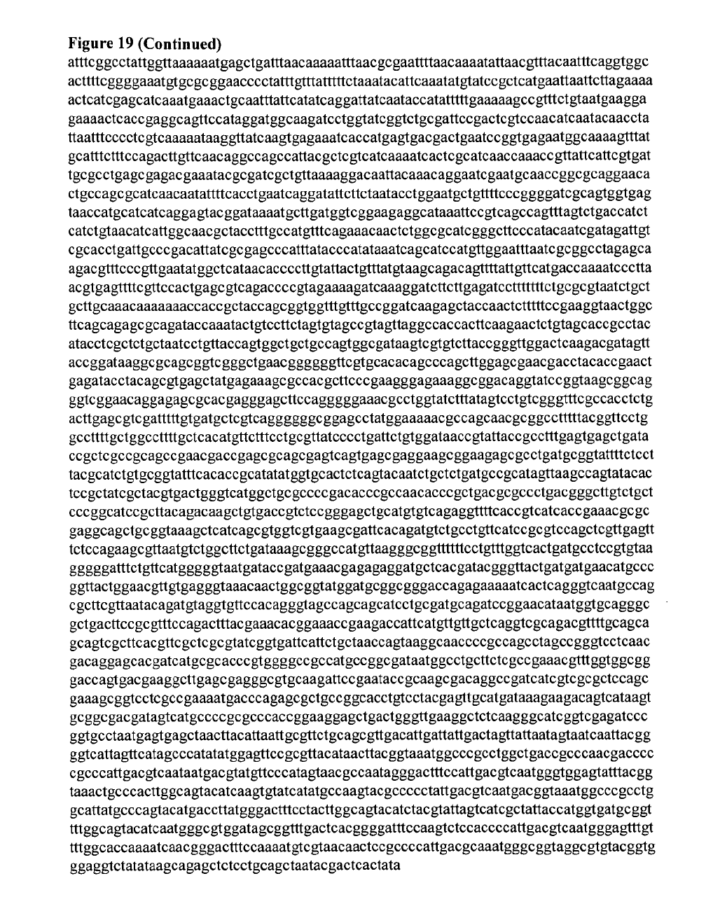

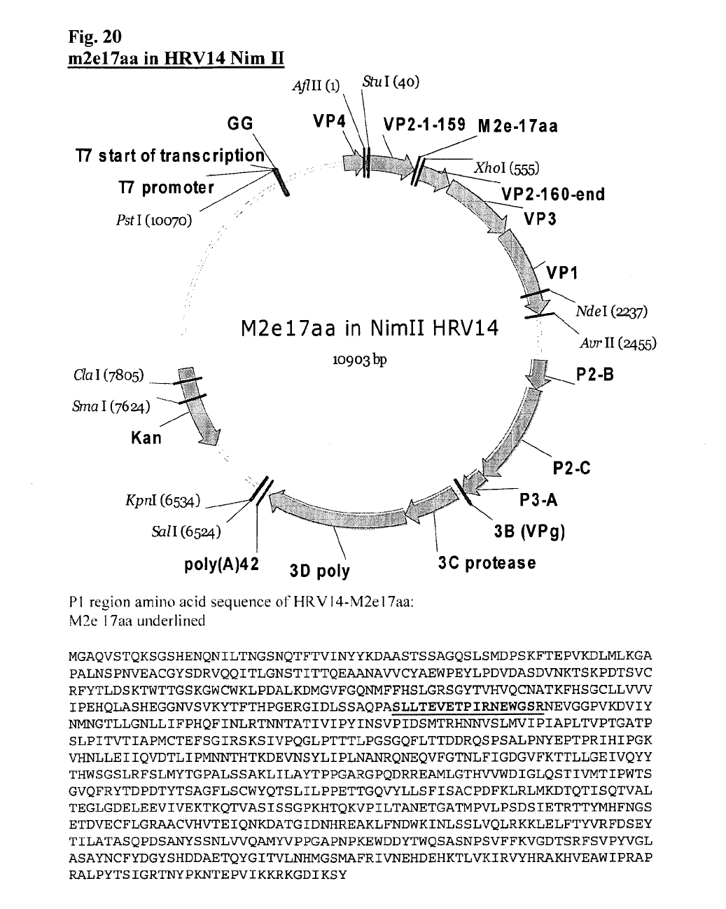

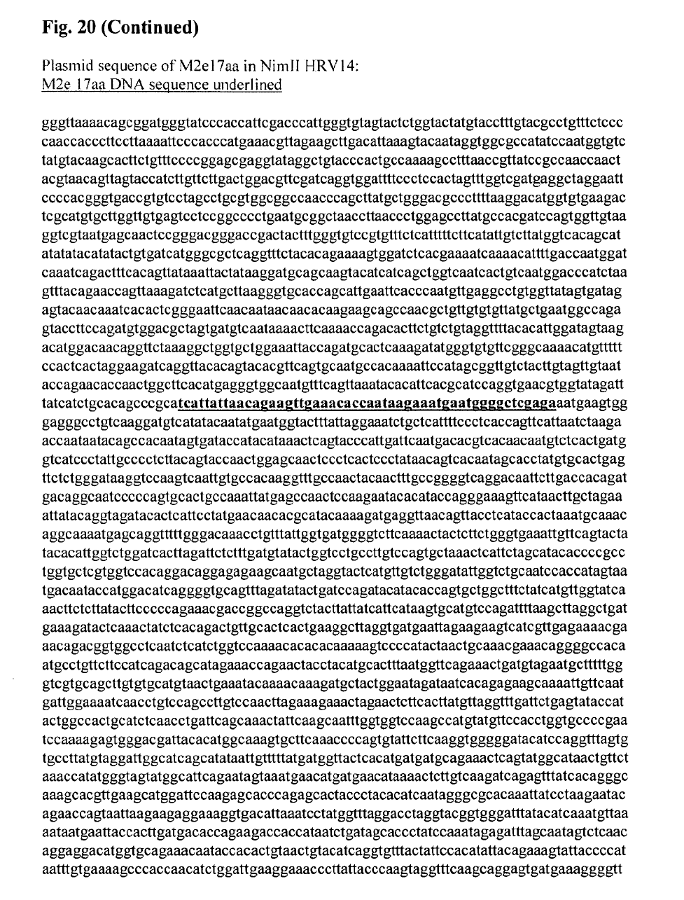

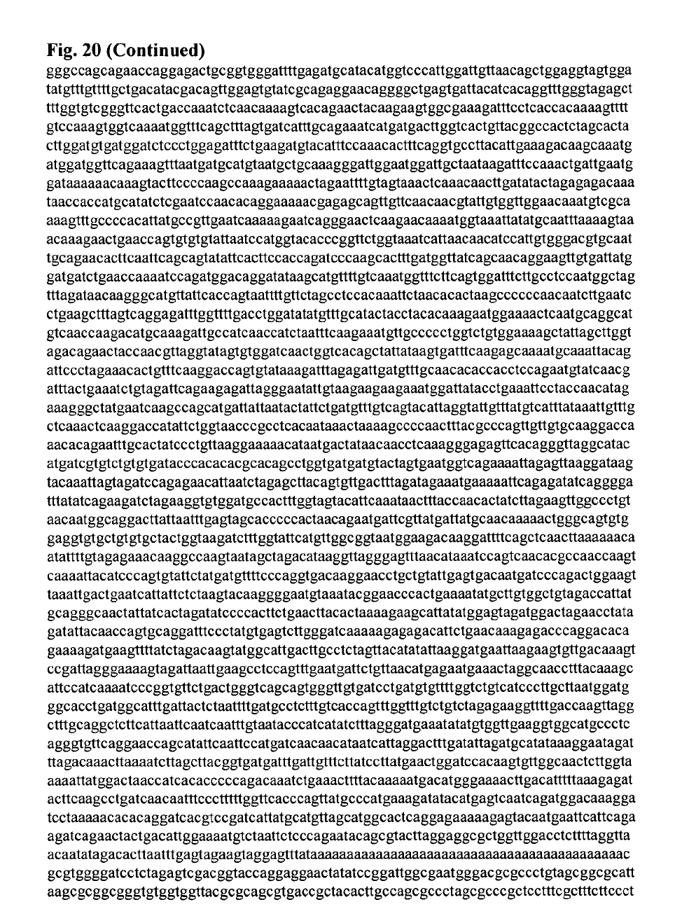

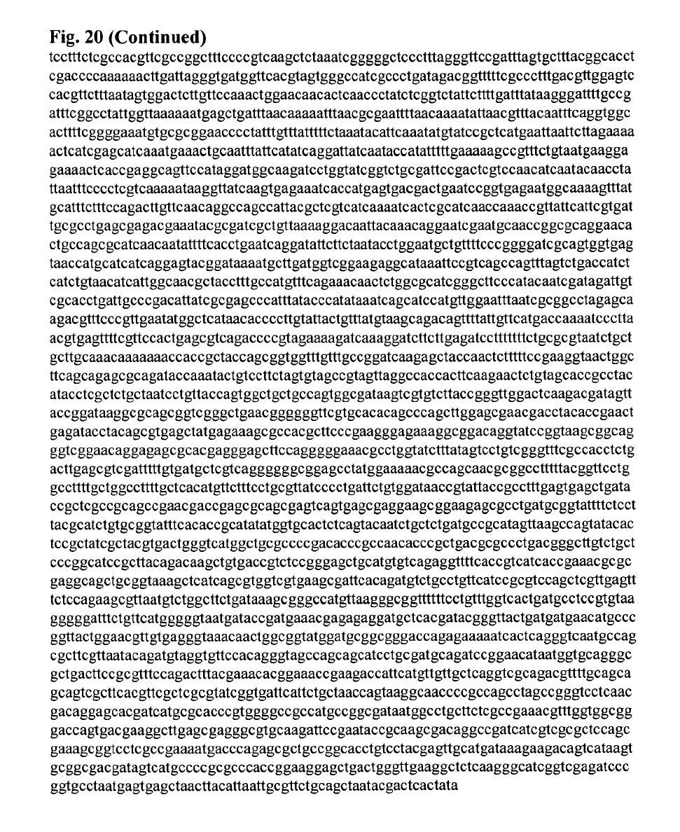

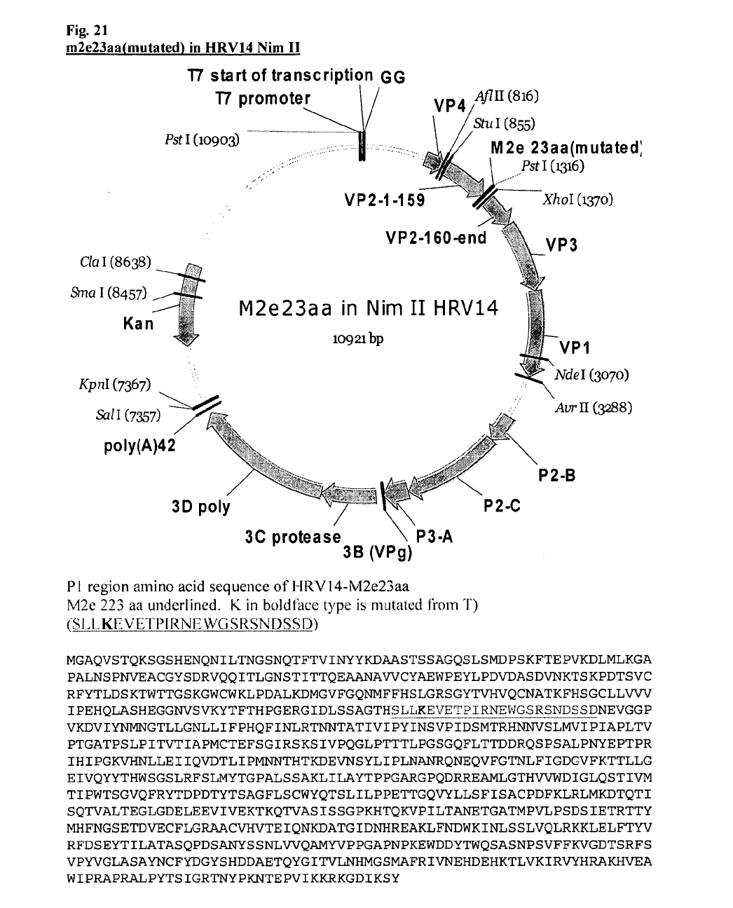

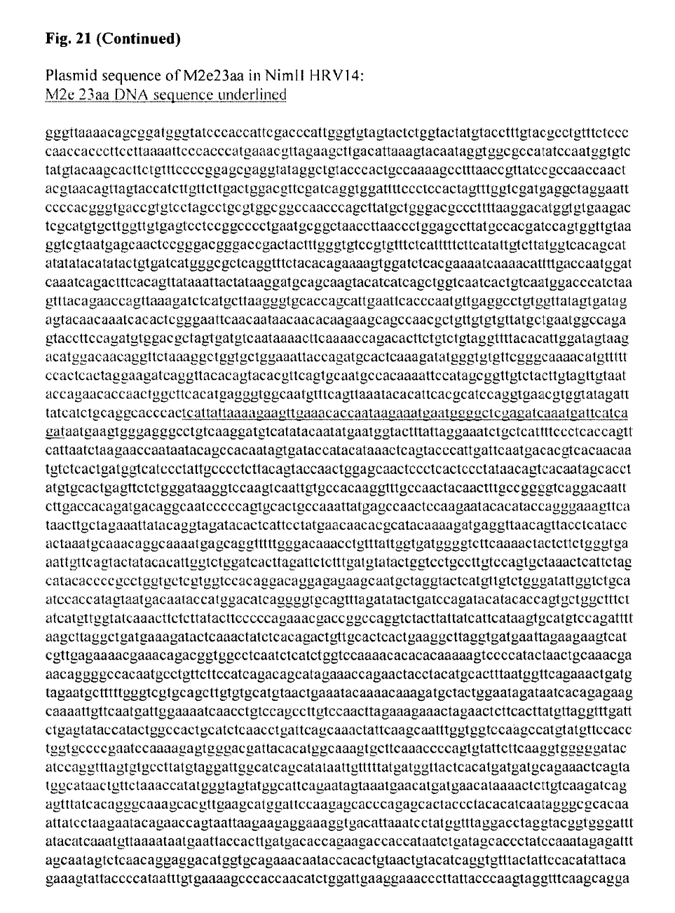

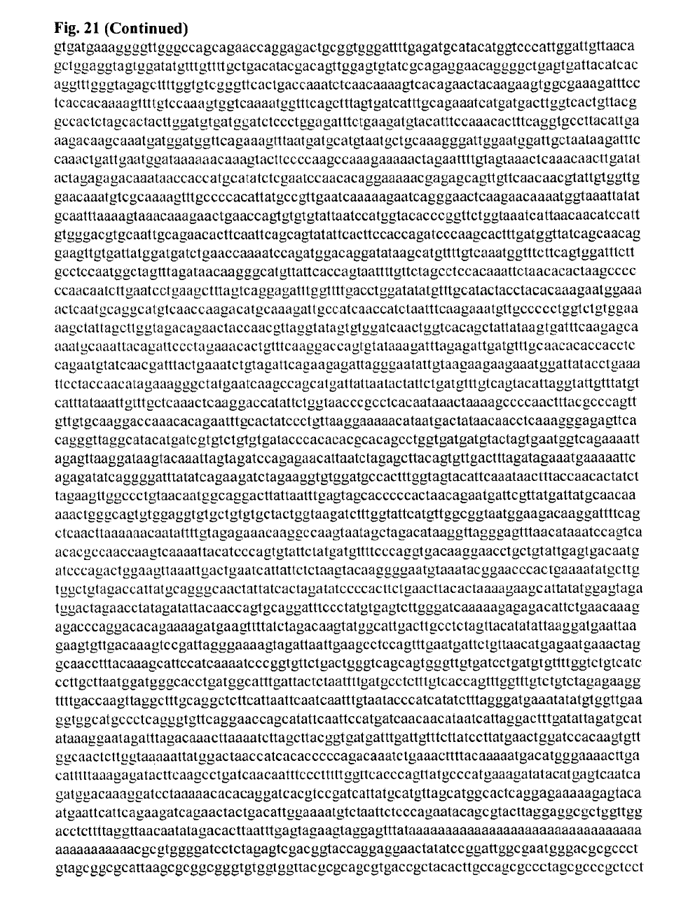

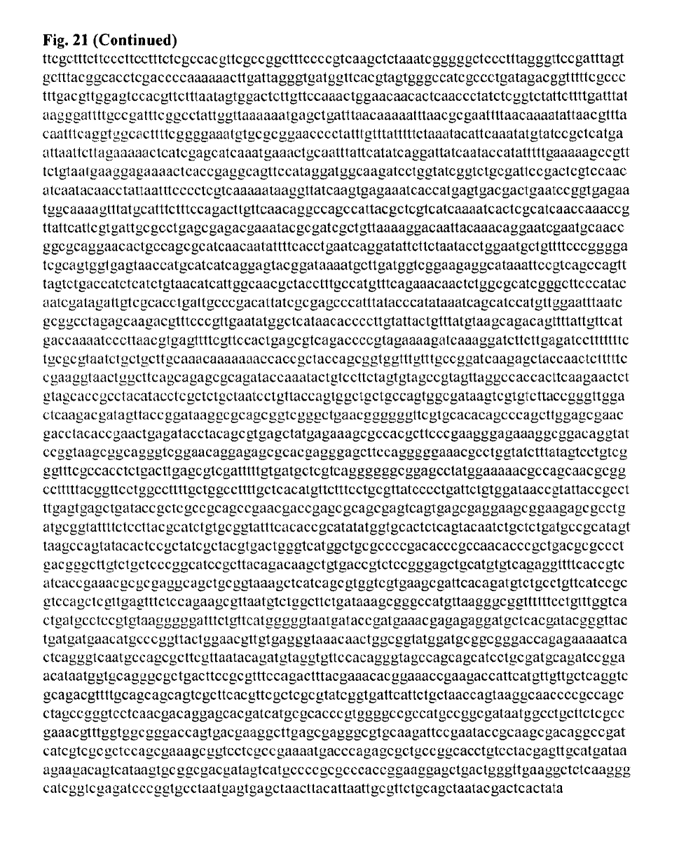

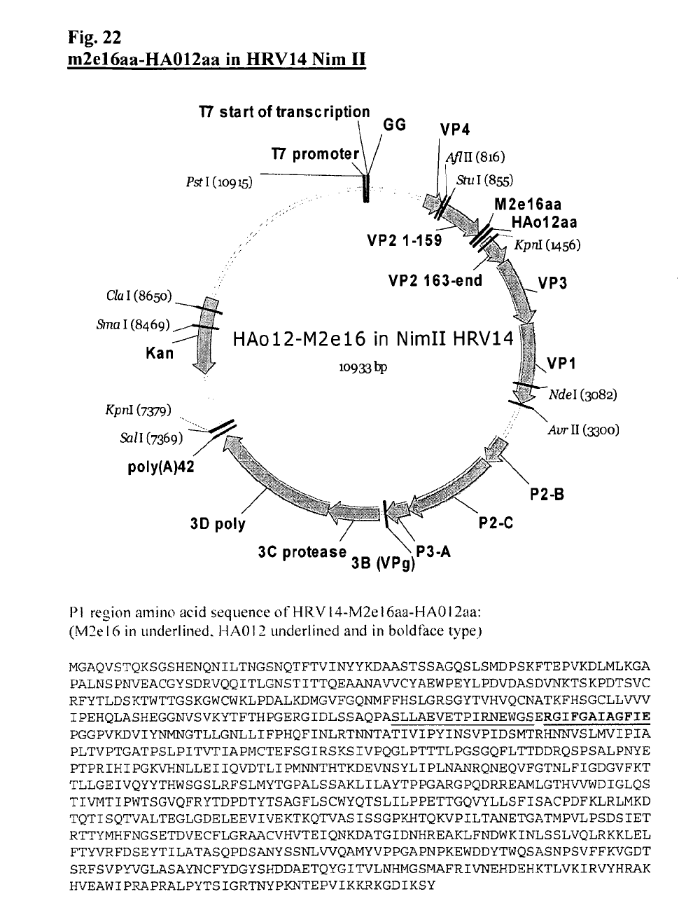

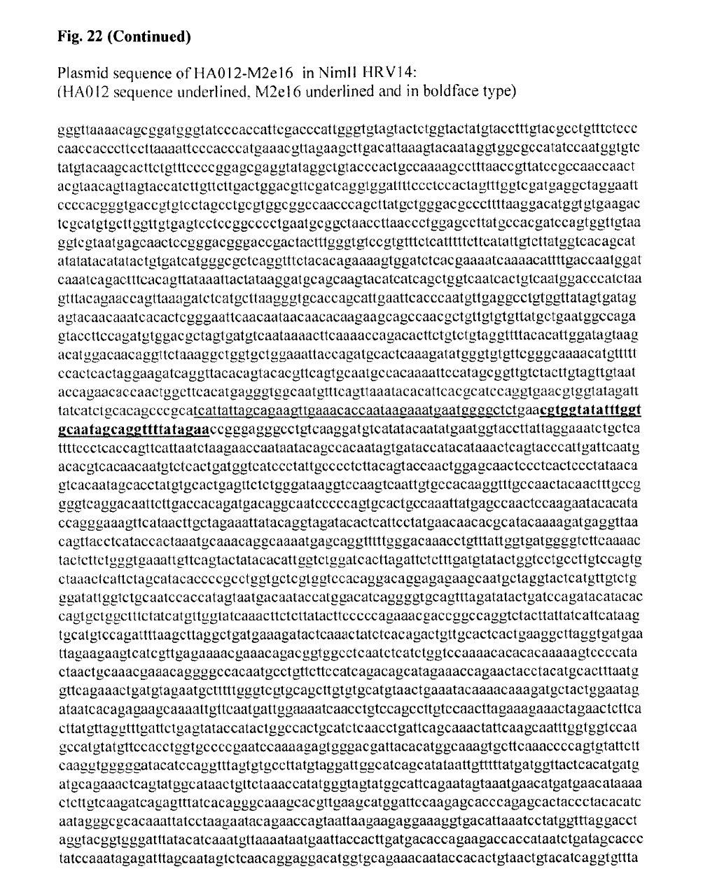

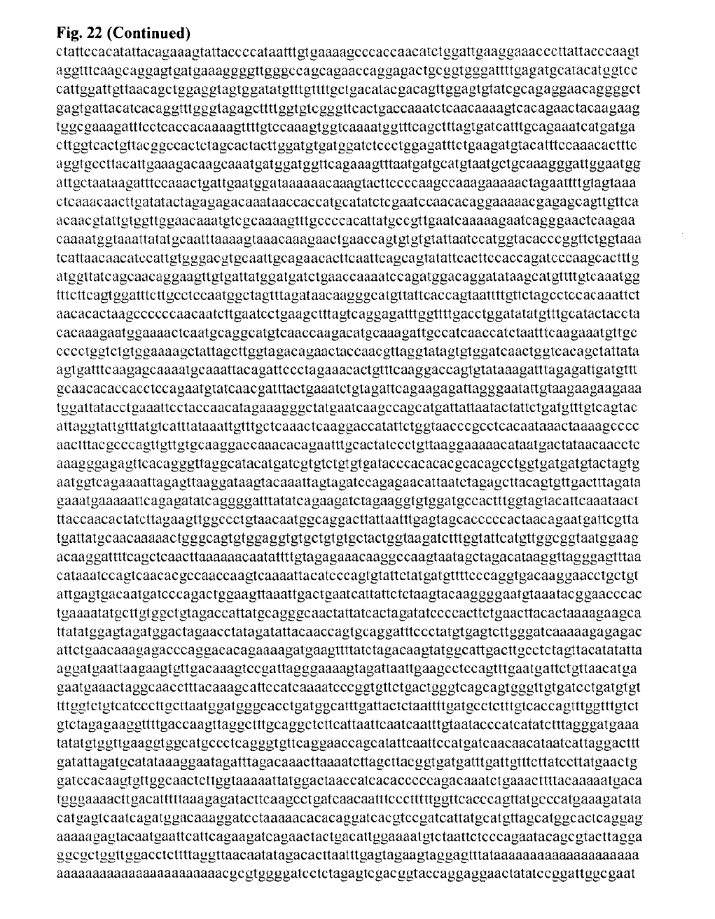

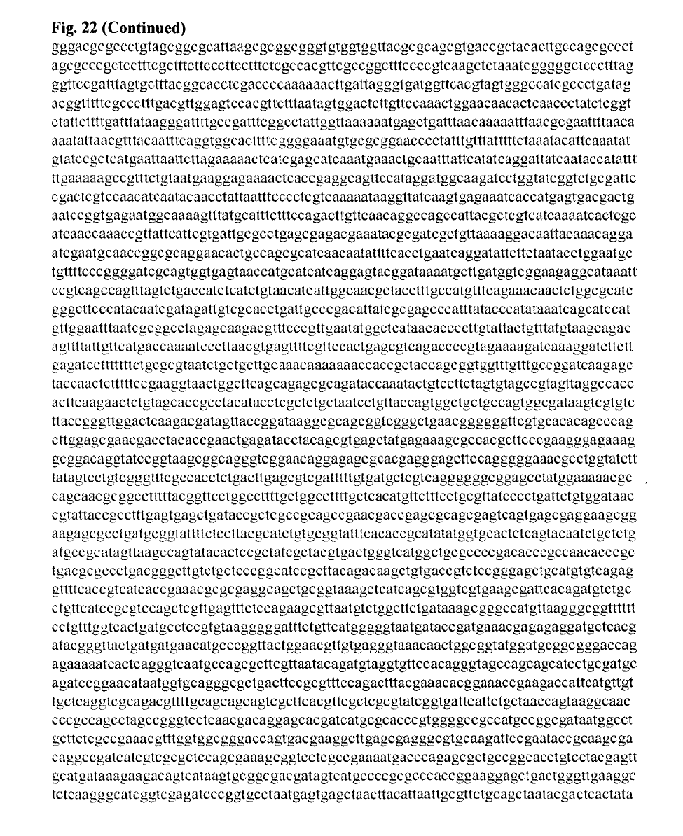

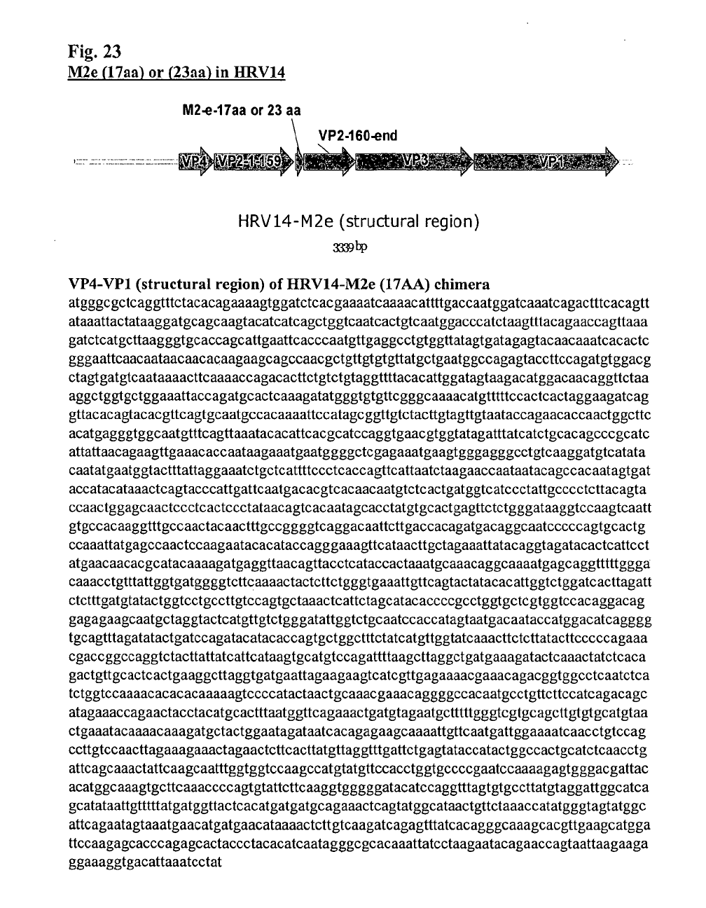

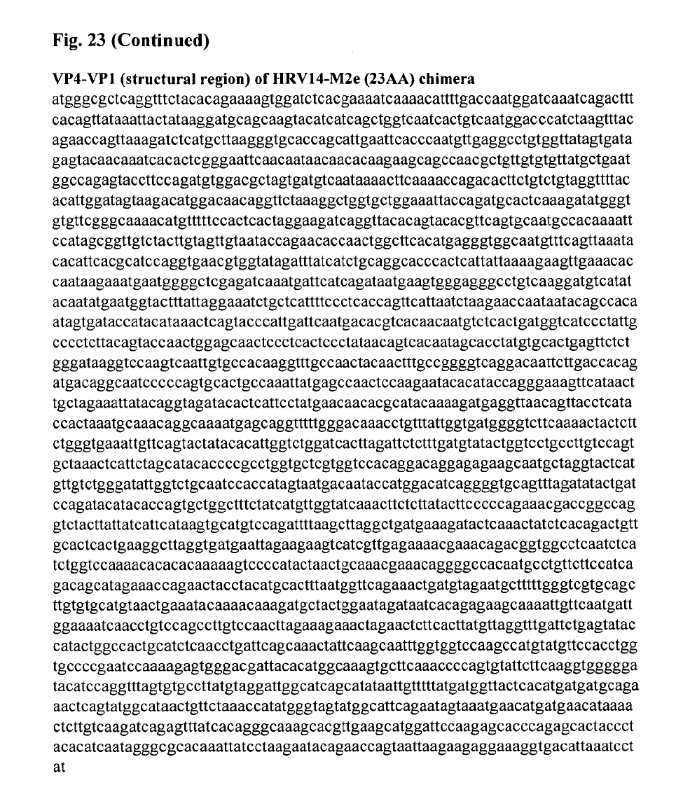

Additional sites at which insertions can be made, alone or in combination with insertions at other sites (e.g., the NimII site), include NimI (A and B), NimIII, and NimIV. Thus, insertions can be made, for example, at positions 91 and/or 95 of VP1 (NimIA), positions 83, 85, 138, and/or 139 of VP1 (NimIB), and/or position 287 of VP1 (NimIII) (see, e.g., FIG. 17). NimIV is in the carboxyl-terminal region of VP1, in a region comprising the following sequence, which represents amino acids 274-289 of HRV14 VP1: NTEPVIKKRKGDIKSY (SEQ ID NO:28). Insertions can be made into this NimIV site or corresponding regions of other HRV serotypes. Insertions between any amino acids in this region are included in the invention. Thus, the invention includes, for example, insertions between amino acids 274 and 275; 275 and 276; 276 and 277; 277 and 278; 278 and 279; 279 and 280; 280 and 281; 281 and 282; 282 and 283; 283 and 284; 284 and 285; 285 and 286; 286 and 287; 287 and 288; and 288 and 289. In addition to these insertions, the invention includes insertions where one or more (e.g., 2, 3, 4, 5, 6, 7, 8, 9, or 10) amino acids in this region are deleted. Thus, for example, the invention includes insertions between amino acids 274 and 276; 275 and 277; 276 and 278; 277 and 279; 278 and 280; 279 and 281; 280 and 282; 281 and 283; 282 and 284; 283 and 285; 284 and 286; 285 and 287; 286 and 288; 287 and 289; 288 and 290; and 289 and 291. The insertions can further be made in place of deletions of, e.g., one, two, three, four, or five amino acids on either or both sides of the indicated amino acids.

The vectors of the invention are made using standard methods of molecular biology, which are exemplified below in the case of a vector including insertions in NimII of HRV14. In addition, and as is discussed further below, the vectors of the invention can be administered in the form of live viruses or can be inactivated prior to administration by, for example, formalin inactivation or ultraviolet treatment, using methods known to those skilled in the art.

Optionally, the vectors can include linker sequences between the HRV vector sequences and the inserted influenza sequences, on the amino and/or carboxyl-terminal ends. These linker sequences can be used to provide flexibility to inserted sequences, enabling the inserted sequences to present the inserted epitope in a manner in which it can induce an immune response. Examples of such linker sequences are provided below. Identification of linker sequences to be used with a particular insert can be carried out by, for example, the library screening method of the invention as described herein. Briefly, in this method, libraries are constructed that have random sequences of various length in a region desired for identification of effective linker sequences. Viruses generated from the library are tested for viability and immunogenicity of the inserted sequences, to identify effective linkers.

The viruses of the invention can be grown using standard methods such as, for example, by passaging in cell cultures. For example, virus can be grown in, and purified from, cells such MRC-5 cells or HeLa cells.

### Heterologous Peptides

The viral vectors of the invention can be used to deliver any peptide, protein, or other amino acid-based immunogen of prophylactic or therapeutic interest. For example, the vectors of the invention can be used in the induction of an immune response (prophylactic or therapeutic) to any protein-based antigen that is inserted into an HRV protein. Prophylaxis and prevention as used herein include administration of immunogenic compositions of the invention to subjects that are not infected with a pathogen from which a peptide or protein inserted into a vector of the invention is derived. Administration of a composition of the invention to such subjects can prevent or substantially prevent the development of symptomatic infection, if such subjects are, after the administration, infected with the pathogen. Thus, the administration can enable the immune system of the subject to prevent or substantially prevent progression of the infection to, for example, a symptomatic stage. Therapeutic administration includes administration to subjects that already are infected with a pathogen from which an inserted peptide or protein is derived. Such subjects may exhibit symptoms of the infection. These terms are equally applicable in the context of tumor-associated antigens. For example, prophylactic or preventative administration can be carried out in patients not having a tumor (or not diagnosed as having a tumor), and such administration can induce an immune response to fight any tumors that develop in the subject. Therapeutic treatment involving administration of a tumor-associated antigen can be carried out in patients already diagnosed with a tumor.

The vectors of the invention can each include a single epitope of an inserted sequence. Alternatively, multiple epitopes can be inserted into the vectors, either at a single site (e.g., as a polytope, in which the different epitopes can optionally be separated by a flexible linker, such as a polyglycine stretch of amino acids or one amino acid as described in the example below), at different sites (e.g., the different Nim sites), or in any combination thereof. The different epitopes can be derived from a single species, strain, or serotype of pathogen (or other source), or can be derived from different species, strains, serotypes, and/or genuses. The vectors can include multiple peptides, for example, multiple copies of peptides as listed herein or combinations of peptides such as those listed herein. As an example, the vectors can include HA₀ and M2e sequences, or human and avian HA₀ and/or M2e peptides (and/or consensus sequences thereof; and/or other peptides such as those described herein).

Immunogens that can be used in the invention can be derived from, for example, infectious agents such as viruses, bacteria, and parasites. A specific example of such an infectious agent is influenza viruses, including those that infect humans (e.g., A, B, and C strains), as well as avian influenza viruses. Examples of immunogens from influenza viruses include those derived from hemagglutinin (HA; e.g., any one of H1-H16, or subunits thereof) (HA₀ or HA subunits HA1 and HA2), M2 (e.g., M2e), neuraminidase (NA; e.g., any one of N1-N9), M1, nucleoprotein (NP), and B proteins.

Examples of sequences that can be included in the vectors of the invention are influenza virus peptides including the hemagglutinin precursor protein cleavage site (HA₀) (NIPSIQSRGLFGAIAGFIE (SEQ ID NO:31) for A/H1 strains, NVPEKQTRGIFGAIAGFIE (SEQ ID NO:32) for A/H3 strains, and PAKLLKERGFFGAIAGFLE (SEQ ID NO:33) for influenza B strains). Two specific examples of such peptides include RGIFGAIAGFI (SEQ ID NO:34) and NVPEKQTQGIFGAIAGFI (SEQ ID NO:35).

In other examples, the HA_{D}-based vaccine includes additional immunogen sequences (e.g., influenza virus M2e sequences) or is administered with additional immunogens (e.g., influenza virus M2e). Examples of such sequences are provided throughout this specification and in Tables 6-9. Specific examples of such sequences include the following: MSLLTEVETPIRNEWGCRCNDSSD (SEQ ID NO:9); MSLLTEVETPTRNEWECRCSDSSD (SEQ ID NO:10); MSLLTEVETLTRNGWGCRCSDSSD (SEQ ID NO:11); EVETPTRN (SEQ ID NO:13); SLLTEVETPIRNEWGCRCNDSSD (SEQ ID NO:36); and SLLTEVETPIRNEWGCR (SEQ ID NO:37). Additional M2e sequences that can be used in invention include sequences from the extracellular domain of BM2 protein of influenza B (consensus MLEPFQ; SEQ ID NO:38), and the M2e peptide from the H5N1 avian flu (MSLLTEVETLTRNGWGCRCSDSSD; SEQ ID NO:11). The following is an example of a combined HA₀ and M2e sequences that can be used in the invention: SLLTEVETPIRNEWGSERGIFGAIAGFIE (SEQ ID NO:39).

In the case of vaccines including more than one immunogen, the multiple immunogens can be included within the same or different delivery vehicles, such as the HRV-based vectors of the invention. The vectors of the invention can be administered in combination with other types of vectors, such as Hepatitis B core-based vectors, as described further herein (also see, e.g., U.S. Pat. No. 7,361,352) and/or subunit vaccines.

Other examples of peptides that are conserved in influenza can be used in the invention in combination with HA₀-based vaccines and include the NBe peptide conserved for influenza B (consensus sequence MNNATFNYTNVNPISHIRGS; SEQ ID NO:40). Further examples of influenza peptides that can be used in the invention, as well as proteins from which such peptides can be derived (e.g., by fragmentation and/or creation of analogs; see below) are described in US 2002/0165176, US 2003/0175290, US 2004/0055024, US 2004/0116664, US 2004/0219170, US 2004/0223976, US 2005/0042229, US 2005/0003349, US 2005/0009008, US 2005/0186621, U.S. Pat. No. 4,752,473, U.S. Pat. No. 5,374,717, U.S. Pat. No. 6,169,175, U.S. Pat. No. 6,720,409, U.S. Pat. No. 6,750,325, U.S. Pat. No. 6,872,395, WO 93/15763, WO 94/06468, WO 94/17826, WO 96/10631, WO 99/07839, WO 99/58658, WO 02/14478, WO 2003/102165, WO 2004/053091, WO 2005/055957, and the Tables 6-9 (and references cited therein), the contents of which are incorporated herein by reference. Further, conserved immunologic/protective T and B cell epitopes of influenza can be chosen from publicly available databases (see, e.g., Bui et al., Proc. Natl. Acad. Sci. U.S.A. 104:246-251, 2007 and supplemental tables). The invention can also employ any peptide from the on-line IEDB resource, e.g., influenza virus epitopes including conserved B and T cell epitopes described in Bui et al., supra.

Protective epitopes from other human/veterinary pathogens, such as epitopes from parasites (e.g., malaria), other pathogenic viruses (e.g., human papilloma virus (HPV), herpes simplex viruses (HSV), human immunodeficiency viruses (HIV; e.g., gag), and hepatitis C viruses (HCV)), and bacteria (e.g., Mycobacterium tuberculosis, Clostridium difficile, and Helicobacter pylori) can also be combined with the HA₀-based vaccines of the invention, or administered in the absence of HA_{O}-based peptides using the vectors of the invention. Various appropriate epitopes of these and other pathogens are known in the art. For example, cross-protective epitopes/peptides from papillomavirus L2 protein inducing broadly cross-neutralizing antibodies that protect from different HPV genotypes can be used, such as peptides including amino acids 1-88, amino acids 1-200, or amino acids 17-36 of L2 protein of, e.g., HPV16 virus (WO 2006/083984 A1; QLYKTCKQAGTCPPDIIPKV; SEQ ID NO:41). Examples of additional pathogens, as well as immunogens and epitopes from these pathogens, which can be used in the invention are provided in WO 2004/053091, WO 03/102165, WO 02/14478, and US 2003/0185854, the contents of which are incorporated herein by reference.

Additional examples of pathogens from which immunogens can be obtained are listed in Table 1, below, and specific examples of such immunogens include those listed in Table 2. In addition, specific examples of epitopes that can be inserted into the vectors of the invention are provided in Table 3. As is noted in Table 3, epitopes that are used in the vectors of the invention can be B cell epitopes (i.e., neutralizing epitopes) or T cell epitopes (i.e., T helper and cytotoxic T cell-specific epitopes).

The vectors of the invention can be used to deliver immunogens in addition to pathogen-derived antigens. For example, the vectors can be used to deliver tumor-associated antigens for use in immunotherapeutic methods against cancer. Numerous tumor-associated antigens are known in the art and can be administered according to the invention. Examples of cancers (and corresponding tumor associated antigens) are as follows: melanoma (NY-ESO-1 protein (specifically CTL epitope located at amino acid positions 157-165), CAMEL, MART 1, gp100, tyrosine-related proteins TRP1 and 2, and MUC1); adenocarcinoma (ErbB2 protein); colorectal cancer (17-1A, 791Tgp72, and carcinoembryonic antigen); prostate cancer (PSA1 and PSA3). Heat shock protein (hsp110) can also be used as such an immunogen.

In another example of the invention, exogenous sequences that encode an epitope(s) of an allergy-inducing antigen to which an immune response is desired can be used. In addition, the vectors of the invention can include ligands that are used to target the vectors to deliver peptides, such as antigens, to particular cells (e.g., cells that include receptors for the ligands) in subjects to whom the vectors administered.

Further examples of pathogen, tumor, and allergen-related peptides and sources thereof that can be included as immunogens in the vectors of the invention are described as follows. These peptide immunogens can be used in combination with each other and/or other peptides described herein (e.g., HA₀ and/or M2e-related sequences, such as those described herein). The invention includes compositions including these vectors, as well as methods of using the vectors to induce immune responses against the immunogens. Thus, for example, in addition to the immunogens described above, the vectors described herein can include one or more immunogen(s) derived from or that direct an immune response against one or more viruses (e.g., viral target antigen(s)) including, for example, a dsDNA virus (e.g., adenovirus, herpesvirus, epstein-barr virus, herpes simplex type 1, herpes simplex type 2, human herpes virus simplex type 8, human cytomegalovirus, varicella-zoster virus, poxvirus); ssDNA virus (e.g., parvovirus, papillomavirus (e.g., E1, E2, E3, E4, E5, E6, E7, E8, BPV1, BPV2, BPV3, BPV4, BPV5, and BPV6 (In Papillomavirus and Human Cancer, edited by H. Pfister (CRC Press, Inc. 1990)); Lancaster et al., Cancer Metast. Rev. pp. 6653-6664, 1987; Pfister et al., Adv. Cancer Res. 48:113-147, 1987)); dsRNA viruses (e.g., reovirus); (+)ssRNA viruses (e.g., picornavirus, coxsackie virus, hepatitis A virus, poliovirus, togavirus, rubella virus, flavivirus, hepatitis C virus, yellow fever virus, dengue virus, west Nile virus); (−)ssRNA viruses (e.g., orthomyxovirus, influenza virus, rhabdovirus, paramyxovirus, measles virus, mumps virus, parainfluenza virus, rhabdovirus, rabies virus); ssRNA-RT viruses (e.g., retrovirus, human immunodeficiency virus (HIV)); and dsDNA-RT viruses (e.g. hepadnavirus, hepatitis B). Immunogens can also be derived from other viruses not listed above but available to those of skill in the art.

With respect to HIV, immunogens can be selected from any HIV isolate. As is well-known in the art, HIV isolates are now classified into discrete genetic subtypes. HIV-1 is known to comprise at least ten subtypes (A, B, C, D, E, F, G, H, J, and K). HIV-2 is known to include at least five subtypes (A, B, C, D, and E). Subtype B has been associated with the HIV epidemic in homosexual men and intravenous drug users worldwide. Most HIV-1 immunogens, laboratory adapted isolates, reagents and mapped epitopes belong to subtype B. In sub-Saharan Africa, India, and China, areas where the incidence of new HIV infections is high, HIV-1 subtype B accounts for only a small minority of infections, and subtype HIV-1 C appears to be the most common infecting subtype. Thus, in certain embodiments, it may be desirable to select immunogens from HIV-1 subtypes B and/or C. It may be desirable to include immunogens from multiple HIV subtypes (e.g., HIV-1 subtypes B and C, HIV-2 subtypes A and B, or a combination of HIV-1 and HIV-2 subtypes) in a single immunological composition. Suitable HIV immunogens include ENV, GAG, POL, NEF, as well as variants, derivatives, and fusion proteins thereof, for example.

Immunogens can also be derived from or direct an immune response against one or more bacterial species (spp.) (e.g., bacterial target antigen(s)) including, for example, Bacillus spp. (e.g., Bacillus anthracis), Bordetella spp. (e.g., Bordetella pertussis), Borrelia spp. (e.g., Borrelia burgdorferi), Brucella spp. (e.g., Brucella abortus, Brucella canis, Brucella melitensis, Brucella suis), Campylobacter spp. (e.g., Campylobacter jejuni), Chlamydia spp. (e.g., Chlamydia pneumoniae, Chlamydia psittaci, Chlamydia trachomatis), Clostridium spp. (e.g., Clostridium botulinum, Clostridium difficile, Clostridium perfringens, Clostridium tetani), Corynebacterium spp. (e.g., Corynebacterium diptheriae), Enterococcus spp. (e.g., Enterococcus faecalis, enterococcus faecum), Escherichia spp. (e.g., Escherichia coli), Francisella spp. (e.g., Francisella tularensis), Haemophilus spp. (e.g., Haemophilus influenza), Helicobacter spp. (e.g., Helicobacter pylori), Legionella spp. (e.g., Legionella pneumophila), Leptospira spp. (e.g., Leptospira interrogans), Listeria spp. (e.g., Listeria monocytogenes), Mycobacterium spp. (e.g., Mycobacterium leprae, Mycobacterium tuberculosis), Mycoplasma spp. (e.g., Mycoplasma pneumoniae), Neisseria spp. (e.g., Neisseria gonorrhea, Neisseria meningitidis), Pseudomonas spp. (e.g., Pseudomonas aeruginosa), Rickettsia spp. (e.g., Rickettsia rickettsii), Salmonella spp. (e.g., Salmonella typhi, Salmonella typhinurium), Shigella spp. (e.g., Shigella sonnei), Staphylococcus spp. (e.g., Staphylococcus aureus, Staphylococcus epidermidis, Staphylococcus saprophyticus, coagulase negative staphylococcus (e.g., U.S. Pat. No. 7,473,762)), Streptococcus spp. (e.g., Streptococcus agalactiae, Streptococcus pneumoniae, Streptococcus pyrogenes), Treponema spp. (e.g., Treponema pallidum), Vibrio spp. (e.g., Vibrio cholerae), and Yersinia spp. (Yersinia pestis). Immunogens can also be derived from or direct the immune response against other bacterial species not listed above but available to those of skill in the art.

Immunogens can also be derived from or direct an immune response against one or more parasitic organisms (spp.) (e.g., parasite target antigen(s)) including, for example, Ancylostoma spp. (e.g., A. duodenale), Anisakis spp., Ascaris lumbricoides, Balantidium coli, Cestoda spp., Cimicidae spp., Clonorchis sinensis, Dicrocoelium dendriticum, Dicrocoelium hospes, Diphyllobothrium latum, Dracunculus spp., Echinococcus spp. (e.g., E. granulosus, E. multilocularis), Entamoeba histolytica, Enterobius vermicularis, Fasciola spp. (e.g., F. hepatica, F. magna, F. gigantica, F. jacksoni), Fasciolopsis buski, Giardia spp. (Giardia lamblia), Gnathostoma spp., Hymenolepis spp. (e.g., H. nana, H. diminuta), Leishmania spp., Loa loa, Metorchis spp. (M. conjunctus, M. albidus), Necator americanus, Oestroidea spp. (e.g., botfly), Onchocercidae spp., Opisthorchis spp. (e.g., O. viverrini, O. felineus, O. guayaquilensis, and O. noverca), Plasmodium spp. (e.g., P. falciparum), Protofasciola robusta, Parafasciolopsis fasciomorphae, Paragonimus westermani, Schistosoma spp. (e.g., S. mansoni, S. japonicum, S. mekongi, S. haematobium), Spirometra erinaceieuropaei, Strongyloides stercoralis, Taenia spp. (e.g., T. saginata, T. solium), Toxocara spp. (e.g., T. canis, T. cati), Toxoplasma spp. (e.g., T. gondii), Trichobilharzia regenti, Trichinella spiralis, Trichuris trichiura, Trombiculidae spp., Trypanosoma spp., Tunga penetrans, and/or Wuchereria bancrofti. Immunogens can also be derived from or direct the immune response against other parasitic organisms not listed above but available to those of skill in the art.

Immunogens can be derived from or direct the immune response against tumor target antigens (e.g., tumor target antigens). The term tumor target antigen (TA) can include both tumor-associated antigens (TAAs) and tumor-specific antigens (TSAs), where a cancerous cell is the source of the antigen. A TA can be an antigen that is expressed on the surface of a tumor cell in higher amounts than is observed on normal cells or an antigen that is expressed on normal cells during fetal development. A TSA is typically an antigen that is unique to tumor cells and is not expressed on normal cells. TAs are typically classified into five categories according to their expression pattern, function, or genetic origin: cancer-testis (CT) antigens (i.e., MAGE, NY-ESO-1); melanocyte differentiation antigens (e.g., Melan A/MART-1, tyrosinase, gp100); mutational antigens (e.g., MUM-1, p53, CDK-4); overexpressed ‘self’ antigens (e.g., HER-2/neu, p53); and viral antigens (e.g., HPV, EBV). Suitable TAs include, for example, gp100 (Cox et al., Science 264:716-719, 1994), MART-1/Melan A (Kawakami et al., J. Exp. Med., 180:347-352, 1994), gp75 (TRP-1) (Wang et al., J. Exp. Med., 186:1131-1140, 1996), tyrosinase (Wolfel et al., Eur. J. Immunol., 24:759-764, 1994), NY-ESO-1 (WO 98/14464; WO 99/18206), melanoma proteoglycan (Hellstrom et al., J. Immunol., 130:1467-1472, 1983), MAGE family antigens (e.g., MAGE-1, 2, 3, 4, 6, and 12; Van der Bruggen et al., Science 254:1643-1647, 1991; U.S. Pat. No. 6,235,525), BAGE family antigens (Boel et al., Immunity 2:167-175, 1995), GAGE family antigens (e.g., GAGE-1,2; Van den Eynde et al., J. Exp. Med. 182:689-698, 1995; U.S. Pat. No. 6,013,765), RAGE family antigens (e.g., RAGE-1; Gaugler et al., Immunogenetics 44:323-330, 1996; U.S. Pat. No. 5,939,526), N-acetylglucosaminyltransferase-V (Guilloux et al., J. Exp. Med. 183:1173-1183, 1996), p15 (Robbins et al., J. Immunol. 154:5944-5950, 1995), B-catenin (Robbins et al., J. Exp. Med., 183:1185-1192, 1996), MUM-1 (Coulie et al., Proc. Natl. Acad. Sci. U.S.A. 92:7976-7980, 1995), cyclin dependent kinase-4 (CDK4) (Wolfel et al., Science 269:1281-1284, 1995), p21-ras (Fossum et al., Int. J. Cancer 56:40-45, 1994), BCR-abl (Bocchia et al., Blood 85:2680-2684, 1995), p53 (Theobald et al., Proc. Natl. Acad. Sci. U.S.A. 92:11993-11997, 1995), p185 HER2/neu (erb-B1; Fisk et al., J. Exp. Med., 181:2109-2117, 1995), epidermal growth factor receptor (EGFR) (Harris et al., Breast Cancer Res. Treat, 29:1-2, 1994), carcinoembryonic antigens (CEA) (Kwong et al., J. Natl. Cancer Inst., 85:982-990, 1995) U.S. Pat. Nos. 5,756,103; 5,274,087; 5,571,710; 6,071,716; 5,698,530; 6,045,802; EP 263933; EP 346710; and EP 784483; carcinoma-associated mutated mucins (e.g., MUC-1 gene products; Jerome et al., J. Immunol., 151:1654-1662, 1993); EBNA gene products of EBV (e.g., EBNA-1; Rickinson et al., Cancer Surveys 13:53-80, 1992); E7, E6 proteins of human papillomavirus (Ressing et al., J. Immunol. 154:5934-5943, 1995); prostate specific antigen (PSA; Xue et al., The Prostate 30:73-78, 1997); prostate specific membrane antigen (PSMA; Israeli et al., Cancer Res. 54:1807-1811, 1994); idiotypic epitopes or antigens, for example, immunoglobulin idiotypes or T cell receptor idiotypes (Chen et al., J. Immunol. 153:4775-4787, 1994); KSA (U.S. Pat. No. 5,348,887), kinesin 2 (Dietz, et al., Biochem. Biophys. Res. Commun. 275(3):731-738, 2000), HIP-55, TG93-1 anti-apoptotic factor (Toomey et al., Br. J. Biomed. Sci. 58(3):177-183, 2001), tumor protein D52 (Bryne et al., Genomics 35:523-532, 1996), H1FT, NY-BR-1 (WO 01/47959), NY-BR-62, NY-BR-75, NY-BR-85, NY-BR-87, and NY-BR-96 (Scanlan, M. Serologic and Bioinformatic Approaches to the Identification of Human Tumor Antigens, in Cancer Vaccines 2000, Cancer Research Institute, New York, N.Y.), and/or pancreatic cancer antigens (e.g., SEQ ID NOs: 1-288 of U.S. Pat. No. 7,473,531). Immunogens can also be derived from or direct the immune response against include TAs not listed above but available to one of skill in the art.

The size of the peptide or protein that is inserted into the vectors of the invention can range in length from, for example, from 3-1,000 amino acids, for example, from 5-500, 10-100, 20-55, 25-45, or 35-40 amino acids, as can be determined to be appropriate by those of skill in the art. Thus, for example, peptides in the range of 7-45, 10-40, 12-30, and 15-25 amino acids in length can be used in the invention. The peptides included in the vectors of the invention can include complete sequences, as specified and referenced herein, or fragments including one or more epitopes capable of inducing the desired immune response. Such fragments can include, e.g., 2-50, 3-40, 4-30, 5-25, or 6-20 amino acid fragments from within these peptides. Further, the peptides can include truncations or extensions of the sequences (e.g., insertion of additional/repeat immunodominant/helper epitopes) by, e.g., 1, 2, 3, 4, 5, 6, 7, 8, 9, 10, 11-20, etc., amino acids on either or both ends, including, for example, naturally occurring, contiguous sequences (e.g., the sequences with which the peptides are contiguous in the influenza virus (or other source) genome), or synthetic linker sequences (also see below). The peptides can thus include, e.g., 1-25, 2-20, 3-15, 4-10, or 4-8 amino acid sequences on one or both ends. As specific examples, the peptides can include 1-3 amino acid linker sequences at amino and/or carboxyl terminal ends. Truncations of the peptides or proteins can remove immunologically unimportant or interfering sequences, e.g., within known structural/immunologic domains, or between domains; or whole undesired domains can be deleted; such modifications can be in the ranges 21-30, 31-50, 51-100, 101-400, etc. amino acids. The ranges also include, e.g., 20-400, 30-100, and 50-100 amino acids. Further, the sequences can include deletions or substitutions of, e.g., 1, 2, 3, 4, 5, 6, 7, 8, 9, 10, or more amino acids (e.g., 1-50, 3-40, 5-30, 8-25, 10-20, or 12-15 amino acids) from within and/or at either or both ends of the peptide. All such possible peptide fragments of the sequences noted above are included in the invention. Thus, in addition to the specific peptides sequences listed and referenced herein (and truncations and extensions thereof), the invention also includes analogs of the sequences. Such analogs include sequences that are, for example, at least 80%, 90%, 95%, or 99% identical to the reference sequences, or fragments thereof. Determination of percentage identity can be carried out using standard methods and software such as, for example, Sequence Analysis Software Package of the Genetics Computer Group, University of Wisconsin Biotechnology Center, 1710 University Avenue, Madison, Wis. 53705, BLAST, or PILEUP/PRETTYBOX programs). These software programs match identical or similar sequences by assigning degrees of identity to various substitutions, deletions, or other modifications. The analogs can include conservative amino acid substitutions in various examples. Conservative substitutions typically include substitutions within the following groups: glycine, alanine, valine, isoleucine, and leucine; aspartic acid, glutamic acid, asparagine, and glutamine; serine and threonine; lysine and arginine; and phenylalanine and tyrosine.

The fragments and analogs described herein can be tested for immunogenicity in standard immunological assays and animal model systems, such as those described herein.

### Administration

When used in immunization methods, the vectors of the invention can be administered as primary prophylactic agents in adults or children at risk of infection by a particular pathogen, such as for example influenza virus. The vectors can also be used as secondary agents for treating infected subjects by stimulating an immune response against the pathogen (or other source) from which the peptide antigen is derived. In the context of immunization against cancer, the vaccines can be administered against subjects at risk of developing cancer or to subjects that already have cancer. In addition to human subjects, the methods of the invention can also involve administration to non-human animals (e.g., livestock, such as, cattle, pigs, horses, sheep, goats, and birds (e.g., chickens, turkeys, ducks, or geese), and domestic animals, including dogs, cats, and birds).

For immunization applications, optionally, adjuvants that are known to those skilled in the art can be used. Adjuvants are selected based on the route of administration. In the case of intranasal administration, chitin microparticles (CMP) can be used (Asahi-Ozaki et al., Microbes and Infection 8:2706-2714, 2006; Ozdemir et al., Clinical and Experimental Allergy 36:960-968, 2006; Strong et al., Clinical and Experimental Allergy 32:1794-1800, 2002). Other adjuvants suitable for use in administration via the mucosal route (e.g., intranasal or oral routes) include the heat-labile toxin of E. coli (LT) or mutant derivatives thereof. In the case of inactivated virus, parenteral adjuvants can be used including, for example, aluminum compounds (e.g., an aluminum hydroxide, aluminum phosphate, or aluminum hydroxyphosphate compound), liposomal formulations, synthetic adjuvants, such as QS21, muramyl dipeptide, monophosphoryl lipid A, or polyphosphazine.

In addition, genes encoding cytokines that have adjuvant activities can be inserted into the vectors. Thus, genes encoding cytokines, such as GM-CSF, IL-2, IL-12, IL-13, or IL-5, can be inserted together with foreign antigen genes to produce a vaccine that results in enhanced immune responses, or to modulate immunity directed more specifically towards cellular, humoral, or mucosal responses. Alternatively, cytokines can be delivered, simultaneously or sequentially, separately from a recombinant vaccine virus by means that are well known (e.g., direct inoculation, naked DNA, in a viral vector, etc.).

The viruses of the invention can be used in combination with other immunization approaches. For example, the viruses can be administered in combination with subunit vaccines including the same or different antigens. The combination methods of the invention can include co-administration of viruses of the invention with other forms of the antigen (or other antigens). For example, subunit forms or delivery vehicles including hepatitis core protein or inactivated whole or partial virus can be used. In one such example, hepatitis B core particles containing M2e peptide on the surface produced in E. coli can be used (HBc-M2e; Fiers et al., Virus Res. 103:173-176, 2004; WO 2005/055957; US 2003/0138769 A1; US 2004/0146524A1; US 2007/0036826 A1).

In another such example, hepatitis B core particles containing HA_{O} peptides are used. Hepatitis B core sequences that can be used to make such particles include full-length sequences, as well as truncated sequences (e.g., carboxy-terminal truncated sequences, truncated at, e.g., amino acid 149, 150, 163, or 164; see, e.g., U.S. Pat. No. 7,361,352). The influenza virus sequences can be inserted within the HBc sequences or at either end of the HBc sequences. For example, sequences can be inserted into the major immunodominant region (MIR) of HBc, which is at about amino acid positions 75-83 of HBc. The insertions into the MIR region can be between any amino acids in this region (e.g., 75-76, 76-77, 77-78, 78-79, 79-80, 80-81, 81-82, or 82-83), or can be present in the place of deletions (of, e.g., 1, 2, 3, 4, 5, 6, or 7 amino acids) in this region (e.g., insertion of influenza B virus sequences between amino acids 78 and 82 of HBc sequences). In another example, insertions are made at the amino-terminus of the HBc protein.

Alternatively, the vectors of the present invention can be used in combination with other approaches (such as subunit or HBc approaches) in a prime-boost strategy, with either the vectors of the invention or the other approaches being used as the prime, followed by use of the other approach as the boost, or the reverse. Further, the invention includes prime-boost strategies employing the vectors of the present invention as both prime and boost agents. Thus, such methods can involve an initial administration of a vector according to the invention, with one or more (e.g., 1, 2, 3, or 4) follow-up administrations that can take place one or more weeks, months, or years after the initial administration.

The vectors of the invention can be administered to subjects as live, live-attenuated, or killed vaccines using standard methods. The live vaccines can be administered intranasally, for example, using methods known to those of skill in the art (see, e.g., Grunberg et al., Am. J. Respir. Crit. Car. Med. 156:609-616, 1997). In the case of intranasal administration, the vectors can be administered in the form of nose-drops or by inhalation of an aerosolized or nebulized formulation. The viruses can be in lyophilized form or dissolved in a physiologically compatible solution or buffer, such as saline or water. Standard methods of preparation and formulation can be used as described, for example, in Remington's Pharmaceutical Sciences (18^{th} edition), ed. A. Gennaro, 1990, Mack Publishing Company, Easton, Pa. Further, determination of an appropriate dosage amount and regimen can readily be determined by those of skill in the art. Appropriate dosage amounts and regimens can readily be determined by those of skill in the art. As an example, the dose range can be, e.g., 10³ to 10⁸ pfu per dose, but can be as low as one TCID50. The vaccine can advantageously be administered in a single dose, however, as noted above, boosting can be carried out as well, if determined to be necessary by those skilled in the art. As to inactivated vaccines, the virus can be killed with, e.g., formalin or UV treatment, and administered intranasally at about 10⁸ pfu per dose (as determined, for example, prior to inactivation), optionally with an appropriate adjuvant (e.g., chitin or mutant LT; see above). In another example, inactivated vaccines can also be administered by a parenteral route, e.g., by subcutaneous administration, optionally with an appropriate adjuvant (e.g., an aluminum adjuvant, such as aluminum hydroxide). In such approaches, it may be advantageous to administer more than one (e.g., 2-3) dose.

The invention is based, in part, on the following experimental examples.

### Experimental Examples

### I. Construction of HRV14-NimII-M2e Chimeras

We have constructed HRV14 NimII-M2e recombinant viruses. The viruses have been shown to express M2e on the virion surface, as demonstrated by the ability of anti-M2e monoclonal antibodies to neutralize the infectivity of the recombinant viruses.

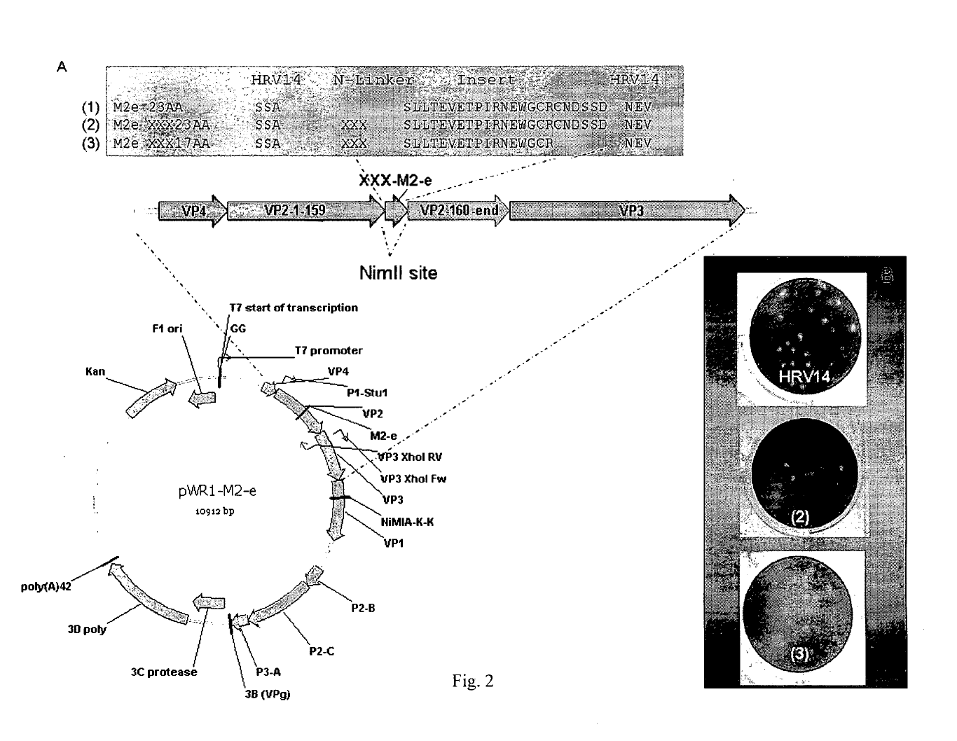

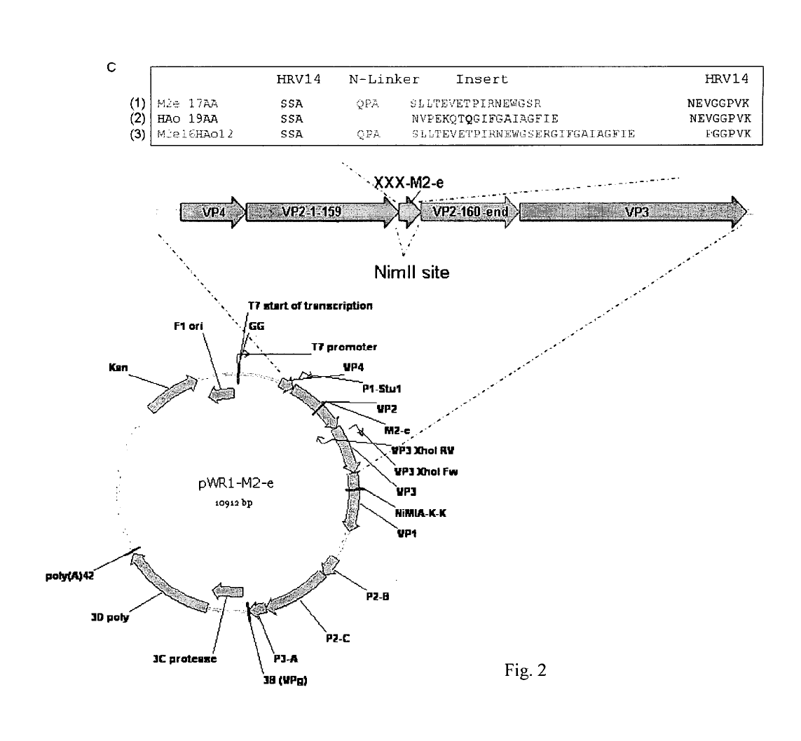

Three types of HRV14-M2e constructs were created (FIG. 2A).

1. HRV14-NimII-23AA, carrying 23 amino acids of M2e inserted between amino acids 159 and 160 of VP2 (NimII site);

2. HRV14-NimII-XXX23AA library. This set of constructs (plasmid library) was similar to the first construct, except for the presence of a 3-amino acid randomized N-terminal linker fused to the peptide. This randomized linker was generated by the M2e sequence using a 5′ (direct) primer containing 9 randomized nucleotides coding for the linker amino acids; and 3. HRV14-NimII-XXX17AA library. This library was generated the same way as the first, but contained a shortened M2e peptide containing only the first 17 amino acids of M2e.

To facilitate cloning into the HRV14 infectious clone, we modified the pWR3.26 infectious clone (Lee et al., J. Virol. 67:2110-2122, 1993) by replacing its pUC plasmid backbone with that of the pEt vector (Novagen) to generate plasmid pWR1 (FIG. 2). Plaque morphology of virus libraries #2 and #3 differed from that of the HRV14 parent (FIG. 2B). The plaque size of the libraries appeared to be similar to wild type, but the plaques were opaque. Construct #1 did not form plaques upon transfection.

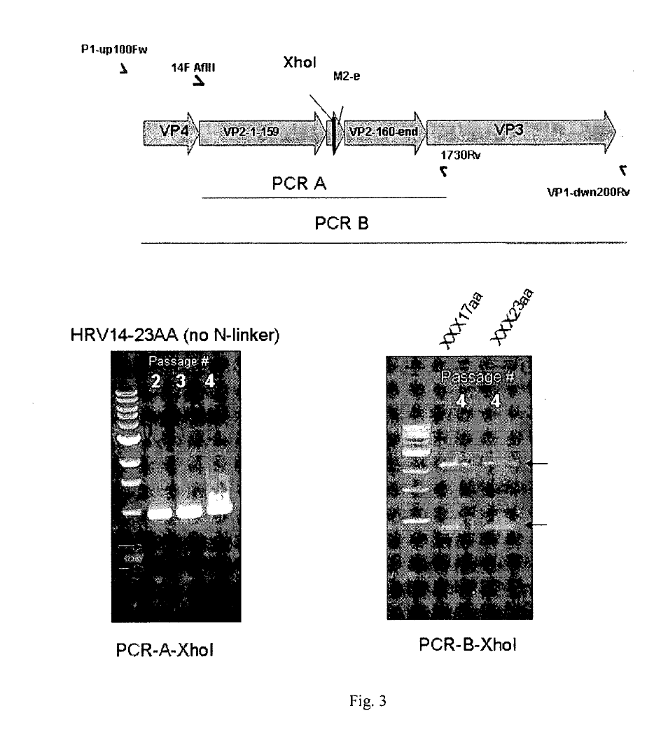

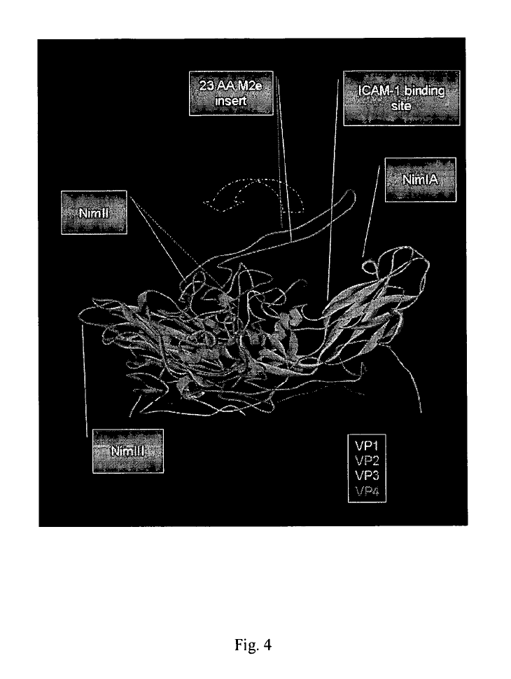

To monitor genetic stability of the constructed viruses, we incorporated an XhoI cleavage site in the middle of the M2e sequence by silent mutagenesis. An RT-PCR fragment obtained from virus containing mutated M2e gene is cleaved by XhoI, while the corresponding DNA product produced from wild type HRV14 remains undigested (FIG. 3). The HRV14-NimII-23AA chimeric construct (#1) resulted in viable, but rather unstable virus. As shown in FIG. 3, the two XhoI digestion products of “PCR A” fragment are detectable only at passage 2, but not at following passages. Libraries (#2) and (#3), on the contrary, stably maintained the M2e insert: fragments “PCR B” obtained from virus libraries at the 4^{th} passage in H1 HeLa cells were completely digested by XhoI (FIG. 3). The instability of construct #1 could be due to steric interference of the inserted peptide with the receptor binding domain (FIG. 4), which may be alleviated when a degenerate linker is provided, as in constructs #2 and #3. The randomized N-terminal linker may have redirected the peptide away from the canyon containing the receptor binding domain allowing efficient virus binding to its receptor (FIG. 4).

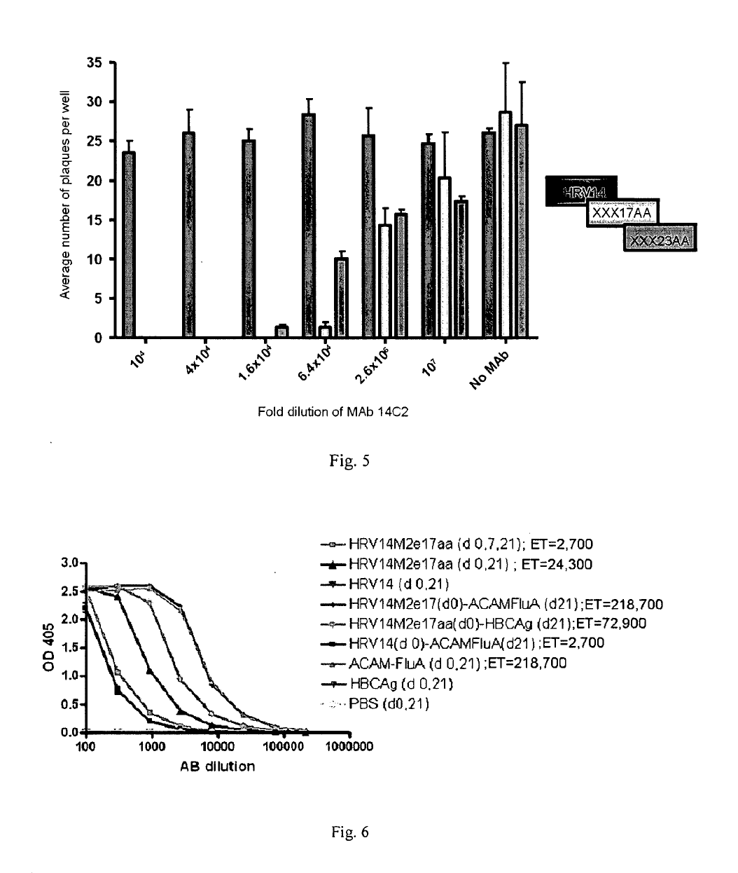

We carried out neutralization studies with the virus libraries using an anti-M2e monoclonal antibody (14C2 MAb, Abcam, Inc. Cat# ab5416). Virus neutralization can be also used as a tool to demonstrate the purity of libraries (i.e., the absence of wild type HRV14). A plaque reduction neutralization test (PRNT) demonstrated extremely high specificity and neutralizing ability of Mab 14C2 against both libraries (FIG. 5).

Both libraries were shown to be extremely susceptible to neutralization by the anti-M2e Mab (FIG. 5), while control virus (pWR1) was not neutralized, even at the lowest dilution of 1:10 of the monoclonal antibody. Fifty-percent neutralization for both libraries was observed at ˜1:2,000,000 dilution of antibody (stock concentration of 14C2 was 1 mg/ml). Such an efficient neutralization of the recombinant viruses showed that the M2e peptide presented in NimII of HRV14 is in an appropriate conformation, easily recognizable by antibodies.

### II. Identification of Stable HRV14-NimII-M2e Recombinants

After 4 passages in H1 HeLa cells, six individual clones from each library were plaque purified and, after an additional 4 passages, characterized by sequencing of the carried insert. Each library gave rise to one dominant and stably replicating viral clone. All viruses isolated from the HRV14-NimII-XXX23AA library had the same insert sequence, GHTSLLKEVETPIRNEWGSRSNDSSD (SEQ ID NO:42) with GHT as an N-terminal linker, whereas all of the viruses from the HRV14-NimII-XXX17AA library exhibited the same sequence, QPASLLTEVETPIRNEWGSR (SEQ ID NO:43), but with QPA as the N-terminal linker. All viable clones carrying the 23 amino acid insert had a substitution at position amino acid 7 from a tyrosine to lysine (position 4 in the M2e foreign insert). The clones carrying the 17 amino acid insert all contained wild type M2e sequence. These results indicate that genetically stable recombinant HRV-M2e viruses can be isolated. In further in vivo studies, the potential of HRV14-M2e (17AA) to provide protection against the PR8 strain of Influenza A was evaluated using intraperitoneal route of administration.

### III. In Vivo Study with HRV14-M2e and HRV14-HA₀ Recombinants

### A. In Vivo Experiment #1: Intraperitoneal Immunization

### 1. Experimental Design

Nine week old female Balb/c mice (8 mice per group) were primed on day 0, then boosted on day 21 by intraperitoneal administration with either 5.0×10⁶ pfu of sucrose purified HRV14-M2e (17AA; see note (4) to Table 4), 1.3×10⁷ pfu of parental HRV14, or mock (PBS) as negative controls, mixed with 100 μg of adjuvant (aluminum hydroxide) in a 500 μl volume. As a control, recombinant Hepatitis B core particles carrying 3 copies of M2e (also referred to herein as HBc-3XM2e VLPs) was used. The latter was used alone or in combination with HRV14-M2e or HRV14 for prime/boost (Table 4). To demonstrate protection, all mice were subjected to challenge with 4 LD₅₀ of influenza A/PR/8/34 (H1N1) virus on day 35. Morbidity and mortality were monitored for 21 days. To test for serum antibodies against the carried peptide, mice were bled prior to inoculation (baseline) and again on day 33. M2e-specific antibody titers in sera were determined by an established ELISA performed in microtiter plates coated with synthetic M2e peptide. Titers of M2e-specific total IgG, Ig2a, and Ig2b were determined.

### 2. Results

a. Immunogenicity
i. Total IgG in immunized animals

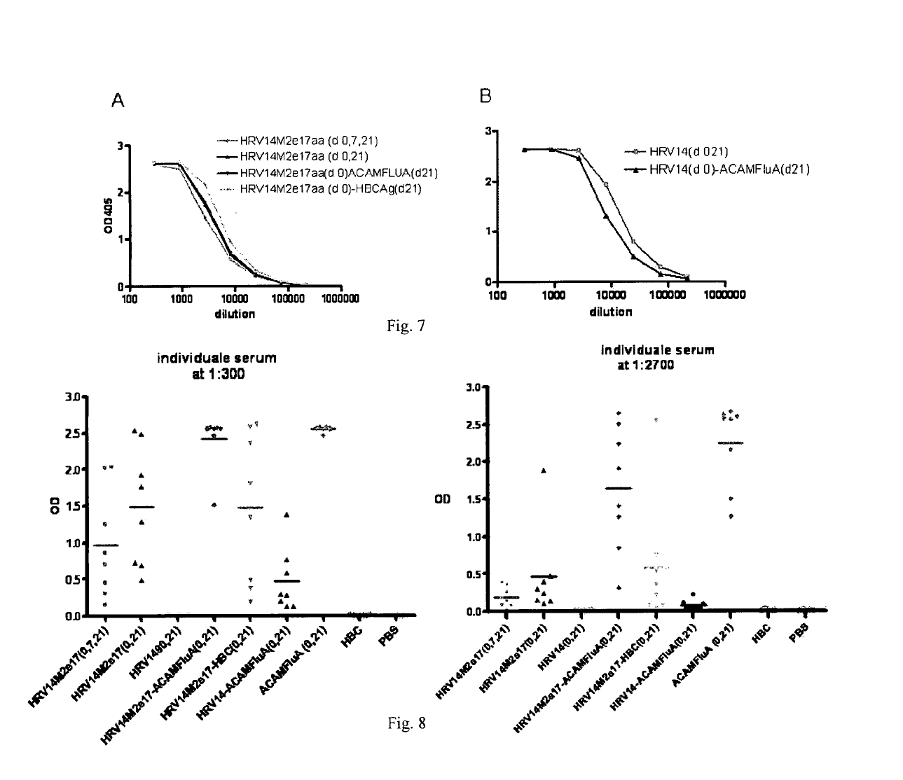

M2e-specific antibody titers were measured for each group using pooled serum samples (FIG. 6), as well as individual animal samples (FIG. 7). The results with pooled samples (FIG. 6) showed that priming with recombinant HRV14 carrying the 17 amino acid M2e sequence and boosting with hepatitis B core-M2e recombinant virus-like particles (VLPs) elicited the same levels of antibodies as two doses of the hepatitis B virus core-M2e VLPs (10 μg/dose) (end point titer (ET)=218,700). Boosting with the hepatitis B virus core-M2e VLPs elicited about a 100 times higher M2e-specific response when primed with HRV14-M2e (17AA) (group 4; ET-218,700) than with HRV14 vector (group 6; ET=2,700). Thus, the priming effect of HRV14-M2e is solely dependent on M2e insert and not on the vector.

Based on the assumption made by Arnold et al., US 2006/0088549 A1, an immunizing dose of 10⁹ pfu of HRV14 corresponds to approximately 10 μg of protein. We have roughly estimated that one immunizing dose of recombinant HRV-M2e virus represents 10 ng of protein. Taking into account differences in molecular mass and the multiplicity of subunits in the recombinant hepatitis B core particles, we speculated that one immunizing dose of HBc-M2e contained approximately 10,000 times more M2e protein than that of HRV-M2e. Comparable antibody levels using HRV vectors perhaps supports a more immunogenic presentation system, using less expensive production methodology.

The level of M2e antibodies was inversely proportional to the number of doses of HRV14-M2e (17AA). Indeed, three doses of HRV14-M2e (17AA) virus (group 1) elicited the lowest M2e-specific response (ET=2.700), whereas a two dose regimen elicited a 10 times higher response (group 2; ET=24, 300), and a one dose regimen elicited a 3 times higher response than two doses (group 5; ET=72,900). To verify whether this correlation is due to anti-vector immunity, we separately tested immune responses of all groups to the HRV14 vector (FIG. 7). All three types of administration of HRV14-M2e (17AA) (1, 2, or 3 doses) showed comparable levels of HRV14-specific responses (ET=72,900) (FIG. 7A). This argues against anti-vector immunity as a reason for decreased immune response to M2e, and suggests that a one dose administration may be sufficient.

M2e-specific ELISA analysis of individual serum samples (FIG. 8) detected the same intra-group differences as were shown with pooled samples: the average antibody levels in individual mice of groups 4 and 7 were significantly higher than for any other group studied, as was shown at two serum dilutions (1:300 and 1:2,700)

ii. IgG2a, IgG2b, and IgG1 subtypes of antibodies in immunized animals

The dominant M2-specific antibody isotype in M2e vaccinated mice was shown to be IgG2b, with some IgG2a (Jegerlehner et al., J. Immunol. 172(9):5598-5605, 2004). These two isotypes have been shown to be the most important mediators of antibody-dependent cytotoxicity (ADCC) in mice (Denkers et al., J. Immunol. 135:2183, 1985), which is believed is the major mechanism for M2e-dependent protection. In this study, we have tested pooled group and individual sera samples for IgG1, IgG2a, and IgG2b isotype titers.

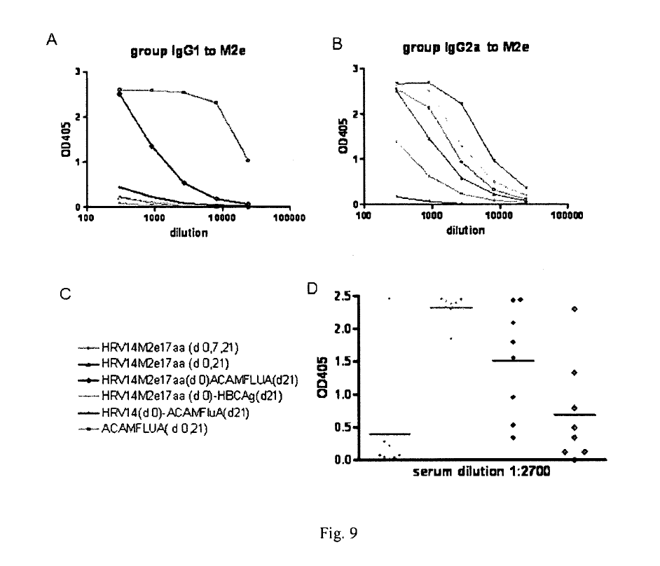

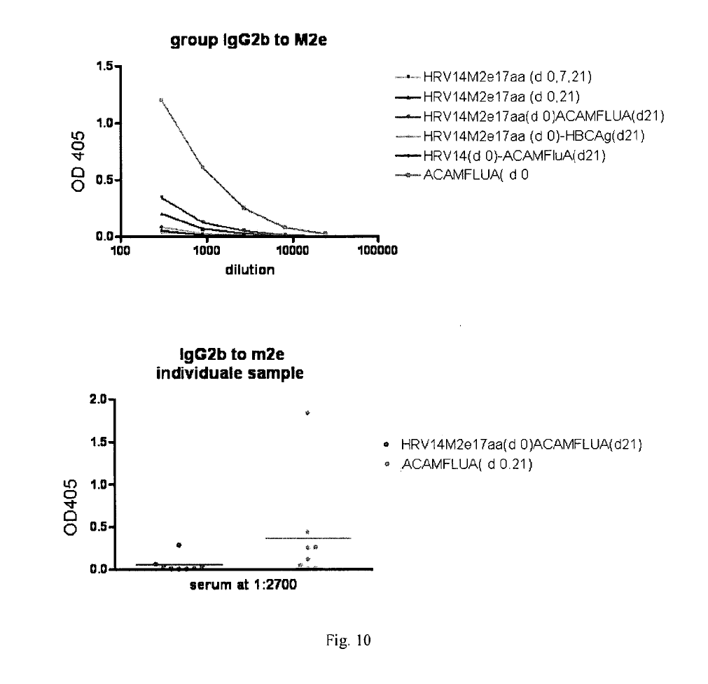

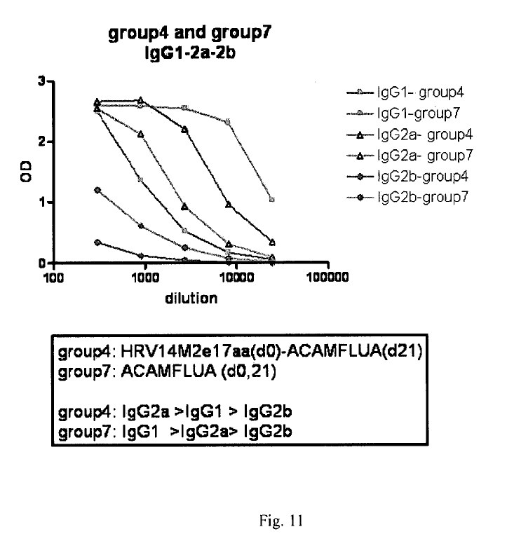

Groups 4 (prime with HRV14-M2e (17AA)/boost with hepatitis B virus core-M2e VLPs) and 7 (prime/boost with hepatitis B virus core-M2e VLPs) demonstrated the highest titers of IgG1 and IgG2a antibodies among other groups (FIG. 9). IgG1 titers were significantly higher in group 7 than in group 4 (FIGS. 9A and 9D), whereas IgG2a titers were higher in group 4 (FIGS. 9B and 9D), whereas IgG2b titers of group 7 animals were higher than in group 4 (FIG. 10). M2e-specific antibody of IgG2a isotype in mice immunized is shown in FIG. 11.

b. Morbidity and Mortality

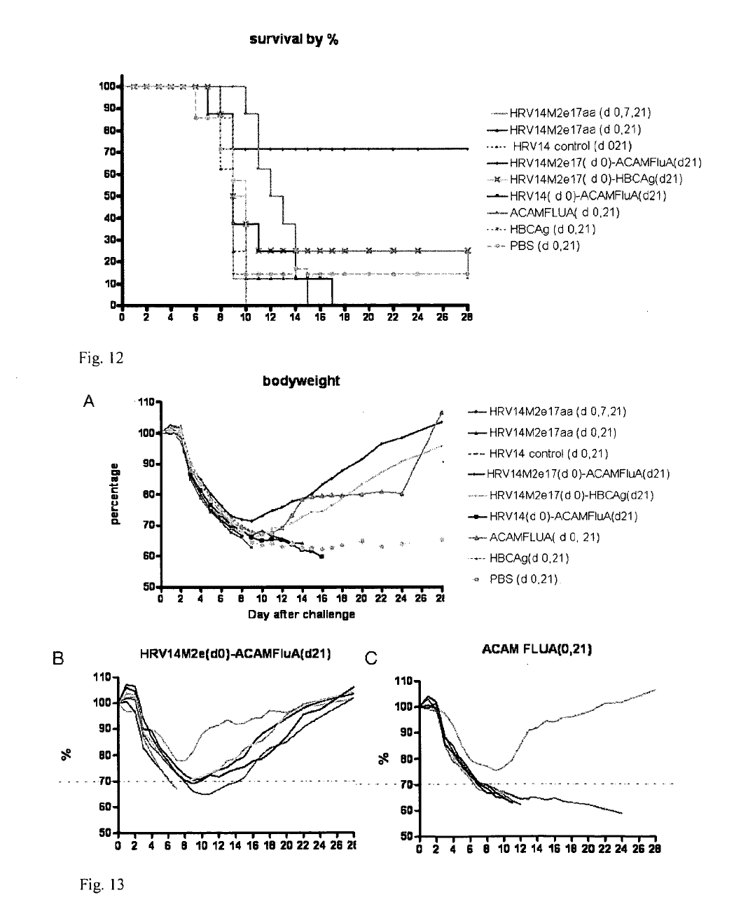

Mice were monitored for morbidity and mortality for 28 days after challenge with the PR8 strain. As is shown in FIG. 12, group 4 demonstrated the highest survival rate (80%) in comparison to all other groups studied, whereas group 7 showed no significant difference from the negative control (PBS). Group 4 was also a champion by morbidity: the body weight changes were significantly less dramatic than for all other groups (FIGS. 13A, B).

Thus, HRV14-M2e (17AA) virus is highly immunogenic and protective in mice. It is comparable to the traditional recombinant protein regimen and a combination of the two in a prime-boost regimen. The latter demonstrated a significantly different immune response than recombinant protein alone: two doses of recombinant hepatitis B virus core-M2e VLPs elicited a dominant IgG1 antibody subtype, whereas priming with HRV14-M2e (17AA) and boosting with hepatitis B virus core-M2e VLPs generated IgG2a as a dominant isotype, which was shown to be important for ADCC. Moreover, the latter group demonstrated the highest protection over all other groups.

It is important to note that, because HRV does note replicate in mice, inoculation of HRV-M2e recombinants in this model is carried out with a suitable parenteral adjuvant and mimics immunization with an inactivated vaccine. Two options may be used in humans: live recombinant HRV14-M2e virus vaccine and/or inactivated vaccine (e.g., formalin-inactivated) co-administered with a licensed parenteral adjuvant such as aluminum hydroxide (also see above).

### B. In Vivo Experiment #2. Intranasal Immunization

### 1. Viruses Used for Immunization

In this in vivo study, the potential of single insert variants HRV14-M2e (17AA), HRV14-HA₀ (19AA), or mixtures thereof, as well as double insert construct HRV14-M2e (16AA)-HA_{O} (12AA), to provide protection against mortal challenge with the PR8 strain of Influenza A was evaluated using the intranasal route of administration. The HRV14-M2e (17AA) sequence was described above. HRV14-HA₀ (19AA) contains insert NVPEKQTQGIFGAIAGFIE (SEQ ID NO:44) in NimII inserted between amino acids 159 and 160 of VP2 (NimII site). This insert was identical to the HA₀ sequence of Influenza A, except for one mutated amino acid (replacement R8Q). The latter construct does not have flanking linkers (FIG. 2C). The third construct carried insert sequence of SLLTEVETPIRNEWGSERGIFGAIAGFIE (SEQ ID NO:39) in a modified NimII site. The latter insert sequence is comprised of 16 amino acids of M2e sequence (underlined) and 12 amino acids of HA₀ sequence (bolded) of Influenza A/H3. These two sequences are separated by a 1 amino acid linker (E). The insertion site (NimII) of this third construct was modified: 3 amino acids 160-162 of VP2 were replaced by proline (FIG. 2C). Virus growth was shown to be comparable with HRV14, stably maintaining inserts over 9 sequential passages.

2. Experimental Design The purpose of this animal experiment was to check to see if one dose of recombinant rhinovirus chimeras given intranasally, with or without adjuvant, elicits a protective immune response against mortal challenge with influenza A/PR/8/34 (H1N1) strain comparable to 2 doses of HBc-3XM2e VLPs.

The experimental design is shown in Table 5. Briefly, nine week old female Balb/c mice (10 mice per group) were immunized by intranasal administration on day 0 with either HBc-M2e VLPs (groups 1 and 2), HRV14-M2e (17AA) (groups 3 and 4), HRV14 (group 5), HRV14-HA₀ (19AA) (group 6), HRV14-HA₀ (19AA) mixed with HRV14-M2e (17AA) (groups 7 and 8) or PBS control (group 9), or HRV14-M2e (16AA)-HA₀ (12AA). Groups 1, 3, 5, 6, 7, and 13 were administered with 5 μg of Heat-Labile Toxin of E. coli (LT) adjuvant, while groups 2, 4, and 8 were administered without adjuvant (Table 5). The administration volume was 50 μl. Groups 1 and 2 were boosted on day 21 by intranasal administration with 10 μg HBc-3XM2e with LT adjuvant in a 50 μl administration volume.

To validate another adjuvant (chitin), mice were immunized via the intranasal route with either HBc-M2e VLPs (group 10), HRV14-M2e (17AA) (group 11), or HRV14 (group 12) mixed with 25 μg of chitin in a 50 μl administration volume. Group 10 was boosted on day 21 by intranasal administration with 10 μg HBc-3XM2e with the same adjuvant in a 50 μl administration volume.

To demonstrate protection, all mice were subjected to challenge with 4 LD₅₀ of influenza A/PR/8/34 (H1N1) virus on day 35. Morbidity and mortality were monitored for 21 days. To test for serum antibodies against the carried peptide, mice were bled prior to inoculation (baseline) and again on day 33. M2e- and HA_{O}-specific antibody titers in sera were determined by an established ELISA performed in microtiter plates coated with synthetic M2e and HA₀ peptides. Titers of M2e-specific total IgG, Ig2a, and Ig2b were determined.

### 3. Results

a. Immunogenicity

i. M2e- and HA₀-Specific Antibody Titers

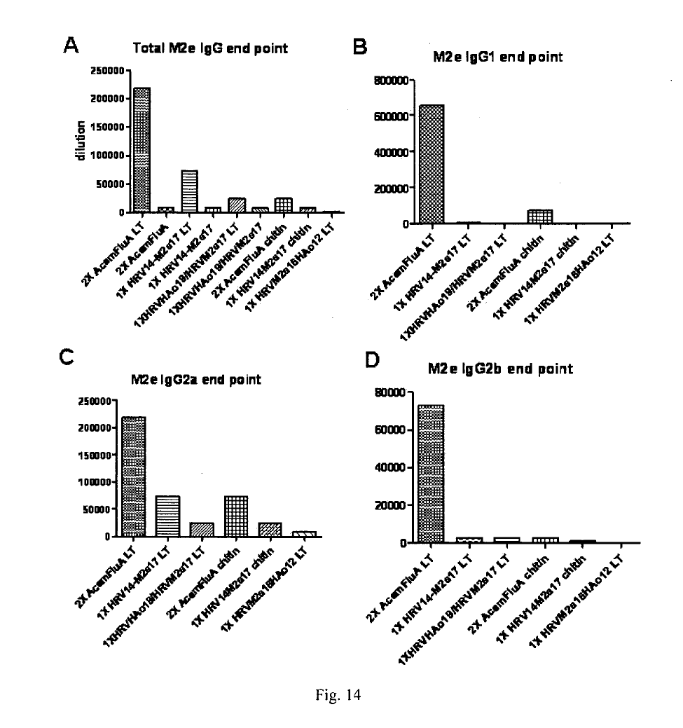

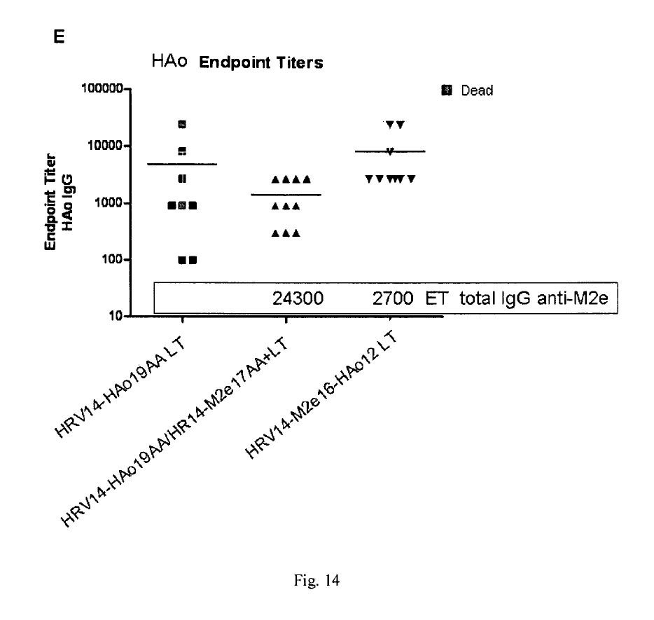

Antibody M2e titers were measured for each group using pooled serum samples (FIG. 14 A-D). One dose of recombinant HRV14 carrying the 17 amino acid M2e peptide elicited comparable levels of total IgG to two doses of the hepatitis B virus core-M2e recombinant VLPs (10 ug/dose) (end point titers (ET) for HBc-M2e and one HRV14-M2e (17AA) were 218,700 and 72,900 respectively (FIG. 14A)). Adjuvant (LT) played a significant role in protection provided with both HBc- and HRV-based vaccines: immune response in groups with no LT was on average ten fold less than in LT-groups. Chitin adjuvant groups demonstrated >100-1000 fold less M2e response. A two-fold reduction in HRV14-M2e virus load (group 7) had a 3 fold reducing effect on total IgG titer (group 7; ET=24,300 vs. 72,900 for group 3).

One dose of HRV14-M2e generated the second highest level of IgG2a (FIG. 14C; ET=72, 900 vs. 218.700 for HBc-M2e). The highest titers IgG2b (FIG. 14B) and IgG1 (FIG. 14D) were demonstrated for two doses of the hepatitis B core-M2e VLPs.

Antibody HA₀ titers were measured for groups 6, 7, and 13 using individual serum samples (FIG. 14E). Geometric means of end point titers were amounted to 4750 for group 6 (HRV14-HA₀ (19 AA) with LT), 1440 for group 7 (mix of HRV14-HA₀ (19AA) with HRV14-M2e (17AA) with LT), and 9200 for group 13 (HRV14-M2e (16AA)-HA₀ (12AA) with LT). The highest HA₀ response in group 13 could be explained by the presence of the wild type HA₀ sequence of A/H3, while recombinant chimeras in groups 6 and 7 carried mutated version of the HA₀ cleavage site (R8Q). The arginine residue at position 8 of HA₀ was shown previously to be critical for protection, as well as was demonstrated as one of three binding sites for protective monoclonal antibodies (Bianchi et al., J. Virol. 79:7380-7388, 2005). M2e pooled sample titers for groups 7 and 13 are shown in FIG. 14E, in a boxed area to emphasize that the M2e response in group 13 was low (ET=2700; compare with ET=7,200 for group 5; HRV14-M2e with no adjuvant), which showed that the M2e epitope in the HRV14-M2e (16AA)-HA₀ (12AA) chimera was not immunogenic, possibly due to its poor exposure on the viral surface. Therefore, high immunogenicity/protection (see below) of this variant should be attributed to HA₀, but not to the M2e epitope.

b. Morbidity and Mortality

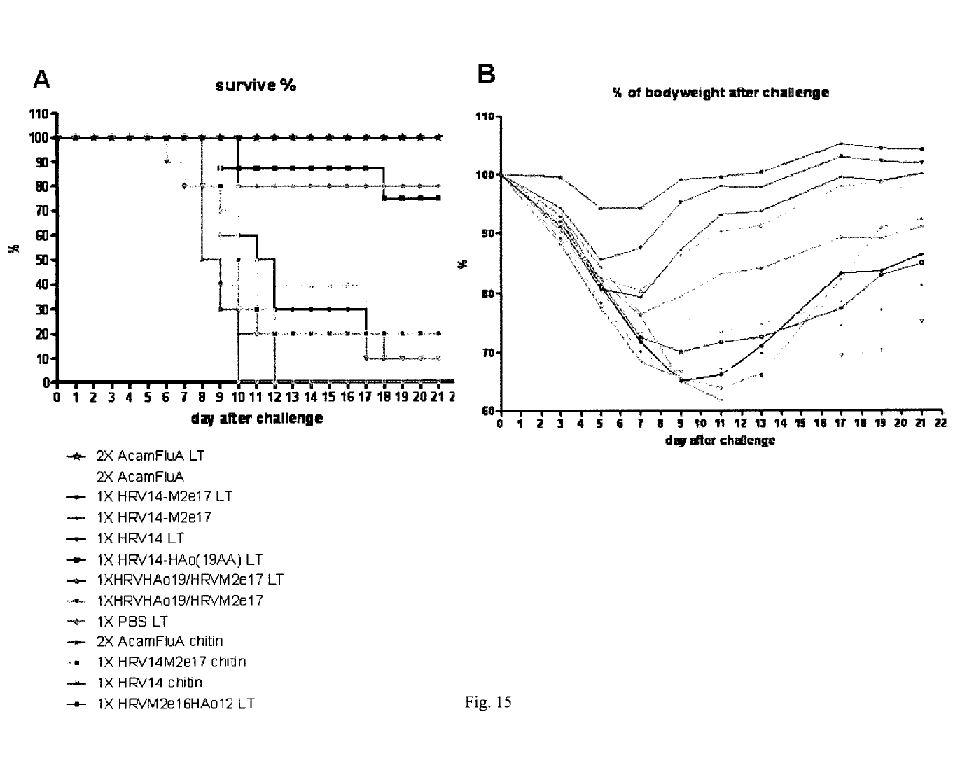

Mice were monitored for morbidity for 21 days after mortal challenge with the PR8 strain (FIG. 15B). One dose of either HRV14-M2e (group 3) or HRV14-M2e (16AA)-HA_{O} (12AA) (group 13) provided comparable protection from disease as two doses of HBc-M2e VLPs (group 1). All mice of these groups survived mortal challenge (FIG. 15A). Taking into account the difference in immunogenicity between these two groups (see above), one could strongly suggest that protection in these two groups is provided by different epitopes: M2e for group 3 (HRV14-M2e) and HA₀ for group 13 (HRV14-M2e (16AA)-HA₀ (12AA)).

One dose of HRV14 carrying a mutated HA_{O} cleavage site (group 6) demonstrated high morbidity, similar to 2 mice that survived in control HRV14 group 5. This correlates with a lower (75%) survival rate. One dose with a mixture of HRV14-M2e (17AA) and HRV14-HA₀ (19AA) viruses (group 7) showed slightly higher morbidity than the HRV14-M2e (17AA) group, which was correlated with a reduction in viral load in immunization doses by half (FIG. 15B). However, all mice of group 7 were 100% protected against mortal influenza challenge. The latter protection should be attributed to M2e, rather than the HA₀ epitope, since doubling of HRV14-HA_{O} (19AA) (group 6) resulted, as mentioned above, in a 75% survival rate.

Adjuvant played a significant role in protection: all mice in “no adjuvant” groups died on days 9-10 after challenge (FIG. 15A). LT provided better protection than chitin: all mice in the HRV14-M2e (17AA)+chitin (group 11) died, while two doses of HRV-3XM2e VLPs+chitin (group 10) resulted in 80% protection (FIG. 15A).

Thus, we demonstrated that one dose of HRV14 recombinant chimeras carrying either the HA₀ or M2e universal protective epitopes provided 100% protection against mortal influenza A challenge when administered via the intranasal route. This protection was comparable to that provided by two-dose administration of HBc-3XM2e VLPs via the same route.

### IV. Influenza Mouse Challenge Model

The protective efficacy of vaccine candidates can be tested in a mouse influenza challenge model using appropriate virus strains. The prototype influenza challenge strain used in the studies described herein is mouse-adapted strain A/PR/8/34 (H1N1). The virus was obtained from the American Type Culture Collection (catalog number VR-1469, lot number 2013488) and adapted to in vivo growth by serial passage in Balb/c mice. For mouse passage, virus was inoculated intranasally and lung tissue homogenates were prepared 3 days later. The homogenate was blind-passaged in additional mice through passage 5. An additional passage was used to prepare aliquots of lung homogenate that serve as the challenge stock.

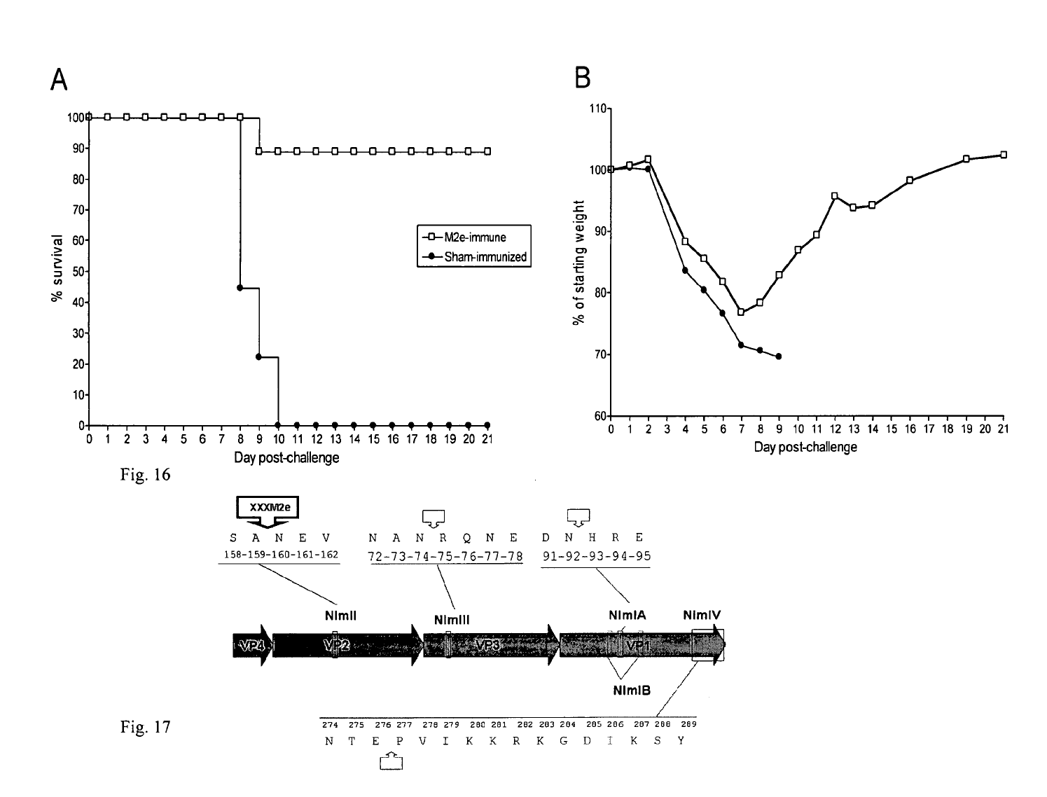

For challenge of mice, virus is delivered intranasally in a volume of 50 μL. The mice are anesthetized during inoculation to inhibit the gag reflex and allow passage of the virus into the lungs. Mice infected with a lethal dose of virus rapidly lose weight and most die 7-9 days after inoculation. The median lethal dose (LD₅₀) of mouse-adapted A/PR/8/34 virus was determined to be 7.5 plaque-forming units (pfu) in adult Balb/c mice. Results for a typical protection experiment are shown in FIG. 16. Groups of 10 mice were either sham-immunized with aluminum hydroxide adjuvant or immunized with 10 μg of influenza M2e peptide immunogen mixed with aluminum hydroxide. The immunogen consisted of hepatitis B core protein VLPs expressing an M2e peptide. The mice were immunized twice at 3-week intervals and challenged intranasally 4 weeks later with 4 LD₅₀ of mouse-adapted A/PR/8/34 virus. All mice in the sham-immunized group died by the 10^{th} day after challenge, while only 1 mouse died in the immunized group. Loss in weight occurred after challenge in both groups, but was greater in the sham-immunized group.

Other influenza virus strains can be similarly adapted to grow in mouse lungs. In some cases, strains may be used without in vivo adaptation or may not become sufficiently pathogenic even after serial lung passage. In this case, rather than measuring morbidity and mortality, we can measure virus replication in lung and nasal turbinate tissues. Tissues are harvested 3 days after challenge, disrupted by sonication in 1 ml of tissue culture medium, and titrated for virus concentration by plaque or TCID₅₀ assay.

In addition to the challenge model described above, the invention also includes use of animal model systems such as those described by Bartlett et al., Nature Medicine 14(2):199-204, 2008. In one example, the invention may employ a mouse, such as a BALB/c mouse, expressing a mouse-human intercellular adhesion molecule-1 (ICAM-1) chimera, which can be generated according to the methods described by Bartlett. As is known in the art, ICAM-1 is the cellular receptor of 90% of human rhinoviruses, which do not bind to mouse ICAM-1. As taught by Bartlett, human rhinoviruses bind to chimeras including the rhinovirus-binding extracellular domains 1 and 2 of human ICAM-1, in the context of transgenic mice. This provides a useful system for the study of live rhinovirus vectors, such as those described herein. The invention therefore includes screening for and testing of vaccine candidates in such mouse models.

TABLE 1
List of examples of pathogens from which
epitopes/antigens/peptides can be derived

VIRUSES:
Flaviviridae
Yellow Fever virus
Japanese Encephalitis virus
Dengue virus, types 1, 2, 3, and 4
West Nile Virus
Tick Borne Encephalitis virus
Hepatitis C virus (e.g., genotypes 1a, 1b,
2a, 2b, 2c, 3a, 4a, 4b, 4c, and 4d)
Papoviridae:
Papillomavirus
Retroviridae
Human Immunodeficiency virus, type I
Human Immunodeficiency virus, type II
Simian Immunodeficiency virus
Human T lymphotropic virus, types I & II
Hepnaviridae
Hepatitis B virus
Picornaviridae
Hepatitis A virus
Rhinovirus
Poliovirus
Herpesviridae:
Herpes simplex virus, type I
Herpes simplex virus, type II
Cytomegalovirus
Epstein Barr virus
Varicella-Zoster virus
Togaviridae
Alphavirus
Rubella virus
Paramyxoviridae
Respiratory syncytial virus
Parainfluenza virus
Measles virus
Mumps virus
Orthomyxoviridae
Influenza virus
Filoviridae
Marburg virus
Ebola virus
Rotoviridae
Rotavirus
Coronaviridae
Coronavirus
Adenoviridae
Adenovirus
Rhabdoviridae
Rabiesvirus
BACTERIA:
Enterotoxigenic E. coli
Enteropathogenic E. coli
Campylobacter jejuni
Helicobacter pylori
Salmonella typhi
Vibrio cholerae
Clostridium difficile
Clostridium tetani
Streptococccus pyogenes
Bordetella pertussis
Neisseria meningitides
Neisseria gonorrhoea
Legionella neumophilus
Clamydial spp.
Haemophilus spp.
Shigella spp.
PARASITES:
Plasmodium spp.
Schistosoma spp.
Trypanosoma spp.
Toxoplasma spp.
Cryptosporidia spp.
Pneumocystis spp.
Leishmania spp.

TABLE 2
Examples of select antigens from listed viruses

| VIRUS | ANTIGEN |
|---|---|
| Flaviviridae | |
| Yellow Fever virus | Nucleocapsid, M & E glycoproteins |
| Japanese Encephalitis virus | ″ |
| Dengue virus, types 1, 2, 3 & 4 | ″ |
| West Nile Virus | ″ |
| Tick Borne Encephalitis virus | ″ |
| Hepatitis C virus | Nucleocapsid, E1 & E2 glycoproteins |
| Papoviridae: | |
| Papillomavirus | L1 & L2 capsid protein, E6 |
| | & E7 transforming protein (oncogenes) |
| Retroviridae | |
| Human Immunodeficiency | gag, pol, vif, tat, vpu, env, nef |
| virus, type I | |
| Human Immunodeficiency | ″ |
| virus, type II | |
| Simian Immunodeficiency | ″ |
| virus | |
| Human T lymphotropic virus, | gag, pol, env |
| types I & II | |

TABLE 3
Examples of B and T cell epitopes from listed viruses/antigens

| VIRUS | ANTIGEN | EPITOPE | LOCATION | SEQUENCE (5′-3′) |
|---|---|---|---|---|
| Flaviviridae | | | | |
| Hepatitis C | Nucleocapsid | CTL | 2-9 | STNPKPQR |
| | | | | (SEQ ID NO: 45) |
| | | | 35-44 | YLLPRRGPRL |
| | | | | (SEQ ID NO: 46) |
| | | | 41-49 | GPRLGVRAT |
| | | | | (SEQ ID NO: 47) |
| | | | 81-100 | YPWPLYGNEGCGWAGWLLSP |
| | | | | (SEQ ID NO: 48) |
| | | | 129-144 | GFADLMGYIPLVGAPL |
| | | | | (SEQ ID NO: 49) |
| | | | 132-140 | DLMGYIPLV |
| | | | | (SEQ ID NO: 50) |
| | | | 178-187 | LLALLSCLTV |
| | | | | (SEQ ID NO: 51) |
| | E1 g1ycoprotein | CTL | 231-250 | REGNASRCWVAVTPTVATRD |
| | | | | (SEQ ID NO: 52) |
| | E2 glycoprotein | CTL | 686-694 | STGLIHLHQ |
| | | | | (SEQ ID NO: 53) |
| | | | 725-734 | LLADARVCSC |
| | | | | (SEQ ID NO: 54) |
| | | | 489-496 | CWHYPPRPCGI |
| | | | | (SEQ ID NO: 55) |
| | | | 569-578 | CVIGGVGNNT |
| | | | | (SEQ ID NO: 56) |
| | | | 460-469 | RRLTDFAQGW |
| | | | | (SEQ ID NO: 57) |
| | | | 621-628 | TINYTIFK |
| | | | | (SEQ ID NO: 58) |
| | | B cell | 384-410 | ETHVTGGNAGRTTAGLVGLL |
| | | | | TPGAKQN |
| | | | | (SEQ ID NO: 59) |
| | | | 411-437 | IQLINTNGSWHINSTALNCNESLNTGV |
| | | | | (SEQ ID NO: 60) |
| | | | 441-460 | LFYQHKFNSSGCPERLASCR |
| | | | | (SEQ ID NO: 61) |
| | | | 511-546 | PSPVVVGTTDRSGAPTYSWGANDTD |
| | | | | FVLNNTRPPL |
| | | | | (SEQ ID NO: 62) |
| | | T helper | 411-416 | IQLINT |
| | | | | (SEQ ID NO: 63) |
| Papoviridae | | | | |
| HPV 16 | E7 | T helper | 48-54 | DRAHYNI |
| | | | | (SEQ ID NO: 64) |
| | | CTL | 49-57 | RAHYNIVTF |
| | | | | (SEQ ID NO: 65) |
| | | B cell | 10-14 | EYMLD |
| | | | | (SEQ ID NO: 66) |
| | | | 38-41 | IDGP |
| | | | | (SEQ ID NO: 67) |
| | | | 44-48 | QAEPD |
| | | | | (SEQ ID NO: 68) |
| HPV 18 | E7 | T helper | 44-55 | VNHQHLPARRA |
| | | | | (SEQ ID NO: 69) |
| | | | 81-90 | DDLRAFQQLF |
| | | | | (SEQ ID NO: 70) |

TABLE 4
Immunization groups (Intraperitoneal Study)

| | Number | | | | |
|---|---|---|---|---|---|
| Group | of | | | | Dosing |
| number | animals | Prime | Boost | Adjuvant | (days) |
| 1 | 8 | HRV14- | HRV14- | Alum | 0, 7, 21 |
| | | M2e(17AA) | M2e(17AA) | | |
| 2 | 8 | HRV14- | HRV14- | Alum | 0, 21 |
| | | M2e(17AA) | M2e(17AA) | | |
| 3 | 8 | HRV14 | HRV14 | Alum | 0, 21 |
| 4 | 8 | HRV14- | HBc-M2e | Alum | 0, 21 |
| | | M2e(17AA) | | | |
| 5 | 8 | HRV14- | HBcAg | Alum | 0, 21 |
| | | M2e(17AA) | | | |
| 6 | 8 | HRV14 | HBc-M2e | Alum | 0, 21 |
| 7 | 8 | HBc-M2e | HBc-M2e | Alum | 0, 21 |
| 8 | 8 | HBcAg | HBcAg | Alum | 0, 21 |
| 9 | 8 | PBS | PBS | Alum | 0, 21 |

Notes for Table 4:
(1) HBc-M2e is based on Hepatitis B core antigen (HBc) carrying three copies of 23 AA M2-e peptide; the dose = 10 μg per mouse.
(2) HBcAg is a “naked” HBc antigen; used as carrier control for HBc-M2e; the dose = 10 μg per mouse
(3) HRV14 is “wild type” HRV14 produced from pWR3.26 infectious clone (ATCC); used as a carrier control for HRV14-M2e (17AA).
(4) HRV14M2e (17AA) is HRV14 virus carrying QPASLLTEVETPIRNEWGSR (SEQ ID NO: 43) sequence between amino acid 159 and 160 of VP2 (NimII site). The first three amino acids (QPA) of this insert represent a unique linker selected from HRV14M2eXXX (17AA) library, as described earlier.
(5) Adjuvant - alum was used in all immunizations.
(6) All groups were immunized by intraperitoneal administration.

TABLE 5
Immunization groups (Intranasal Study)

| | Number | | | |
|---|---|---|---|---|
| Group | of | Prime | Boost | |
| Number | Animals | (Day 0) | (Day 21) | Adjuvant |
| 1 | 10 | HBc-M2e | HBc-M2e | LT |
| | | VLPs | VLPs | (5 μg) |
| | | (10 μg) | (10 μg) | |
| 2 | 10 | HBc-M2e | HBc-M2e | |
| | | VLPs | VLPs | |
| | | (10 μg) | (10 μg) | |
| 3 | 10 | HRV14-M2e | | LT |
| | | (17AA) (~10⁸ pfu) | | (5 μg) |
| 4 | 10 | HRV14-M2e | | |
| | | (17AA) (~10⁸ pfu) | | |
| 5 | 10 | HRV14 (~10⁸ pfu) | | LT |
| | | | | (5 μg) |
| 6 | 10 | HRV14-HA₀ | | LT |
| | | (19AA) (~10⁸ pfu) | | (5 μg) |
| 7 | 10 | HRV14-HA₀ | | LT |
| | | (19AA) + HRV14- | | (5 μg) |
| | | M2e (17AA) | | |
| | | (~0.5 × 10⁸ pfu each) | | |
| 8 | 10 | HRV14-HA₀ | | |
| | | (19AA) + HRV14- | | |
| | | M2e (17AA) | | |
| | | (~0.5 × 10⁸ pfu each) | | |
| 9 | 10 | PBS | | LT |
| | | | | (5 μg) |
| 10 | 10 | HBc-M2e | HBc-M2e | Chitin |
| | | VLPs | VLPs | (25 μg) |
| | | (10 μg) | (10 μg) | |
| 11 | 10 | HRV14-M2e | | Chitin |
| | | (17AA) (~10⁸ pfu) | | (25 μg) |
| 12 | 10 | HRV14 (~10⁸ pfu) | | Chitin |
| | | | | (25 μg) |
| 13 | 10 | HR-M2e- HA₀ | | LT |
| | | 12aa (~10⁸ pfu) | | (5 μg) |

Notes for Table 5:
(1) HBc-M2e is based on Hepatitis B core antigen (HBc) carrying three copies of 23 AA M2-e peptide; the dose = 10 μg per mouse.
(2) HRV14 is “wild type” HRV14 produced from pWR3.26 infectious clone (ATCC); used as a carrier control for HRV14-M2e (17AA).
(3) HRV14M2e (17AA) is HRV14 virus carrying QPASLLTEVETPIRNEWGSR (SEQ ID NO: 43) sequence between amino acids 159 and 160 of VP2 (NimII site). The first three amino acids (QPA) of this insert represent a unique linker selected from HRV14M2eXXX (17AA) library as described earlier
(4) HRV14-HA₀ (11AA) contains insert GIFGAIAGFIE (SEQ ID NO: 71) in NimII inserted between amino acid 159 and 160 of VP2 (NimII site). This construct does not have flanking linkers.
(5) Adjuvant - alum was used in all immunizations; LT = Heat-Labile Toxin of E. coli.
(6) All groups were immunized by intranasal administration.
(7) Groups 3, 4, 5, and 6 were immunized with correspondent viruses at 10⁸ pfu per dose; group 8 was immunized with mix of HRV14-M2e (17AA) and HRVI4-HA₀ at 5 × 10⁷ pfu per dose for each virus.

TABLE 6
Extracellular Part of M2 Protein of Human
Influenza A Strains

| Virus strain (subtype) | |
|---|---|
| A/WS/33 (H1N1) | SLLTEVETPIRNEWGCRCNDSSD¹ |
| A/WSN/33 (H1N1) | SLLTEVETPIRNEWGCRCNDSSD |
| A/NWS/33 (H1N1) | SLLTEVETPIRNEWGCRCNDSSD |
| A/PR/8/34 (H1N1) | SLLTEVETPIRNEWECRCNGSSD² |
| A/Fort Monmouth/1/47 | SLLTEVETPTKNEWGCRCNDSSD³ |
| (H1N1) | |
| A/fort Warren/1/50 (H1N1) | SLLTEVETPIRNEWGCRCNDSSD |
| A/JapanxBellamy/57 (H2N1) | SLLTEVETPIRNEWGCRCNDSSD |
| A/Singapore/1/57 (H2N2) | SLLTEVETPIRNEWGCRCNDSSD |
| A/Leningrad/134/57 (H2N2) | SLLTEVETPIRNEWGCRCNDSSD |
| A/Ann Harbor/6/60 (H2N2) | SLLTEVETPIRNEWGCRCNDSSD |
| A/NT/60/68 (hxNy) | SLLTEVETPIRNEWGCRCNDSSD |
| A/Aichi/2/68 (H3N2) | SLLTEVETPIRNEWGCRCNDSSD |
| A/Korea/426/68 (H2N2) | SLLTEVETPIRNEWGCRCNDSSD |
| A/Hong Kong/1/68 (H3N2) | SLLTEVETPIRNEWGCRCNDSSD |
| A/Udorn/72 (H3N2) | SLLTEVETPIRNEWGCRCNDSSD |
| A/Port Chalmers/73 (H3N2) | SLLTEVETPIRNEWGCRCNDSSD |
| A/USSR/90/77 (H1N1) | SLLTEVETPIRNEWGCRCNDSSD |
| A/Bangkok/1/79 | SLLTEVETPIRNEWGCRCNDSSD |
| A/Philippines/2/82/BS | SLLTEVETPIRNEWGCRCNGSSD⁴ |
| (H3N2) | |
| A/NY/83 (H3N2) | SLLTEVETPIRNEWGCRCNDSSD |
| A/Memphis/8/88 (H3N2) | SLLTEVETPIRNEWGCRCNDSSD |
| A/Beijing/353/89 (H3N2) | SLLTEVETPIRNEWGCRCNDSSD |
| A/Guangdong/39/89 (H3N2) | SLLTEVETPIRNEWGCRCNDSSD |
| A/Kitakyushu/159/93 (H3N2) | SLLTEVETPIRNEWGCRCNDSSD |
| A/Hebei/12/93 (H3N2) | SLLTEVETPIRNEWGCRCNDSSD |
| A/Aichi/69/94 (H3N2) | SLLTEVETPIRNEWECRCNGSSD² |
| A/Saga/447/94 (H3N2) | SLLTEVETPIRNEWECRCNGSSD² |
| A/Sendai/c182/94 (H3N2) | SLLTEVETPIRNEWGCRCNDSSD |
| A/Akita/1/94 (H3N2) | SLLTEVETPIRNEWGCRCNDSSD |
| A/Sendai/c384/94 (H3N2) | SLLTEVETPIRNEWGCRCNDSSD |
| A/Miyagi/29/95 (H3N2) | SLLTEVETPIRNEWGCRCNDSSD |
| A/Charlottesville/31/95 | SLLTEVETPIRNEWGCRCNDSSD |
| A/Akita/1/95 (H3N2) | SLLTEVETPIRNEWECRCNGSSD² |
| A/Shiga/20/95 (H3N2) | SLLTEVETPIRNEWGCRCNDSSD |
| A/Tochigi/44/95 (H3N2) | SLLTEVETPIRNEWECRCNGSSD² |
| A/Hebei/19/95 (H3N2) | SLLTEVETPIRNEWECRCNGSSD² |
| A/Sendai/c373/95 (H3N2) | SLLTEVETPIRNEWECRCNGSSD² |
| A/Niigata/124/95 (H3N2) | SLLTEVETPIRNEWECRCNGSSD² |
| A/Ibaraki/1/95 (H3N2) | SLLTEVETPIRNEWECRCNGSSD² |
| A/Kagoshima/10/95 (H3N2) | SLLTEVETPIRNEWECRCNGSSD |
| A/Gifu/2/95 (H3N2) | SLLTEVETPIRNEWECRCNGSSD² |
| A/Osaka/c1/95 (H3N2) | SLLTEVETPIRNEWECRCNGSSD² |
| A/Fukushima/140/96 (H3N2) | SLLTEVETPIRNEWGCRCNDSSD |
| A/Fukushima/114/96 (H3N2) | SLLTEVETPIRNEWGCRCNDSSD |
| A/Niigata/137/96 (H3N2) | SLLTEVETPIRNEWGCRCNDSSD |
| A/Hong Kong/498/97 (H3N2) | SLLTEVETPIRNEWGCRCNDSSD |
| A/Hong Kong/497/97 (H3N2) | SLLTEVETPIRNEWGCRCNDSSD |
| A/Hong Kong/470/97 (H1N1) | SLLTEVETPIRNEWGCRCNDSSD |
| A/Shiga/25/97 (H3N2) | SLLTEVETPIRNEWGCRCNDSSD |
| A/Hong Kong/427/98 (H1N1) | SLLTEVETPIRNEWECRCNDSSD⁵ |
| A/Hong Kong/1143/99 | SLLTEVETPIRNEWGCRCNDSSD |
| (H3N2) | |
| A/Hong Kong/1144/99 | SLLTEVETPIRNEWGCRCNDSSD |
| (H3N2) | |
| A/Hong Kong/1180/99 | SLLTEVETPIRNEWGCRCNDSSD |
| (H3N2) | |
| A/Hong Kong/1179/99 | SLLTEVETPIRNEWGCRCNDSSD |
| (H3N2) | |

¹All sequences in this table correspond to SEQ ID NO: 36, unless otherwise indicated
²SEQ ID NO: 72
³SEQ ID NO: 73
⁴SEQ ID NO: 74
⁵SEQ ID NO: 75

TABLE 7
Influenza A virus CTL Epitopes of the Nucleoprotein

| Amino Acid Positions (ref.) | Host | MHC restriction |
|---|---|---|
| 44-52 (ref. 14) | Human | HLA-A1 |
| 50-63 (ref. 3) | Mouse (CBA) | H-2Kk |
| 91-99 (ref. 13) | Human | HLA-Aw68 |
| 147-158 (ref. 5) | Mouse (Balb/c) | H-2Kd |
| 265-273 (ref. 14) | Human | HLA-A3 |
| 335-349 (ref. 1) | Human | HLA-B37 |
| 335-349 (ref. 2) | Mouse | HLA-B37 |
| 365-380 (ref. 2) | Mouse | H-2Db |
| 366-374 (ref. 9) | Mouse (C57B1/6) | H-2Db |
| 380-388 (ref. 16) | Human | HLA-B8 |
| 383-391 (ref. 16) | Human | HLA-B27 |

TABLE 8
Influenza A virus T helper Epitopes of the Nucleoprotein

| Amino Acid Positions (ref.) | Host | MHC restriction |
|---|---|---|
| 55-69 (ref. 8) | Mouse (Balb/c) | H-2Kd |
| 182-205 (ref. 11) | Human | |
| 187-200 (ref. 8) | Mouse (CBA) | H-2Kk |
| | Mouse (Balb/c) | H-2Kd |
| 216-229 (ref. 8) | Mouse (Balb/c) | H-2Kd |
| 206-229 (ref. 11) | Human | HLA-DR1, HLA-DR2 en |
| | | HLA-DRw13 |
| 260-283 (ref. 8) | Mouse (CBA) | H-2Kk |
| | Mouse (C57B1/6) | H-2Db |
| | Mouse (B10.s) | H-2s |
| 297-318 (ref. 11) | Human | |
| 338-347 (ref. 16) | Human | HLA-B37 |
| 341-362 (ref. 11) | Human | |
| 413-435 (ref. 8) | Mouse (C57B1/6) | H-2Db |

TABLE 9
Influenza A Virus T cell Epitopes of Other Viral Proteins

| Peptide | Host | T cell type | MHC restriction |
|---|---|---|---|
| PB1 (591-599) (ref. 14) | Human | CTL | HLA-A3 |
| HA (204-212) (ref. 16) | Mouse | CTL | H-2Kd |
| HA (210-219) (ref. 16) | Mouse | CTL | H-2Kd |
| HA (259-266) (ref. 16) | Mouse | CTL | H-2Kk |
| HA (252-271) (ref. 7) | Mouse | CTL | H-2Kk |
| HA (354-362) (ref. 16) | Mouse | CTL | H-2Kk |
| HA (518-526) (ref. 16) | Mouse | CTL | H-2Kk |
| HA (523-545) (ref. 10) | Mouse | CTL | |
| NA (76-84) (ref. 16) | Mouse | CTL | H-2Dd |
| NA (192-201) (ref. 16) | Mouse | CTL | H-2Kd |
| M1 (17-29) (ref. 6) | Human | T helper | HLA-DR1 |
| M1 (56-68) (ref. 4) | Human | CTL | HLA-A2 |
| M1 (58-66) (ref. 12) | Human | CTL | HLA-A2 |
| M1 (128-135) (ref. 15) | Human | CTL | HLA-B35 |
| NS1 (122-130) (ref. 15) | Human | CTL | HLA-A2 |
| NS1 (152-160) (ref. 16) | Mouse | CTL | H-2Kk |

### REFERENCES FOR TABLES 7-9

(1) McMichael et al., J. Exp. Med. 164:1397-1406, 1986.
(2) Townsend et al., Cell 44:959-968, 1986.
(3) Bastin et al., J. Exp. Med. 165:1508-1523, 1987.
(4) Gotch et al., Nature 326:881-882, 1987.
(5) Bodmer et al., Cell 52:253-258, 1988.
(6) Ceppelini et al., Nature 339:392-394, 1989.
(7) Sweetser et al., Nature 342:180-182, 1989.
(8) Gao et al., J. Immunol. 143:3007-3014, 1989.
(9) Rotzschke et al., Nature 348:252-254, 1990.
(10) Milligan et al., J. Immunol. 145:3188-3193, 1990.
(11) Brett et al., J. Immunol. 147:984-991, 1991.
(12) Bednarek et al., J. Immunol. 147:4047-4053, 1991.
(13) Cerundolo et al., Proc. Roy. Soc. Lond. Series B boil. Sci. 244:169-177, 1991.
(14) DiBrino et al., J. Immunol. 151:5930-5935, 1993.
(15) Dong et al., Eur. J. Immunol. 26:335-339, 1996.
(16) Parker et al., Seminars in Virology 7:61-73, 1996.

### Other Embodiments

All publications and patents cited in this specification are herein incorporated by reference as if each individual publication or patent were specifically and individually indicated to be incorporated by reference. Use of singular forms herein, such as “a” and “the,” does not exclude indication of the corresponding plural form, unless the context indicates to the contrary. Although the invention has been described in some detail by way of illustration and example for purposes of clarity of understanding, it will be readily apparent to those of ordinary skill in the art in light of the teachings of the invention that certain changes and modifications may be made thereto without departing from the spirit or scope of the appended claims.

Other embodiments are within the following claims.

1. A rhinovirus vector comprising an influenza virus HA₀ immunogen.

2. The rhinovirus vector of claim 1, wherein the rhinovirus vector is not pathogenic in humans.

3. The rhinovirus vector of claim 2, wherein the rhinovirus vector is Human Rhinovirus 14 (HRV14).

4. The rhinovirus vector of claim 1, wherein the rhinovirus vector further comprises an M2e peptide.

5. The rhinovirus vector of claim 1, wherein the influenza virus HA₀ immunogen is inserted at the site of a neutralizing immunogen selected from the group consisting of Neutralizing Immunogen I (NimI), Neutralizing Immunogen II (NimII), Neutralizing Immunogen III (NimIII), and Neutralizing Immunogen IV (NimIV), or at more than one of these sites.

6. The rhinovirus vector of claim 5, wherein the influenza virus HA₀ immunogen is inserted at the site of Neutralizing Immunogen II (NimII).

7. The rhinovirus vector of claim 6, wherein the influenza virus HA₀ immunogen is inserted between amino acids 158 and 160 of NimII.

8. The rhinovirus vector of claim 1, wherein the influenza virus HA₀ immunogen is flanked by linker sequences on one or both ends.

9. The rhinovirus vector of claim 1, wherein the rhinovirus vector is live.

10. The rhinovirus vector of claim 1, wherein the rhinovirus vector is inactivated.

11. A pharmaceutical composition comprising the rhinovirus vector of claim 1 and a pharmaceutically acceptable carrier or diluent.

12. The pharmaceutical composition of claim 11, further comprising an adjuvant.

13. The pharmaceutical composition of claim 11, further comprising one or more additional active ingredients.

14. The pharmaceutical composition of claim 11, further comprising a Hepatitis B core protein fused with M2e and/or HA₀ sequences.

15. The pharmaceutical composition of claim 11, comprising a rhinovirus vector comprising an HA₀ peptide and a rhinovirus vector comprising an M2e peptide.

16. A method of inducing an immune response to an influenza virus in a subject, the method comprising administering to the subject the pharmaceutical composition of claim 11.

17. The method of claim 16, wherein the subject does not have but is at risk of developing influenza virus infection.

18. The method of claim 16, wherein the subject has influenza virus infection.

19. The method of claim 16, wherein the composition is administered to the subject intranasally.

20. The method of claim 16, wherein the subject is a human.

21. A method of making a pharmaceutical composition, comprising admixing the rhinovirus vector of claim 1 and a pharmaceutically acceptable carrier or diluent.

22. A nucleic acid molecule encoding or corresponding to the genome of the rhinovirus vector of claim 1.

23. A NimII peptide comprising an inserted influenza virus HA_{O} immunogen.

24. A method of generating a rhinovirus vector comprising an influenza virus HA₀ immunogen, the method comprising the steps of:
(i) generating a library of recombinant rhinovirus vectors based on an infectious cDNA clone that comprises inserted influenza virus HA₀ immunogen sequences, and
(ii) selecting from the library recombinant viruses that (a) maintain inserted sequences upon passage, and (b) are neutralized with antibodies against the inserted sequence.

25. The method of claim 24, wherein the rhinovirus vector is human rhinovirus 14 (HRV14).

26. The method of claim 24, wherein the inserted influenza immunogen sequence is inserted at a position selected from the group consisting of NimI, NimII, NimIII, and NimIV.

27. The method of claim 24, further comprising insertion of an influenza virus M2e sequence.

28. The method of claim 24, wherein the inserted influenza virus HA₀ immunogen sequence is flanked on one or both ends with random linker sequences.

29. A method of cultivating a rhinovirus vector of claim 1, the method comprising passaging the vector in HeLa or MRC-5 cells.

30. (canceled)


SEQUENCE LISTING (87 sequences)

SEQ ID NO: 1 - protein, 25 aa, Artificial Sequence
  Human Influenza A Virus Subtype H1Nx
PSIQSRGLFGAIAGFIEGGWTGMVD

SEQ ID NO: 2 - protein, 25 aa, Artificial Sequence
  Human/Avian Influenza A Virus Subtype H2Nx
PQIESRGLFGAIAGFIEGGWQGMVD

SEQ ID NO: 3 - protein, 25 aa, Artificial Sequence
  Human Influenza A Virus Subtype H3Nx
PEKQTRGIFGAIAGFIENGWEGMVD

SEQ ID NO: 4 - protein, 25 aa, Artificial Sequence
  Human Influenza A Virus Subtype H5N1
RRRKKRGLFGAIAGFIEGGWQGMVD

SEQ ID NO: 5 - protein, 25 aa, Artificial Sequence
  Avian Influenza A Virus Subtype H6Nx
PQIETRGLFGAIAGFIEGGWTGMID

SEQ ID NO: 6 - protein, 25 aa, Artificial Sequence
  Avian/Equine/Human Influenza A Virus Subtype H7Nx
HKRKGRGLFGAIAGFIENGWEGLID

SEQ ID NO: 7 - protein, 25 aa, Artificial Sequence
  Avian/Human/Swine Influenza A Virus Subtype H9Nx
PARSSRGLFGAIAGFIEGGWSGLVA

SEQ ID NO: 8 - protein, 25 aa, Artificial Sequence
  Human Influenza B Virus
KLLKERGFFGAIAGFLEGGWEGMIA

SEQ ID NO: 9 - protein, 24 aa, Artificial Sequence
  Human Influenza A Virus Subtype H1N1
MSLLTEVETPIRNEWGCRCNDSSD

SEQ ID NO: 10 - protein, 24 aa, Artificial Sequence
  Human Influenza A Virus Subtype H5N1
MSLLTEVETPTRNEWECRCSDSSD

SEQ ID NO: 11 - protein, 24 aa, Artificial Sequence
  Human Influenza A Virus Subtype H5N1
MSLLTEVETLTRNGWGCRCSDSSD

SEQ ID NO: 12 - protein, 24 aa, Artificial Sequence
  Avian Influenza A Virus Subtype H5N1
MSLLTEVETLTRNGWGCRCSDSSD

SEQ ID NO: 13 - protein, 8 aa, Artificial Sequence
  Avian Influenza A Virus
EVETPTRN

SEQ ID NO: 14 - protein, 8 aa, Artificial Sequence
  Avian Influenza A Virus
EVETLTRN

SEQ ID NO: 15 - protein, 24 aa, Artificial Sequence
  Avian/Equine Influenza A Virus Subtype H7N7
MSLLTEVETPTRNGWECRCSDSSD

SEQ ID NO: 16 - protein, 24 aa, Artificial Sequence
  Avian Influenza A Virus Subtype H9Nx
MSLLTEVETPTRNGWECKCSDSSD

SEQ ID NO: 17 - protein, 24 aa, Artificial Sequence
  Avian Influenza A Virus Subtype H9Nx
MSLLTEVETHTRNGWGCRCSDSSD

SEQ ID NO: 18 - protein, 24 aa, Artificial Sequence
  Human Influenza A Virus Subtype H9N2
MSLLTEVETLTRNGWECKCSDSSD

SEQ ID NO: 19 - protein, 29 aa, Artificial Sequence
  Synthetic Sequence
SSASLLTEVETPIRNEWGCRCNDSSDNEV

SEQ ID NO: 20 - protein, 32 aa, Artificial Sequence
  Synthetic Construct
  MISC_FEATURE (4)..(6): Where Xaa is any amino acid
SSAXXXSLLTEVETPIRNEWGCRCNDSSDNEV

SEQ ID NO: 21 - protein, 26 aa, Artificial Sequence
  Synthetic Construct
  MISC_FEATURE (4)..(6): Where Xaa is any amino acid
SSAXXXSLLTEVETPIRNEWGCRNEV

SEQ ID NO: 22 - protein, 31 aa, Artificial Sequence
  Synthetic Construct
SSAQPASLLTEVETPIRNEWGSRNEVGGPVK

SEQ ID NO: 23 - protein, 30 aa, Artificial Sequence
  Synthetic construct
SSANVPEKQTQGIFGAIAGFIENEVGGPVK

SEQ ID NO: 24 - protein, 41 aa, Artificial Sequence
  Synthetic Construct
SSAQPASLLTEVETPIRNEWGSERGIFGAIAGFIEPGGPVK

SEQ ID NO: 25 - protein, 5 aa, Human Rhinovirus 14
SANEV

SEQ ID NO: 26 - protein, 7 aa, Human Rhinovirus 14
NANRQNE

SEQ ID NO: 27 - protein, 5 aa, Human Rhinovirus 14
DNHRE

SEQ ID NO: 28 - protein, 16 aa, Human Rhinovirus 14
NTEPVIKKRKGDIKSY

SEQ ID NO: 29 - DNA, 7212 bp, Human Rhinovirus 14
ttaaaacagcggatgggtatcccaccattcgacccattgggtgtagtactctggtactatgtacctttgtacgcctgtttctccccaaccacccttccttaaaattcccacccatgaaacgttagaagcttgacattaaagtacaataggtggcgccatatccaatggtgtctatgtacaagcacttctgtttccccggagcgaggtataggctgtacccactgccaaaagcctttaaccgttatccgccaaccaactacgtaacagttagtaccatcttgttcttgactggacgttcgatcaggtggattttccctccactagtttggtcgatgaggctaggaattccccacgggtgaccgtgtcctagcctgcgtggcggccaacccagcttatgctgggacgcccttttaaggacatggtgtgaagactcgcatgtgcttggttgtgagtcctccggcccctgaatgcggctaaccttaaccctggagccttatgccacgatccagtggttgtaaggtcgtaatgagcaactccgggacgggaccgactactttgggtgtccgtgtttctcatttttcttcatattgtcttatggtcacagcatatatatacatatactgtgatcatgggcgctcaggtttctacacagaaaagtggatctcacgaaaatcaaaacattttgaccaatggatcaaatcagactttcacagttataaattactataaggatgcagcaagtacatcatcagctggtcaatcactgtcaatggacccatctaagtttacagaaccagttaaagatctcatgcttaagggtgcaccagcattgaattcacccaatgttgaggcctgtggttatagtgatagagtacaacaaatcacactcgggaattcaacaataacaacacaagaagcagccaacgctgttgtgtgttatgctgaatggccagagtaccttccagatgtggacgctagtgatgtcaataaaacttcaaaaccagacacttctgtctgtaggttttacacattggatagtaagacatggacaacaggttctaaaggctggtgctggaaattaccagatgcactcaaagatatgggtgtgttcgggcaaaacatgtttttccactcactaggaagatcaggttacacagtacacgttcagtgcaatgccacaaaattccatagcggttgtctacttgtagttgtaataccagaacaccaactggcttcacatgagggtggcaatgtttcagttaaatacacattcacgcatccaggtgaacgtggtatagatttatcatctgcaaatgaagtgggagggcctgtcaaggatgtcatatacaatatgaatggtactttattaggaaatctgctcattttccctcaccagttcattaatctaagaaccaataatacagccacaatagtgataccatacataaactcagtacccattgattcaatgacacgtcacaacaatgtctcactgatggtcatccctattgcccctcttacagtaccaactggagcaactccctcactccctataacagtcacaatagcacctatgtgcactgagttctctgggataaggtccaagtcaattgtgccacaaggtttgccaactacaactttgccggggtcaggacaattcttgaccacagatgacaggcaatcccccagtgcactgccaaattatgagccaactccaagaatacacataccagggaaagttcataacttgctagaaattatacaggtagatacactcattcctatgaacaacacgcatacaaaagatgaggttaacagttacctcataccactaaatgcaaacaggcaaaatgagcaggtttttgggacaaacctgtttattggtgatggggtcttcaaaactactcttctgggtgaaattgttcagtactatacacattggtctggatcacttagattctctttgatgtatactggtcctgccttgtccagtgctaaactcattctagcatacaccccgcctggtgctcgtggtccacaggacaggagagaagcaatgctaggtactcatgttgtctgggatattggtctgcaatccaccatagtaatgacaataccatggacatcaggggtgcagtttagatatactgatccagatacatacaccagtgctggctttctatcatgttggtatcaaacttctcttatacttcccccagaaacgaccggccaggtctacttattatcattcataagtgcatgtccagattttaagcttaggctgatgaaagatactcaaactatctcacagactgttgcactcactgaaggcttaggtgatgaattagaagaagtcatcgttgagaaaacgaaacagacggtggcctcaatctcatctggtccaaaacacacacaaaaagtccccatactaactgcaaacgaaacaggggccacaatgcctgttcttccatcagacagcatagaaaccagaactacctacatgcactttaatggttcagaaactgatgtagaatgctttttgggtcgtgcagcttgtgtgcatgtaactgaaatacaaaacaaagatgctactggaatagataatcacagagaagcaaaattgttcaatgattggaaaatcaacctgtccagccttgtccaacttagaaagaaactagaactcttcacttatgttaggtttgattctgagtataccatactggccactgcatctcaacctgattcagcaaactattcaagcaatttggtggtccaagccatgtatgttccacctggtgccccgaatccaaaagagtgggacgattacacatggcaaagtgcttcaaaccccagtgtattcttcaaggtgggggatacatccaggtttagtgtgccttatgtaggattggcatcagcatataattgtttttatgatggttactcacatgatgatgcagaaactcagtatggcataactgttctaaaccatatgggtagtatggcattcagaatagtaaatgaacatgatgaacataaaactcttgtcaagatcagagtttatcacagggcaaagcacgttgaagcatggattccaagagcacccagagcactaccctacacatcaatagggcgcacaaattatcctaagaatacagaaccagtaattaagaagaggaaaggtgacattaaatcctatggtttaggacctaggtacggtgggatttatacatcaaatgttaaaataatgaattaccacttgatgacaccagaagaccaccataatctgatagcaccctatccaaatagagatttagcaatagtctcaacaggaggacatggtgcagaaacaataccacactgtaactgtacatcaggtgtttactattccacatattacagaaagtattaccccataatttgtgaaaagcccaccaacatctggattgaaggaaacccttattacccaagtaggtttcaagcaggagtgatgaaaggggttgggccagcagaaccaggagactgcggtgggattttgagatgcatacatggtcccattggattgttaacagctggaggtagtggatatgtttgttttgctgacatacgacagttggagtgtatcgcagaggaacaggggctgagtgattacatcacaggtttgggtagagcttttggtgtcgggttcactgaccaaatctcaacaaaagtcacagaactacaagaagtggcgaaagatttcctcaccacaaaagttttgtccaaagtggtcaaaatggtttcagctttagtgatcatttgcagaaatcatgatgacttggtcactgttacggccactctagcactacttggatgtgatggatctccctggagatttctgaagatgtacatttccaaacactttcaggtgccttacattgaaagacaagcaaatgatggatggttcagaaagtttaatgatgcatgtaatgctgcaaagggattggaatggattgctaataagatttccaaactgattgaatggataaaaaacaaagtacttccccaagccaaagaaaaactagaattttgtagtaaactcaaacaacttgatatactagagagacaaataaccaccatgcatatctcgaatccaacacaggaaaaacgagagcagttgttcaacaacgtattgtggttggaacaaatgtcgcaaaagtttgccccacattatgccgttgaatcaaaaagaatcagggaactcaagaacaaaatggtaaattatatgcaatttaaaagtaaacaaagaactgaaccagtgtgtgtattaatccatggtacacccggttctggtaaatcattaacaacatccattgtgggacgtgcaattgcagaacacttcaattcagcagtatattcacttccaccagatcccaagcactttgatggttatcagcaacaggaagttgtgattatggatgatctgaaccaaaatccagatggacaggatataagcatgttttgtcaaatggtttcttcagtggatttcttgcctccaatggctagtttagataacaagggcatgttattcaccagtaattttgttctagcctccacaaattctaacacactaagccccccaacaatcttgaatcctgaagctttagtcaggagatttggttttgacctggatatatgtttgcatactacctacacaaagaatggaaaactcaatgcaggcatgtcaaccaagacatgcaaagattgccatcaaccatctaatttcaagaaatgttgccccctggtctgtggaaaagctattagcttggtagacagaactaccaacgttaggtatagtgtggatcaactggtcacagctattataagtgatttcaagagcaaaatgcaaattacagattccctagaaacactgtttcaaggaccagtgtataaagatttagagattgatgtttgcaacacaccacctccagaatgtatcaacgatttactgaaatctgtagattcagaagagattagggaatattgtaagaagaagaaatggattatacctgaaattcctaccaacatagaaagggctatgaatcaagccagcatgattattaatactattctgatgtttgtcagtacattaggtattgtttatgtcatttataaattgtttgctcaaactcaaggaccatattctggtaacccgcctcacaataaactaaaagccccaactttacgcccagttgttgtgcaaggaccaaacacagaatttgcactatccctgttaaggaaaaacataatgactataacaacctcaaagggagagttcacagggttaggcatacatgatcgtgtctgtgtgatacccacacacgcacagcctggtgatgatgtactagtgaatggtcagaaaattagagttaaggataagtacaaattagtagatccagagaacattaatctagagcttacagtgttgactttagatagaaatgaaaaattcagagatatcaggggatttatatcagaagatctagaaggtgtggatgccactttggtagtacattcaaataactttaccaacactatcttagaagttggccctgtaacaatggcaggacttattaatttgagtagcacccccactaacagaatgattcgttatgattatgcaacaaaaactgggcagtgtggaggtgtgctgtgtgctactggtaagatctttggtattcatgttggcggtaatggaagacaaggattttcagctcaacttaaaaaacaatattttgtagagaaacaaggccaagtaatagctagacataaggttagggagtttaacataaatccagtcaacacgccaaccaagtcaaaattacatcccagtgtattctatgatgttttcccaggtgacaaggaacctgctgtattgagtgacaatgatcccagactggaagttaaattgactgaatcattattctctaagtacaaggggaatgtaaatacggaacccactgaaaatatgcttgtggctgtagaccattatgcagggcaactattatcactagatatccccacttctgaacttacactaaaagaagcattatatggagtagatggactagaacctatagatattacaaccagtgcaggatttccctatgtgagtcttgggatcaaaaagagagacattctgaacaaagagacccaggacacagaaaagatgaagttttatctagacaagtatggcattgacttgcctctagttacatatattaaggatgaattaagaagtgttgacaaagtccgattagggaaaagtagattaattgaagcctccagtttgaatgattctgttaacatgagaatgaaactaggcaacctttacaaagcattccatcaaaatcccggtgttctgactgggtcagcagtgggttgtgatcctgatgtgttttggtctgtcatcccttgcttaatggatgggcacctgatggcatttgattactctaattttgatgcctctttgtcaccagtttggtttgtctgtctagagaaggttttgaccaagttaggctttgcaggctcttcattaattcaatcaatttgtaatacccatcatatctttagggatgaaatatatgtggttgaaggtggcatgccctcagggtgttcaggaaccagcatattcaattccatgatcaacaacataatcattaggactttgatattagatgcatataaaggaatagatttagacaaacttaaaatcttagcttacggtgatgatttgattgtttcttatccttatgaactggatccacaagtgttggcaactcttggtaaaaattatggactaaccatcacacccccagacaaatctgaaacttttacaaaaatgacatgggaaaacttgacatttttaaagagatacttcaagcctgatcaacaatttccctttttggttcacccagttatgcccatgaaagatatacatgagtcaatcagatggacaaaggatcctaaaaacacacaggatcacgtccgatcattatgcatgttagcatggcactcaggagaaaaagagtacaatgaattcattcagaagatcagaactactgacattggaaaatgtctaattctcccagaatacagcgtacttaggaggcgctggttggacctcttttaggttaacaatatagacacttaatttgagtagaagtaggagtttat

SEQ ID NO: 30 - protein, 2179 aa, Human Rhinovirus 14
MGAQVSTQKSGSHENQNILTNGSNQTFTVINYYKDAASTSSAGQSLSMDPSKFTEPVKDLMLKGAPALNSPNVEACGYSDRVQQITLGNSTITTQEAANAVVCYAEWPEYLPDVDASDVNKTSKPDTSVCRFYTLDSKTWTTGSKGWCWKLPDALKDMGVFGQNMFFHSLGRSGYTVHVQCNATKFHSGCLLVVVIPEHQLASHEGGNVSVKYTFTHPGERGIDLSSANEVGGPVKDVIYNMNGTLLGNLLIFPHQFINLRTNNTATIVIPYINSVPIDSMTRHNNVSLMVIPIAPLTVPTGATPSLPITVTIAPMCTEFSGIRSKSIVPQGLPTTTLPGSGQFLTTDDRQSPSALPNYEPTPRIHIPGKVHNLLEIIQVDTLIPMNNTHTKDEVNSYLIPLNANRQNEQVFGTNLFIGDGVFKTTLLGEIVQYYTHWSGSLRFSLMYTGPALSSAKLILAYTPPGARGPQDRREAMLGTHVVWDIGLQSTIVMTIPWTSGVQFRYTDPDTYTSAGFLSCWYQTSLILPPETTGQVYLLSFISACPDFKLRLMKDTQTISQTVALTEGLGDELEEVIVEKTKQTVASISSGPKHTQKVPILTANETGATMPVLPSDSIETRTTYMHFNGSETDVECFLGRAACVHVTEIQNKDATGIDNHREAKLFNDWKINLSSLVQLRKKLELFTYVRFDSEYTILATASQPDSANYSSNLVVQAMYVPPGAPNPKEWDDYTWQSASNPSVFFKVGDTSRFSVPYVGLASAYNCFYDGYSHDDAETQYGITVLNHMGSMAFRIVNEHDEHKTLVKIRVYHRAKHVEAWIPRAPRALPYTSIGRTNYPKNTEPVIKKRKGDIKSYGLGPRYGGIYTSNVKIMNYHLMTPEDHHNLIAPYPNRDLAIVSTGGHGAETIPHCNCTSGVYYSTYYRKYYPIICEKPTNIWIEGNPYYPSRFQAGVMKGVGPAEPGDCGGILRCIHGPIGLLTAGGSGYVCFADIRQLECIAEEQGLSDYITGLGRAFGVGFTDQISTKVTELQEVAKDFLTTKVLSKVVKMVSALVIICRNHDDLVTVTATLALLGCDGSPWRFLKMYISKHFQVPYIERQANDGWFRKFNDACNAAKGLEWIANKISKLIEWIKNKVLPQAKEKLEFCSKLKQLDILERQITTMHISNPTQEKREQLFNNVLWLEQMSQKFAPHYAVESKRIRELKNKMVNYMQFKSKQRTEPVCVLIHGTPGSGKSLTTSIVGRAIAEHFNSAVYSLPPDPKHFDGYQQQEVVIMDDLNQNPDGQDISMFCQMVSSVDFLPPMASLDNKGMLFTSNFVLASTNSNTLSPPTILNPEALVRRFGFDLDICLHTTYTKNGKLNAGMSTKTCKDCHQPSNFKKCCPLVCGKAISLVDRTTNVRYSVDQLVTAIISDFKSKMQITDSLETLFQGPVYKDLEIDVCNTPPPECINDLLKSVDSEEIREYCKKKKWIIPEIPTNIERAMNQASMIINTILMFVSTLGIVYVIYKLFAQTQGPYSGNPPHNKLKAPTLRPVVVQGPNTEFALSLLRKNIMTITTSKGEFTGLGIHDRVCVIPTHAQPGDDVLVNGQKIRVKDKYKLVDPENINLELTVLTLDRNEKFRDIRGFISEDLEGVDATLVVHSNNFTNTILEVGPVTMAGLINLSSTPTNRMIRYDYATKTGQCGGVLCATGKIFGIHVGGNGRQGFSAQLKKQYFVEKQGQVIARHKVREFNINPVNTPTKSKLHPSVFYDVFPGDKEPAVLSDNDPRLEVKLTESLFSKYKGNVNTEPTENMLVAVDHYAGQLLSLDIPTSELTLKEALYGVDGLEPIDITTSAGFPYVSLGIKKRDILNKETQDTEKMKFYLDKYGIDLPLVTYIKDELRSVDKVRLGKSRLIEASSLNDSVNMRMKLGNLYKAFHQNPGVLTGSAVGCDPDVFWSVIPCLMDGHLMAFDYSNFDASLSPVWFVCLEKVLTKLGFAGSSLIQSICNTHHIFRDEIYVVEGGMPSGCSGTSIFNSMINNIIIRTLILDAYKGIDLDKLKILAYGDDLIVSYPYELDPQVLATLGKNYGLTITPPDKSETFTKMTWENLTFLKRYFKPDQQFPFLVHPVMPMKDIHESIRWTKDPKNTQDHVRSLCMLAWHSGEKEYNEFIQKIRTTDIGKCLILPEYSVLRRRWLDLF

SEQ ID NO: 31 - protein, 19 aa, Artificial Sequence
  Human Influenza A Virus Subtype H1Nx
NIPSIQSRGLFGAIAGFIE

SEQ ID NO: 32 - protein, 19 aa, Artificial Sequence
  Human Influenza A Virus Subtype H3Nx
NVPEKQTRGIFGAIAGFIE

SEQ ID NO: 33 - protein, 19 aa, Human Influenza B Virus
PAKLLKERGFFGAIAGFLE

SEQ ID NO: 34 - protein, 11 aa, Human Influenza A Virus Subtype H3Nx
RGIFGAIAGFI

SEQ ID NO: 35 - protein, 18 aa, Human Influenza A Virus Subtype H3Nx
NVPEKQTQGIFGAIAGFI

SEQ ID NO: 36 - protein, 23 aa, Human Influenza A Virus Subtype H1N1
SLLTEVETPIRNEWGCRCNDSSD

SEQ ID NO: 37 - protein, 17 aa, Human Influenza A Virus Subtype H1N1
SLLTEVETPIRNEWGCR

SEQ ID NO: 38 - protein, 6 aa, Human Influenza B Virus
MLEPFQ

SEQ ID NO: 39 - protein, 29 aa, Artificial Sequence
  Synthetic Construct
SLLTEVETPIRNEWGSERGIFGAIAGFIE

SEQ ID NO: 40 - protein, 20 aa, Human Influenza B Virus
MNNATFNYTNVNPISHIRGS

SEQ ID NO: 41 - protein, 20 aa, Human Papillomavirus 16
QLYKTCKQAGTCPPDIIPKV

SEQ ID NO: 42 - protein, 26 aa, Artificial Sequence
  Synthetic Construct
GHTSLLKEVETPIRNEWGSRSNDSSD

SEQ ID NO: 43 - protein, 20 aa, Artificial Sequence
  Synthetic construct
QPASLLTEVETPIRNEWGSR

SEQ ID NO: 44 - protein, 19 aa, Artificial Sequence
  Synthetic construct
NVPEKQTQGIFGAIAGFIE

SEQ ID NO: 45 - protein, 8 aa, Hepatitis C Virus
STNPKPQR

SEQ ID NO: 46 - protein, 10 aa, Hepatitis C Virus
YLLPRRGPRL

SEQ ID NO: 47 - protein, 9 aa, Hepatitis C Virus
GPRLGVRAT

SEQ ID NO: 48 - protein, 20 aa, Hepatitis C Virus
YPWPLYGNEGCGWAGWLLSP

SEQ ID NO: 49 - protein, 16 aa, Hepatitis C Virus
GFADLMGYIPLVGAPL

SEQ ID NO: 50 - protein, 9 aa, Hepatitis C Virus
DLMGYIPLV

SEQ ID NO: 51 - protein, 10 aa, Hepatitis C Virus
LLALLSCLTV

SEQ ID NO: 52 - protein, 20 aa, Hepatitis C Virus
REGNASRCWVAVTPTVATRD

SEQ ID NO: 53 - protein, 9 aa, Hepatitis C Virus
STGLIHLHQ

SEQ ID NO: 54 - protein, 10 aa, Hepatitis C Virus
LLADARVCSC

SEQ ID NO: 55 - protein, 11 aa, Hepatitis C Virus
CWHYPPRPCGI

SEQ ID NO: 56 - protein, 10 aa, Hepatitis C Virus
CVIGGVGNNT

SEQ ID NO: 57 - protein, 10 aa, Hepatitis C Virus
RRLTDFAQGW

SEQ ID NO: 58 - protein, 8 aa, Hepatitis C Virus
TINYTIFK

SEQ ID NO: 59 - protein, 27 aa, Hepatitis C Virus
ETHVTGGNAGRTTAGLVGLLTPGAKQN

SEQ ID NO: 60 - protein, 27 aa, Hepatitis C Virus
IQLINTNGSWHINSTALNCNESLNTGW

SEQ ID NO: 61 - protein, 20 aa, Hepatitis C Virus
LFYQHKFNSSGCPERLASCR

SEQ ID NO: 62 - protein, 36 aa, Hepatitis C Virus
PSPVVVGTTDRSGAPTYSWGANDTDVFVLNNTRPPL

SEQ ID NO: 63 - protein, 6 aa, Hepatitis C Virus
IQLINT

SEQ ID NO: 64 - protein, 7 aa, Human Papillomavirus 16
DRAHYNI

SEQ ID NO: 65 - protein, 9 aa, Human Papillomavirus 16
RAHYNIVTF

SEQ ID NO: 66 - protein, 5 aa, Human Papillomavirus 16
EYMLD

SEQ ID NO: 67 - protein, 4 aa, Human Papillomavirus 16
IDGP

SEQ ID NO: 68 - protein, 5 aa, Human Papillomavirus 16
QAEPD

SEQ ID NO: 69 - protein, 11 aa, Human Papillomavirus 18
VNHQHLPARRA

SEQ ID NO: 70 - protein, 10 aa, Human Papillomavirus 18
DDLRAFQQLF

SEQ ID NO: 71 - protein, 11 aa, Artificial Sequence
  Synthetic Construct
GIFGAIAGFIE

SEQ ID NO: 72 - protein, 23 aa, Human Influenza A Virus Subtype H1N1
SLLTEVETPIRNEWECRCNGSSD

SEQ ID NO: 73 - protein, 23 aa, Human Influenza A Virus Subtype H1N1
SLLTEVETPTKNEWGCRCNDSSD

SEQ ID NO: 74 - protein, 23 aa, Human Influenza A Virus Subtype H3N2
SLLTEVETPIRNEWGCRCNGSSD

SEQ ID NO: 75 - protein, 23 aa, Human Influenza A Virus Subtype H1N1
SLLTEVETPIRNEWECRCNDSSD

SEQ ID NO: 76 - skipped

SEQ ID NO: 77 - DNA, 60 bp, Artificial Sequence
  Synthetic Construct
gggaatgtaccagagaaacaaactcaaggtatatttggtgcaatagcaggttttatagaa

SEQ ID NO: 78 - DNA, 11500 bp, Artificial Sequence
  Synthetic Construct
gggttaaaacagcggatgggtatcccaccattcgacccattgggtgtagtactctggtactatgtacctttgtacgcctgtttctccccaaccacccttccttaaaattcccacccatgaaacgttagaagcttgacattaaagtacaataggtggcgccatatccaatggtgtctatgtacaagcacttctgtttccccggagcgaggtataggctgtacccactgccaaaagcctttaaccgttatccgccaaccaactacgtaacagttagtaccatcttgttcttgactggacgttcgatcaggtggattttccctccactagtttggtcgatgaggctaggaattccccacgggtgaccgtgtcctagcctgcgtggcggccaacccagcttatgctgggacgcccttttaaggacatggtgtgaagactcgcatgtgcttggttgtgagtcctccggcccctgaatgcggctaaccttaaccctggagccttatgccacgatccagtggttgtaaggtcgtaatgagcaactccgggacgggaccgactactttgggtgtccgtgtttctcatttttcttcatattgtcttatggtcacagcatatatatacatatactgtgatcatgggcgctcaggtttctacacagaaaagtggatctcacgaaaatcaaaacattttgaccaatggatcaaatcagactttcacagttataaattactataaggatgcagcaagtacatcatcagctggtcaatcactgtcaatggacccatctaagtttacagaaccagttaaagatctcatgcttaagggtgcaccagcattgaattcacccaatgttgaggcctgtggttatagtgatagagtacaacaaatcacactcgggaattcaacaataacaacacaagaagcagccaacgctgttgtgtgttatgctgaatggccagagtaccttccagatgtggacgctagtgatgtcaataaaacttcaaaaccagacacttctgtctgtaggttttacacattggatagtaagacatggacaacaggttctaaaggctggtgctggaaattaccagatgcactcaaagatatgggtgtgttcgggcaaaacatgtttttccactcactaggaagatcaggttacacagtacacgttcagtgcaatgccacaaaattccatagcggttgtctacttgtagttgtaataccagaacaccaactggcttcacatgagggtggcaatgtttcagttaaatacacattcacgcatccaggtgaacgtggtatagatttatcatctgcagggaatgtaccagagaaacaaactcaaggtatatttggtgcaatagcaggttttatagaaaatgaagtgggagggcctgtcaaggatgtcatatacaatatgaatggtactttattaggaaatctgctcattttccctcaccagttcattaatctaagaaccaataatacagccacaatagtgataccatacataaactcagtacccattgattcaatgacacgtcacaacaatgtctcactgatggtcatccctattgcccctcttacagtaccaactggagcaactccctcactccctataacagtcacaatagcacctatgtgcactgagttctctgggataaggtccaagtcaattgtgccacaaggtttgccaactacaactttgccggggtcaggacaattcttgaccacagatgacaggcaatcccccagtgcactgccaaattatgagccaactccaagaatacacataccagggaaagttcataacttgctagaaattatacaggtagatacactcattcctatgaacaacacgcatacaaaagatgaggttaacagttacctcataccactaaatgcaaacaggcaaaatgagcaggtttttgggacaaacctgtttattggtgatggggtcttcaaaactactcttctgggtgaaattgttcagtactatacacattggtctggatcacttagattctctttgatgtatactggtcctgccttgtccagtgctaaactcattctagcatacaccccgcctggtgctcgtggtccacaggacaggagagaagcaatgctaggtactcatgttgtctgggatattggtctgcaatccaccatagtaatgacaataccatggacatcaggggtgcagtttagatatactgatccagatacatacaccagtgctggctttctatcatgttggtatcaaacttctcttatacttcccccagaaacgaccggccaggtctacttattatcattcataagtgcatgtccagattttaagcttaggctgatgaaagatactcaaactatctcacagactgttgcactcactgaaggcttaggtgatgaattagaagaagtcatcgttgagaaaacgaaacagacggtggcctcaatctcatctggtccaaaacacacacaaaaagtccccatactaactgcaaacgaaacaggggccacaatgcctgttcttccatcagacagcatagaaaccagaactacctacatgcactttaatggttcagaaactgatgtagaatgctttttgggtcgtgcagcttgtgtgcatgtaactgaaatacaaaacaaagatgctactggaatagataatcacagagaagcaaaattgttcaatgattggaaaatcaacctgtccagccttgtccaacttagaaagaaactagaactcttcacttatgttaggtttgattctgagtataccatactggccactgcatctcaacctgattcagcaaactattcaagcaatttggtggtccaagccatgtatgttccacctggtgccccgaatccaaaagagtgggacgattacacatggcaaagtgcttcaaaccccagtgtattcttcaaggtgggggatacatccaggtttagtgtgccttatgtaggattggcatcagcatataattgtttttatgatggttactcacatgatgatgcagaaactcagtatggcataactgttctaaaccatatgggtagtatggcattcagaatagtaaatgaacatgatgaacataaaactcttgtcaagatcagagtttatcacagggcaaagcacgttgaagcatggattccaagagcacccagagcactaccctacacatcaatagggcgcacaaattatcctaagaatacagaaccagtaattaagaagaggaaaggtgacattaaatcctatggtttaggacctaggtacggtgggatttatacatcaaatgttaaaataatgaattaccacttgatgacaccagaagaccaccataatctgatagcaccctatccaaatagagatttagcaatagtctcaacaggaggacatggtgcagaaacaataccacactgtaactgtacatcaggtgtttactattccacatattacagaaagtattaccccataatttgtgaaaagcccaccaacatctggattgaaggaaacccttattacccaagtaggtttcaagcaggagtgatgaaaggggttgggccagcagaaccaggagactgcggtgggattttgagatgcatacatggtcccattggattgttaacagctggaggtagtggatatgtttgttttgctgacatacgacagttggagtgtatcgcagaggaacaggggctgagtgattacatcacaggtttgggtagagcttttggtgtcgggttcactgaccaaatctcaacaaaagtcacagaactacaagaagtggcgaaagatttcctcaccacaaaagttttgtccaaagtggtcaaaatggtttcagctttagtgatcatttgcagaaatcatgatgacttggtcactgttacggccactctagcactacttggatgtgatggatctccctggagatttctgaagatgtacatttccaaacactttcaggtgccttacattgaaagacaagcaaatgatggatggttcagaaagtttaatgatgcatgtaatgctgcaaagggattggaatggattgctaataagatttccaaactgattgaatggataaaaaacaaagtacttccccaagccaaagaaaaactagaattttgtagtaaactcaaacaacttgatatactagagagacaaataaccaccatgcatatctcgaatccaacacaggaaaaacgagagcagttgttcaacaacgtattgtggttggaacaaatgtcgcaaaagtttgccccacattatgccgttgaatcaaaaagaatcagggaactcaagaacaaaatggtaaattatatgcaatttaaaagtaaacaaagaactgaaccagtgtgtgtattaatccatggtacacccggttctggtaaatcattaacaacatccattgtgggacgtgcaattgcagaacacttcaattcagcagtatattcacttccaccagatcccaagcactttgatggttatcagcaacaggaagttgtgattatggatgatctgaaccaaaatccagatggacaggatataagcatgttttgtcaaatggtttcttcagtggatttcttgcctccaatggctagtttagataacaagggcatgttattcaccagtaattttgttctagcctccacaaattctaacacactaagccccccaacaatcttgaatcctgaagctttagtcaggagatttggttttgacctggatatatgtttgcatactacctacacaaagaatggaaaactcaatgcaggcatgtcaaccaagacatgcaaagattgccatcaaccatctaatttcaagaaatgttgccccctggtctgtggaaaagctattagcttggtagacagaactaccaacgttaggtatagtgtggatcaactggtcacagctattataagtgatttcaagagcaaaatgcaaattacagattccctagaaacactgtttcaaggaccagtgtataaagatttagagattgatgtttgcaacacaccacctccagaatgtatcaacgatttactgaaatctgtagattcagaagagattagggaatattgtaagaagaagaaatggattatacctgaaattcctaccaacatagaaagggctatgaatcaagccagcatgattattaatactattctgatgtttgtcagtacattaggtattgtttatgtcatttataaattgtttgctcaaactcaaggaccatattctggtaacccgcctcacaataaactaaaagccccaactttacgcccagttgttgtgcaaggaccaaacacagaatttgcactatccctgttaaggaaaaacataatgactataacaacctcaaagggagagttcacagggttaggcatacatgatcgtgtctgtgtgatacccacacacgcacagcctggtgatgatgtactagtgaatggtcagaaaattagagttaaggataagtacaaattagtagatccagagaacattaatctagagcttacagtgttgactttagatagaaatgaaaaattcagagatatcaggggatttatatcagaagatctagaaggtgtggatgccactttggtagtacattcaaataactttaccaacactatcttagaagttggccctgtaacaatggcaggacttattaatttgagtagcacccccactaacagaatgattcgttatgattatgcaacaaaaactgggcagtgtggaggtgtgctgtgtgctactggtaagatctttggtattcatgttggcggtaatggaagacaaggattttcagctcaacttaaaaaacaatattttgtagagaaacaaggccaagtaatagctagacataaggttagggagtttaacataaatccagtcaacacgccaaccaagtcaaaattacatcccagtgtattctatgatgttttcccaggtgacaaggaacctgctgtattgagtgacaatgatcccagactggaagttaaattgactgaatcattattctctaagtacaaggggaatgtaaatacggaacccactgaaaatatgcttgtggctgtagaccattatgcagggcaactattatcactagatatccccacttctgaacttacactaaaagaagcattatatggagtagatggactagaacctatagatattacaaccagtgcaggatttccctatgtgagtcttgggatcaaaaagagagacattctgaacaaagagacccaggacacagaaaagatgaagttttatctagacaagtatggcattgacttgcctctagttacatatattaaggatgaattaagaagtgttgacaaagtccgattagggaaaagtagattaattgaagcctccagtttgaatgattctgttaacatgagaatgaaactaggcaacctttacaaagcattccatcaaaatcccggtgttctgactgggtcagcagtgggttgtgatcctgatgtgttttggtctgtcatcccttgcttaatggatgggcacctgatggcatttgattactctaattttgatgcctctttgtcaccagtttggtttgtctgtctagagaaggttttgaccaagttaggctttgcaggctcttcattaattcaatcaatttgtaatacccatcatatctttagggatgaaatatatgtggttgaaggtggcatgccctcagggtgttcaggaaccagcatattcaattccatgatcaacaacataatcattaggactttgatattagatgcatataaaggaatagatttagacaaacttaaaatcttagcttacggtgatgatttgattgtttcttatccttatgaactggatccacaagtgttggcaactcttggtaaaaattatggactaaccatcacacccccagacaaatctgaaacttttacaaaaatgacatgggaaaacttgacatttttaaagagatacttcaagcctgatcaacaatttccctttttggttcacccagttatgcccatgaaagatatacatgagtcaatcagatggacaaaggatcctaaaaacacacaggatcacgtccgatcattatgcatgttagcatggcactcaggagaaaaagagtacaatgaattcattcagaagatcagaactactgacattggaaaatgtctaattctcccagaatacagcgtacttaggaggcgctggttggacctcttttaggttaacaatatagacacttaatttgagtagaagtaggagtttataaaaaaaaaaaaaaaaaaaaaaaaaaaaaaaaaaaaaaaaaaacgcgtggggatcctctagagtcgacggtaccaggaggaactatatccggattggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcaggtggcacttttcggggaaatgtgcgcggaacccctatttgtttatttttctaaatacattcaaatatgtatccgctcatgaattaattcttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcataacaccccttgtattactgtttatgtaagcagacagttttattgttcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtcgagatcccggtgcctaatgagtgagctaacttacattaattgcgttctgcagcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctcctgcagctaatacgactcactata

SEQ ID NO: 79 - protein, 876 aa, Artificial Sequence
  Synthetic Construct
MGAQVSTQKSGSHENQNILTNGSNQTFTVINYYKDAASTSSAGQSLSMDPSKFTEPVKDLMLKGAPALNSPNVEACGYSDRVQQITLGNSTITTQEAANAVVCYAEWPEYLPDVDASDVNKTSKPDTSVCRFYTLDSKTWTTGSKGWCWKLPDALKDMGVFGQNMFFHSLGRSGYTVHVQCNATKFHSGCLLVVVIPEHQLASHEGGNVSVKYTFTHPGERGIDLSSAQPASLLTEVETPIRNEWGSRNEVGGPVKDVIYNMNGTLLGNLLIFPHQFINLRTNNTATIVIPYINSVPIDSMTRHNNVSLMVIPIAPLTVPTGATPSLPITVTIAPMCTEFSGIRSKSIVPQGLPTTTLPGSGQFLTTDDRQSPSALPNYEPTPRIHIPGKVHNLLEIIQVDTLIPMNNTHTKDEVNSYLIPLNANRQNEQVFGTNLFIGDGVFKTTLLGEIVQYYTHWSGSLRFSLMYTGPALSSAKLILAYTPPGARGPQDRREAMLGTHVVWDIGLQSTIVMTIPWTSGVQFRYTDPDTYTSAGFLSCWYQTSLILPPETTGQVYLLSFISACPDFKLRLMKDTQTISQTVALTEGLGDELEEVIVEKTKQTVASISSGPKHTQKVPILTANETGATMPVLPSDSIETRTTYMHFNGSETDVECFLGRAACVHVTEIQNKDATGIDNHREAKLFNDWKINLSSLVQLRKKLELFTYVRFDSEYTILATASQPDSANYSSNLVVQAMYVPPGAPNPKEWDDYTWQSASNPSVFFKVGDTSRFSVPYVGLASAYNCFYDGYSHDDAETQYGITVLNHMGSMAFRIVNEHDEHKTLVKIRVYHRAKHVEAWIPRAPRALPYTSIGRTNYPKNTEPVIKKRKGDIKSY

SEQ ID NO: 80 - DNA, 10903 bp, Artificial Sequence
  Synthetic construct
gggttaaaacagcggatgggtatcccaccattcgacccattgggtgtagtactctggtactatgtacctttgtacgcctgtttctccccaaccacccttccttaaaattcccacccatgaaacgttagaagcttgacattaaagtacaataggtggcgccatatccaatggtgtctatgtacaagcacttctgtttccccggagcgaggtataggctgtacccactgccaaaagcctttaaccgttatccgccaaccaactacgtaacagttagtaccatcttgttcttgactggacgttcgatcaggtggattttccctccactagtttggtcgatgaggctaggaattccccacgggtgaccgtgtcctagcctgcgtggcggccaacccagcttatgctgggacgcccttttaaggacatggtgtgaagactcgcatgtgcttggttgtgagtcctccggcccctgaatgcggctaaccttaaccctggagccttatgccacgatccagtggttgtaaggtcgtaatgagcaactccgggacgggaccgactactttgggtgtccgtgtttctcatttttcttcatattgtcttatggtcacagcatatatatacatatactgtgatcatgggcgctcaggtttctacacagaaaagtggatctcacgaaaatcaaaacattttgaccaatggatcaaatcagactttcacagttataaattactataaggatgcagcaagtacatcatcagctggtcaatcactgtcaatggacccatctaagtttacagaaccagttaaagatctcatgcttaagggtgcaccagcattgaattcacccaatgttgaggcctgtggttatagtgatagagtacaacaaatcacactcgggaattcaacaataacaacacaagaagcagccaacgctgttgtgtgttatgctgaatggccagagtaccttccagatgtggacgctagtgatgtcaataaaacttcaaaaccagacacttctgtctgtaggttttacacattggatagtaagacatggacaacaggttctaaaggctggtgctggaaattaccagatgcactcaaagatatgggtgtgttcgggcaaaacatgtttttccactcactaggaagatcaggttacacagtacacgttcagtgcaatgccacaaaattccatagcggttgtctacttgtagttgtaataccagaacaccaactggcttcacatgagggtggcaatgtttcagttaaatacacattcacgcatccaggtgaacgtggtatagatttatcatctgcacagcccgcatcattattaacagaagttgaaacaccaataagaaatgaatggggctcgagaaatgaagtgggagggcctgtcaaggatgtcatatacaatatgaatggtactttattaggaaatctgctcattttccctcaccagttcattaatctaagaaccaataatacagccacaatagtgataccatacataaactcagtacccattgattcaatgacacgtcacaacaatgtctcactgatggtcatccctattgcccctcttacagtaccaactggagcaactccctcactccctataacagtcacaatagcacctatgtgcactgagttctctgggataaggtccaagtcaattgtgccacaaggtttgccaactacaactttgccggggtcaggacaattcttgaccacagatgacaggcaatcccccagtgcactgccaaattatgagccaactccaagaatacacataccagggaaagttcataacttgctagaaattatacaggtagatacactcattcctatgaacaacacgcatacaaaagatgaggttaacagttacctcataccactaaatgcaaacaggcaaaatgagcaggtttttgggacaaacctgtttattggtgatggggtcttcaaaactactcttctgggtgaaattgttcagtactatacacattggtctggatcacttagattctctttgatgtatactggtcctgccttgtccagtgctaaactcattctagcatacaccccgcctggtgctcgtggtccacaggacaggagagaagcaatgctaggtactcatgttgtctgggatattggtctgcaatccaccatagtaatgacaataccatggacatcaggggtgcagtttagatatactgatccagatacatacaccagtgctggctttctatcatgttggtatcaaacttctcttatacttcccccagaaacgaccggccaggtctacttattatcattcataagtgcatgtccagattttaagcttaggctgatgaaagatactcaaactatctcacagactgttgcactcactgaaggcttaggtgatgaattagaagaagtcatcgttgagaaaacgaaacagacggtggcctcaatctcatctggtccaaaacacacacaaaaagtccccatactaactgcaaacgaaacaggggccacaatgcctgttcttccatcagacagcatagaaaccagaactacctacatgcactttaatggttcagaaactgatgtagaatgctttttgggtcgtgcagcttgtgtgcatgtaactgaaatacaaaacaaagatgctactggaatagataatcacagagaagcaaaattgttcaatgattggaaaatcaacctgtccagccttgtccaacttagaaagaaactagaactcttcacttatgttaggtttgattctgagtataccatactggccactgcatctcaacctgattcagcaaactattcaagcaatttggtggtccaagccatgtatgttccacctggtgccccgaatccaaaagagtgggacgattacacatggcaaagtgcttcaaaccccagtgtattcttcaaggtgggggatacatccaggtttagtgtgccttatgtaggattggcatcagcatataattgtttttatgatggttactcacatgatgatgcagaaactcagtatggcataactgttctaaaccatatgggtagtatggcattcagaatagtaaatgaacatgatgaacataaaactcttgtcaagatcagagtttatcacagggcaaagcacgttgaagcatggattccaagagcacccagagcactaccctacacatcaatagggcgcacaaattatcctaagaatacagaaccagtaattaagaagaggaaaggtgacattaaatcctatggtttaggacctaggtacggtgggatttatacatcaaatgttaaaataatgaattaccacttgatgacaccagaagaccaccataatctgatagcaccctatccaaatagagatttagcaatagtctcaacaggaggacatggtgcagaaacaataccacactgtaactgtacatcaggtgtttactattccacatattacagaaagtattaccccataatttgtgaaaagcccaccaacatctggattgaaggaaacccttattacccaagtaggtttcaagcaggagtgatgaaaggggttgggccagcagaaccaggagactgcggtgggattttgagatgcatacatggtcccattggattgttaacagctggaggtagtggatatgtttgttttgctgacatacgacagttggagtgtatcgcagaggaacaggggctgagtgattacatcacaggtttgggtagagcttttggtgtcgggttcactgaccaaatctcaacaaaagtcacagaactacaagaagtggcgaaagatttcctcaccacaaaagttttgtccaaagtggtcaaaatggtttcagctttagtgatcatttgcagaaatcatgatgacttggtcactgttacggccactctagcactacttggatgtgatggatctccctggagatttctgaagatgtacatttccaaacactttcaggtgccttacattgaaagacaagcaaatgatggatggttcagaaagtttaatgatgcatgtaatgctgcaaagggattggaatggattgctaataagatttccaaactgattgaatggataaaaaacaaagtacttccccaagccaaagaaaaactagaattttgtagtaaactcaaacaacttgatatactagagagacaaataaccaccatgcatatctcgaatccaacacaggaaaaacgagagcagttgttcaacaacgtattgtggttggaacaaatgtcgcaaaagtttgccccacattatgccgttgaatcaaaaagaatcagggaactcaagaacaaaatggtaaattatatgcaatttaaaagtaaacaaagaactgaaccagtgtgtgtattaatccatggtacacccggttctggtaaatcattaacaacatccattgtgggacgtgcaattgcagaacacttcaattcagcagtatattcacttccaccagatcccaagcactttgatggttatcagcaacaggaagttgtgattatggatgatctgaaccaaaatccagatggacaggatataagcatgttttgtcaaatggtttcttcagtggatttcttgcctccaatggctagtttagataacaagggcatgttattcaccagtaattttgttctagcctccacaaattctaacacactaagccccccaacaatcttgaatcctgaagctttagtcaggagatttggttttgacctggatatatgtttgcatactacctacacaaagaatggaaaactcaatgcaggcatgtcaaccaagacatgcaaagattgccatcaaccatctaatttcaagaaatgttgccccctggtctgtggaaaagctattagcttggtagacagaactaccaacgttaggtatagtgtggatcaactggtcacagctattataagtgatttcaagagcaaaatgcaaattacagattccctagaaacactgtttcaaggaccagtgtataaagatttagagattgatgtttgcaacacaccacctccagaatgtatcaacgatttactgaaatctgtagattcagaagagattagggaatattgtaagaagaagaaatggattatacctgaaattcctaccaacatagaaagggctatgaatcaagccagcatgattattaatactattctgatgtttgtcagtacattaggtattgtttatgtcatttataaattgtttgctcaaactcaaggaccatattctggtaacccgcctcacaataaactaaaagccccaactttacgcccagttgttgtgcaaggaccaaacacagaatttgcactatccctgttaaggaaaaacataatgactataacaacctcaaagggagagttcacagggttaggcatacatgatcgtgtctgtgtgatacccacacacgcacagcctggtgatgatgtactagtgaatggtcagaaaattagagttaaggataagtacaaattagtagatccagagaacattaatctagagcttacagtgttgactttagatagaaatgaaaaattcagagatatcaggggatttatatcagaagatctagaaggtgtggatgccactttggtagtacattcaaataactttaccaacactatcttagaagttggccctgtaacaatggcaggacttattaatttgagtagcacccccactaacagaatgattcgttatgattatgcaacaaaaactgggcagtgtggaggtgtgctgtgtgctactggtaagatctttggtattcatgttggcggtaatggaagacaaggattttcagctcaacttaaaaaacaatattttgtagagaaacaaggccaagtaatagctagacataaggttagggagtttaacataaatccagtcaacacgccaaccaagtcaaaattacatcccagtgtattctatgatgttttcccaggtgacaaggaacctgctgtattgagtgacaatgatcccagactggaagttaaattgactgaatcattattctctaagtacaaggggaatgtaaatacggaacccactgaaaatatgcttgtggctgtagaccattatgcagggcaactattatcactagatatccccacttctgaacttacactaaaagaagcattatatggagtagatggactagaacctatagatattacaaccagtgcaggatttccctatgtgagtcttgggatcaaaaagagagacattctgaacaaagagacccaggacacagaaaagatgaagttttatctagacaagtatggcattgacttgcctctagttacatatattaaggatgaattaagaagtgttgacaaagtccgattagggaaaagtagattaattgaagcctccagtttgaatgattctgttaacatgagaatgaaactaggcaacctttacaaagcattccatcaaaatcccggtgttctgactgggtcagcagtgggttgtgatcctgatgtgttttggtctgtcatcccttgcttaatggatgggcacctgatggcatttgattactctaattttgatgcctctttgtcaccagtttggtttgtctgtctagagaaggttttgaccaagttaggctttgcaggctcttcattaattcaatcaatttgtaatacccatcatatctttagggatgaaatatatgtggttgaaggtggcatgccctcagggtgttcaggaaccagcatattcaattccatgatcaacaacataatcattaggactttgatattagatgcatataaaggaatagatttagacaaacttaaaatcttagcttacggtgatgatttgattgtttcttatccttatgaactggatccacaagtgttggcaactcttggtaaaaattatggactaaccatcacacccccagacaaatctgaaacttttacaaaaatgacatgggaaaacttgacatttttaaagagatacttcaagcctgatcaacaatttccctttttggttcacccagttatgcccatgaaagatatacatgagtcaatcagatggacaaaggatcctaaaaacacacaggatcacgtccgatcattatgcatgttagcatggcactcaggagaaaaagagtacaatgaattcattcagaagatcagaactactgacattggaaaatgtctaattctcccagaatacagcgtacttaggaggcgctggttggacctcttttaggttaacaatatagacacttaatttgagtagaagtaggagtttataaaaaaaaaaaaaaaaaaaaaaaaaaaaaaaaaaaaaaaaaaacgcgtggggatcctctagagtcgacggtaccaggaggaactatatccggattggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcaggtggcacttttcggggaaatgtgcgcggaacccctatttgtttatttttctaaatacattcaaatatgtatccgctcatgaattaattcttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcataacaccccttgtattactgtttatgtaagcagacagttttattgttcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtcgagatcccggtgcctaatgagtgagctaacttacattaattgcgttctgcagctaatacgactcactata

SEQ ID NO: 81 - protein, 23 aa, Artificial Sequence
  Synthetic Construct
SLLKEVETPIRNEWGSRSNDSSD

SEQ ID NO: 82 - protein, 882 aa, Artificial Sequence
  Synthetic construct
MGAQVSTQKSGSHENQNILTNGSNQTFTVINYYKDAASTSSAGQSLSMDPSKFTEPVKDLMLKGAPALNSPNVEACGYSDRVQQITLGNSTITTQEAANAVVCYAEWPEYLPDVDASDVNKTSKPDTSVCRFYTLDSKTWTTGSKGWCWKLPDALKDMGVFGQNMFFHSLGRSGYTVHVQCNATKFHSGCLLVVVIPEHQLASHEGGNVSVKYTFTHPGERGIDLSSAGTHSLLKEVETPIRNEWGSRSNDSSDNEVGGPVKDVIYNMNGTLLGNLLIFPHQFINLRTNNTATIVIPYINSVPIDSMTRHNNVSLMVIPIAPLTVPTGATPSLPITVTIAPMCTEFSGIRSKSIVPQGLPTTTLPGSGQFLTTDDRQSPSALPNYEPTPRIHIPGKVHNLLEIIQVDTLIPMNNTHTKDEVNSYLIPLNANRQNEQVFGTNLFIGDGVFKTTLLGEIVQYYTHWSGSLRFSLMYTGPALSSAKLILAYTPPGARGPQDRREAMLGTHVVWDIGLQSTIVMTIPWTSGVQFRYTDPDTYTSAGFLSCWYQTSLILPPETTGQVYLLSFISACPDFKLRLMKDTQTISQTVALTEGLGDELEEVIVEKTKQTVASISSGPKHTQKVPILTANETGATMPVLPSDSIETRTTYMHFNGSETDVECFLGRAACVHVTEIQNKDATGIDNHREAKLFNDWKINLSSLVQLRKKLELFTYVRFDSEYTILATASQPDSANYSSNLVVQAMYVPPGAPNPKEWDDYTWQSASNPSVFFKVGDTSRFSVPYVGLASAYNCFYDGYSHDDAETQYGITVLNHMGSMAFRIVNEHDEHKTLVKIRVYHRAKHVEAWIPRAPRALPYTSIGRTNYPKNTEPVIKKRKGDIKSY

SEQ ID NO: 83 - DNA, 10921 bp, Artificial Sequence
  Synthetic Construct
gggttaaaacagcggatgggtatcccaccattcgacccattgggtgtagtactctggtactatgtacctttgtacgcctgtttctccccaaccacccttccttaaaattcccacccatgaaacgttagaagcttgacattaaagtacaataggtggcgccatatccaatggtgtctatgtacaagcacttctgtttccccggagcgaggtataggctgtacccactgccaaaagcctttaaccgttatccgccaaccaactacgtaacagttagtaccatcttgttcttgactggacgttcgatcaggtggattttccctccactagtttggtcgatgaggctaggaattccccacgggtgaccgtgtcctagcctgcgtggcggccaacccagcttatgctgggacgcccttttaaggacatggtgtgaagactcgcatgtgcttggttgtgagtcctccggcccctgaatgcggctaaccttaaccctggagccttatgccacgatccagtggttgtaaggtcgtaatgagcaactccgggacgggaccgactactttgggtgtccgtgtttctcatttttcttcatattgtcttatggtcacagcatatatatacatatactgtgatcatgggcgctcaggtttctacacagaaaagtggatctcacgaaaatcaaaacattttgaccaatggatcaaatcagactttcacagttataaattactataaggatgcagcaagtacatcatcagctggtcaatcactgtcaatggacccatctaagtttacagaaccagttaaagatctcatgcttaagggtgcaccagcattgaattcacccaatgttgaggcctgtggttatagtgatagagtacaacaaatcacactcgggaattcaacaataacaacacaagaagcagccaacgctgttgtgtgttatgctgaatggccagagtaccttccagatgtggacgctagtgatgtcaataaaacttcaaaaccagacacttctgtctgtaggttttacacattggatagtaagacatggacaacaggttctaaaggctggtgctggaaattaccagatgcactcaaagatatgggtgtgttcgggcaaaacatgtttttccactcactaggaagatcaggttacacagtacacgttcagtgcaatgccacaaaattccatagcggttgtctacttgtagttgtaataccagaacaccaactggcttcacatgagggtggcaatgtttcagttaaatacacattcacgcatccaggtgaacgtggtatagatttatcatctgcaggcacccactcattattaaaagaagttgaaacaccaataagaaatgaatggggctcgagatcaaatgattcatcagataatgaagtgggagggcctgtcaaggatgtcatatacaatatgaatggtactttattaggaaatctgctcattttccctcaccagttcattaatctaagaaccaataatacagccacaatagtgataccatacataaactcagtacccattgattcaatgacacgtcacaacaatgtctcactgatggtcatccctattgcccctcttacagtaccaactggagcaactccctcactccctataacagtcacaatagcacctatgtgcactgagttctctgggataaggtccaagtcaattgtgccacaaggtttgccaactacaactttgccggggtcaggacaattcttgaccacagatgacaggcaatcccccagtgcactgccaaattatgagccaactccaagaatacacataccagggaaagttcataacttgctagaaattatacaggtagatacactcattcctatgaacaacacgcatacaaaagatgaggttaacagttacctcataccactaaatgcaaacaggcaaaatgagcaggtttttgggacaaacctgtttattggtgatggggtcttcaaaactactcttctgggtgaaattgttcagtactatacacattggtctggatcacttagattctctttgatgtatactggtcctgccttgtccagtgctaaactcattctagcatacaccccgcctggtgctcgtggtccacaggacaggagagaagcaatgctaggtactcatgttgtctgggatattggtctgcaatccaccatagtaatgacaataccatggacatcaggggtgcagtttagatatactgatccagatacatacaccagtgctggctttctatcatgttggtatcaaacttctcttatacttcccccagaaacgaccggccaggtctacttattatcattcataagtgcatgtccagattttaagcttaggctgatgaaagatactcaaactatctcacagactgttgcactcactgaaggcttaggtgatgaattagaagaagtcatcgttgagaaaacgaaacagacggtggcctcaatctcatctggtccaaaacacacacaaaaagtccccatactaactgcaaacgaaacaggggccacaatgcctgttcttccatcagacagcatagaaaccagaactacctacatgcactttaatggttcagaaactgatgtagaatgctttttgggtcgtgcagcttgtgtgcatgtaactgaaatacaaaacaaagatgctactggaatagataatcacagagaagcaaaattgttcaatgattggaaaatcaacctgtccagccttgtccaacttagaaagaaactagaactcttcacttatgttaggtttgattctgagtataccatactggccactgcatctcaacctgattcagcaaactattcaagcaatttggtggtccaagccatgtatgttccacctggtgccccgaatccaaaagagtgggacgattacacatggcaaagtgcttcaaaccccagtgtattcttcaaggtgggggatacatccaggtttagtgtgccttatgtaggattggcatcagcatataattgtttttatgatggttactcacatgatgatgcagaaactcagtatggcataactgttctaaaccatatgggtagtatggcattcagaatagtaaatgaacatgatgaacataaaactcttgtcaagatcagagtttatcacagggcaaagcacgttgaagcatggattccaagagcacccagagcactaccctacacatcaatagggcgcacaaattatcctaagaatacagaaccagtaattaagaagaggaaaggtgacattaaatcctatggtttaggacctaggtacggtgggatttatacatcaaatgttaaaataatgaattaccacttgatgacaccagaagaccaccataatctgatagcaccctatccaaatagagatttagcaatagtctcaacaggaggacatggtgcagaaacaataccacactgtaactgtacatcaggtgtttactattccacatattacagaaagtattaccccataatttgtgaaaagcccaccaacatctggattgaaggaaacccttattacccaagtaggtttcaagcaggagtgatgaaaggggttgggccagcagaaccaggagactgcggtgggattttgagatgcatacatggtcccattggattgttaacagctggaggtagtggatatgtttgttttgctgacatacgacagttggagtgtatcgcagaggaacaggggctgagtgattacatcacaggtttgggtagagcttttggtgtcgggttcactgaccaaatctcaacaaaagtcacagaactacaagaagtggcgaaagatttcctcaccacaaaagttttgtccaaagtggtcaaaatggtttcagctttagtgatcatttgcagaaatcatgatgacttggtcactgttacggccactctagcactacttggatgtgatggatctccctggagatttctgaagatgtacatttccaaacactttcaggtgccttacattgaaagacaagcaaatgatggatggttcagaaagtttaatgatgcatgtaatgctgcaaagggattggaatggattgctaataagatttccaaactgattgaatggataaaaaacaaagtacttccccaagccaaagaaaaactagaattttgtagtaaactcaaacaacttgatatactagagagacaaataaccaccatgcatatctcgaatccaacacaggaaaaacgagagcagttgttcaacaacgtattgtggttggaacaaatgtcgcaaaagtttgccccacattatgccgttgaatcaaaaagaatcagggaactcaagaacaaaatggtaaattatatgcaatttaaaagtaaacaaagaactgaaccagtgtgtgtattaatccatggtacacccggttctggtaaatcattaacaacatccattgtgggacgtgcaattgcagaacacttcaattcagcagtatattcacttccaccagatcccaagcactttgatggttatcagcaacaggaagttgtgattatggatgatctgaaccaaaatccagatggacaggatataagcatgttttgtcaaatggtttcttcagtggatttcttgcctccaatggctagtttagataacaagggcatgttattcaccagtaattttgttctagcctccacaaattctaacacactaagccccccaacaatcttgaatcctgaagctttagtcaggagatttggttttgacctggatatatgtttgcatactacctacacaaagaatggaaaactcaatgcaggcatgtcaaccaagacatgcaaagattgccatcaaccatctaatttcaagaaatgttgccccctggtctgtggaaaagctattagcttggtagacagaactaccaacgttaggtatagtgtggatcaactggtcacagctattataagtgatttcaagagcaaaatgcaaattacagattccctagaaacactgtttcaaggaccagtgtataaagatttagagattgatgtttgcaacacaccacctccagaatgtatcaacgatttactgaaatctgtagattcagaagagattagggaatattgtaagaagaagaaatggattatacctgaaattcctaccaacatagaaagggctatgaatcaagccagcatgattattaatactattctgatgtttgtcagtacattaggtattgtttatgtcatttataaattgtttgctcaaactcaaggaccatattctggtaacccgcctcacaataaactaaaagccccaactttacgcccagttgttgtgcaaggaccaaacacagaatttgcactatccctgttaaggaaaaacataatgactataacaacctcaaagggagagttcacagggttaggcatacatgatcgtgtctgtgtgatacccacacacgcacagcctggtgatgatgtactagtgaatggtcagaaaattagagttaaggataagtacaaattagtagatccagagaacattaatctagagcttacagtgttgactttagatagaaatgaaaaattcagagatatcaggggatttatatcagaagatctagaaggtgtggatgccactttggtagtacattcaaataactttaccaacactatcttagaagttggccctgtaacaatggcaggacttattaatttgagtagcacccccactaacagaatgattcgttatgattatgcaacaaaaactgggcagtgtggaggtgtgctgtgtgctactggtaagatctttggtattcatgttggcggtaatggaagacaaggattttcagctcaacttaaaaaacaatattttgtagagaaacaaggccaagtaatagctagacataaggttagggagtttaacataaatccagtcaacacgccaaccaagtcaaaattacatcccagtgtattctatgatgttttcccaggtgacaaggaacctgctgtattgagtgacaatgatcccagactggaagttaaattgactgaatcattattctctaagtacaaggggaatgtaaatacggaacccactgaaaatatgcttgtggctgtagaccattatgcagggcaactattatcactagatatccccacttctgaacttacactaaaagaagcattatatggagtagatggactagaacctatagatattacaaccagtgcaggatttccctatgtgagtcttgggatcaaaaagagagacattctgaacaaagagacccaggacacagaaaagatgaagttttatctagacaagtatggcattgacttgcctctagttacatatattaaggatgaattaagaagtgttgacaaagtccgattagggaaaagtagattaattgaagcctccagtttgaatgattctgttaacatgagaatgaaactaggcaacctttacaaagcattccatcaaaatcccggtgttctgactgggtcagcagtgggttgtgatcctgatgtgttttggtctgtcatcccttgcttaatggatgggcacctgatggcatttgattactctaattttgatgcctctttgtcaccagtttggtttgtctgtctagagaaggttttgaccaagttaggctttgcaggctcttcattaattcaatcaatttgtaatacccatcatatctttagggatgaaatatatgtggttgaaggtggcatgccctcagggtgttcaggaaccagcatattcaattccatgatcaacaacataatcattaggactttgatattagatgcatataaaggaatagatttagacaaacttaaaatcttagcttacggtgatgatttgattgtttcttatccttatgaactggatccacaagtgttggcaactcttggtaaaaattatggactaaccatcacacccccagacaaatctgaaacttttacaaaaatgacatgggaaaacttgacatttttaaagagatacttcaagcctgatcaacaatttccctttttggttcacccagttatgcccatgaaagatatacatgagtcaatcagatggacaaaggatcctaaaaacacacaggatcacgtccgatcattatgcatgttagcatggcactcaggagaaaaagagtacaatgaattcattcagaagatcagaactactgacattggaaaatgtctaattctcccagaatacagcgtacttaggaggcgctggttggacctcttttaggttaacaatatagacacttaatttgagtagaagtaggagtttataaaaaaaaaaaaaaaaaaaaaaaaaaaaaaaaaaaaaaaaaaacgcgtggggatcctctagagtcgacggtaccaggaggaactatatccggattggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcaggtggcacttttcggggaaatgtgcgcggaacccctatttgtttatttttctaaatacattcaaatatgtatccgctcatgaattaattcttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcataacaccccttgtattactgtttatgtaagcagacagttttattgttcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtcgagatcccggtgcctaatgagtgagctaacttacattaattgcgttctgcagctaatacgactcactata

SEQ ID NO: 84 - protein, 886 aa, Artificial Sequence
  Synthetic construct
MGAQVSTQKSGSHENQNILTNGSNQTFTVINYYKDAASTSSAGQSLSMDPSKFTEPVKDLMLKGAPALNSPNVEACGYSDRVQQITLGNSTITTQEAANAVVCYAEWPEYLPDVDASDVNKTSKPDTSVCRFYTLDSKTWTTGSKGWCWKLPDALKDMGVFGQNMFFHSLGRSGYTVHVQCNATKFHSGCLLVVVIPEHQLASHEGGNVSVKYTFTHPGERGIDLSSAQPASLLAEVETPIRNEWGSERGIFGAIAGFIEPGGPVKDVIYNMNGTLLGNLLIFPHQFINLRTNNTATIVIPYINSVPIDSMTRHNNVSLMVIPIAPLTVPTGATPSLPITVTIAPMCTEFSGIRSKSIVPQGLPTTTLPGSGQFLTTDDRQSPSALPNYEPTPRIHIPGKVHNLLEIIQVDTLIPMNNTHTKDEVNSYLIPLNANRQNEQVFGTNLFIGDGVFKTTLLGEIVQYYTHWSGSLRFSLMYTGPALSSAKLILAYTPPGARGPQDRREAMLGTHVVWDIGLQSTIVMTIPWTSGVQFRYTDPDTYTSAGFLSCWYQTSLILPPETTGQVYLLSFISACPDFKLRLMKDTQTISQTVALTEGLGDELEEVIVEKTKQTVASISSGPKHTQKVPILTANETGATMPVLPSDSIETRTTYMHFNGSETDVECFLGRAACVHVTEIQNKDATGIDNHREAKLFNDWKINLSSLVQLRKKLELFTYVRFDSEYTILATASQPDSANYSSNLVVQAMYVPPGAPNPKEWDDYTWQSASNPSVFFKVGDTSRFSVPYVGLASAYNCFYDGYSHDDAETQYGITVLNHMGSMAFRIVNEHDEHKTLVKIRVYHRAKHVEAWIPRAPRALPYTSIGRTNYPKNTEPVIKKRKGDIKSY

SEQ ID NO: 85 - DNA, 10933 bp, Artificial Sequence
  Synthetic construct
gggttaaaacagcggatgggtatcccaccattcgacccattgggtgtagtactctggtactatgtacctttgtacgcctgtttctccccaaccacccttccttaaaattcccacccatgaaacgttagaagcttgacattaaagtacaataggtggcgccatatccaatggtgtctatgtacaagcacttctgtttccccggagcgaggtataggctgtacccactgccaaaagcctttaaccgttatccgccaaccaactacgtaacagttagtaccatcttgttcttgactggacgttcgatcaggtggattttccctccactagtttggtcgatgaggctaggaattccccacgggtgaccgtgtcctagcctgcgtggcggccaacccagcttatgctgggacgcccttttaaggacatggtgtgaagactcgcatgtgcttggttgtgagtcctccggcccctgaatgcggctaaccttaaccctggagccttatgccacgatccagtggttgtaaggtcgtaatgagcaactccgggacgggaccgactactttgggtgtccgtgtttctcatttttcttcatattgtcttatggtcacagcatatatatacatatactgtgatcatgggcgctcaggtttctacacagaaaagtggatctcacgaaaatcaaaacattttgaccaatggatcaaatcagactttcacagttataaattactataaggatgcagcaagtacatcatcagctggtcaatcactgtcaatggacccatctaagtttacagaaccagttaaagatctcatgcttaagggtgcaccagcattgaattcacccaatgttgaggcctgtggttatagtgatagagtacaacaaatcacactcgggaattcaacaataacaacacaagaagcagccaacgctgttgtgtgttatgctgaatggccagagtaccttccagatgtggacgctagtgatgtcaataaaacttcaaaaccagacacttctgtctgtaggttttacacattggatagtaagacatggacaacaggttctaaaggctggtgctggaaattaccagatgcactcaaagatatgggtgtgttcgggcaaaacatgtttttccactcactaggaagatcaggttacacagtacacgttcagtgcaatgccacaaaattccatagcggttgtctacttgtagttgtaataccagaacaccaactggcttcacatgagggtggcaatgtttcagttaaatacacattcacgcatccaggtgaacgtggtatagatttatcatctgcacagcccgcatcattattagcagaagttgaaacaccaataagaaatgaatggggctctgaacgtggtatatttggtgcaatagcaggttttatagaaccgggagggcctgtcaaggatgtcatatacaatatgaatggtaccttattaggaaatctgctcattttccctcaccagttcattaatctaagaaccaataatacagccacaatagtgataccatacataaactcagtacccattgattcaatgacacgtcacaacaatgtctcactgatggtcatccctattgcccctcttacagtaccaactggagcaactccctcactccctataacagtcacaatagcacctatgtgcactgagttctctgggataaggtccaagtcaattgtgccacaaggtttgccaactacaactttgccggggtcaggacaattcttgaccacagatgacaggcaatcccccagtgcactgccaaattatgagccaactccaagaatacacataccagggaaagttcataacttgctagaaattatacaggtagatacactcattcctatgaacaacacgcatacaaaagatgaggttaacagttacctcataccactaaatgcaaacaggcaaaatgagcaggtttttgggacaaacctgtttattggtgatggggtcttcaaaactactcttctgggtgaaattgttcagtactatacacattggtctggatcacttagattctctttgatgtatactggtcctgccttgtccagtgctaaactcattctagcatacaccccgcctggtgctcgtggtccacaggacaggagagaagcaatgctaggtactcatgttgtctgggatattggtctgcaatccaccatagtaatgacaataccatggacatcaggggtgcagtttagatatactgatccagatacatacaccagtgctggctttctatcatgttggtatcaaacttctcttatacttcccccagaaacgaccggccaggtctacttattatcattcataagtgcatgtccagattttaagcttaggctgatgaaagatactcaaactatctcacagactgttgcactcactgaaggcttaggtgatgaattagaagaagtcatcgttgagaaaacgaaacagacggtggcctcaatctcatctggtccaaaacacacacaaaaagtccccatactaactgcaaacgaaacaggggccacaatgcctgttcttccatcagacagcatagaaaccagaactacctacatgcactttaatggttcagaaactgatgtagaatgctttttgggtcgtgcagcttgtgtgcatgtaactgaaatacaaaacaaagatgctactggaatagataatcacagagaagcaaaattgttcaatgattggaaaatcaacctgtccagccttgtccaacttagaaagaaactagaactcttcacttatgttaggtttgattctgagtataccatactggccactgcatctcaacctgattcagcaaactattcaagcaatttggtggtccaagccatgtatgttccacctggtgccccgaatccaaaagagtgggacgattacacatggcaaagtgcttcaaaccccagtgtattcttcaaggtgggggatacatccaggtttagtgtgccttatgtaggattggcatcagcatataattgtttttatgatggttactcacatgatgatgcagaaactcagtatggcataactgttctaaaccatatgggtagtatggcattcagaatagtaaatgaacatgatgaacataaaactcttgtcaagatcagagtttatcacagggcaaagcacgttgaagcatggattccaagagcacccagagcactaccctacacatcaatagggcgcacaaattatcctaagaatacagaaccagtaattaagaagaggaaaggtgacattaaatcctatggtttaggacctaggtacggtgggatttatacatcaaatgttaaaataatgaattaccacttgatgacaccagaagaccaccataatctgatagcaccctatccaaatagagatttagcaatagtctcaacaggaggacatggtgcagaaacaataccacactgtaactgtacatcaggtgtttactattccacatattacagaaagtattaccccataatttgtgaaaagcccaccaacatctggattgaaggaaacccttattacccaagtaggtttcaagcaggagtgatgaaaggggttgggccagcagaaccaggagactgcggtgggattttgagatgcatacatggtcccattggattgttaacagctggaggtagtggatatgtttgttttgctgacatacgacagttggagtgtatcgcagaggaacaggggctgagtgattacatcacaggtttgggtagagcttttggtgtcgggttcactgaccaaatctcaacaaaagtcacagaactacaagaagtggcgaaagatttcctcaccacaaaagttttgtccaaagtggtcaaaatggtttcagctttagtgatcatttgcagaaatcatgatgacttggtcactgttacggccactctagcactacttggatgtgatggatctccctggagatttctgaagatgtacatttccaaacactttcaggtgccttacattgaaagacaagcaaatgatggatggttcagaaagtttaatgatgcatgtaatgctgcaaagggattggaatggattgctaataagatttccaaactgattgaatggataaaaaacaaagtacttccccaagccaaagaaaaactagaattttgtagtaaactcaaacaacttgatatactagagagacaaataaccaccatgcatatctcgaatccaacacaggaaaaacgagagcagttgttcaacaacgtattgtggttggaacaaatgtcgcaaaagtttgccccacattatgccgttgaatcaaaaagaatcagggaactcaagaacaaaatggtaaattatatgcaatttaaaagtaaacaaagaactgaaccagtgtgtgtattaatccatggtacacccggttctggtaaatcattaacaacatccattgtgggacgtgcaattgcagaacacttcaattcagcagtatattcacttccaccagatcccaagcactttgatggttatcagcaacaggaagttgtgattatggatgatctgaaccaaaatccagatggacaggatataagcatgttttgtcaaatggtttcttcagtggatttcttgcctccaatggctagtttagataacaagggcatgttattcaccagtaattttgttctagcctccacaaattctaacacactaagccccccaacaatcttgaatcctgaagctttagtcaggagatttggttttgacctggatatatgtttgcatactacctacacaaagaatggaaaactcaatgcaggcatgtcaaccaagacatgcaaagattgccatcaaccatctaatttcaagaaatgttgccccctggtctgtggaaaagctattagcttggtagacagaactaccaacgttaggtatagtgtggatcaactggtcacagctattataagtgatttcaagagcaaaatgcaaattacagattccctagaaacactgtttcaaggaccagtgtataaagatttagagattgatgtttgcaacacaccacctccagaatgtatcaacgatttactgaaatctgtagattcagaagagattagggaatattgtaagaagaagaaatggattatacctgaaattcctaccaacatagaaagggctatgaatcaagccagcatgattattaatactattctgatgtttgtcagtacattaggtattgtttatgtcatttataaattgtttgctcaaactcaaggaccatattctggtaacccgcctcacaataaactaaaagccccaactttacgcccagttgttgtgcaaggaccaaacacagaatttgcactatccctgttaaggaaaaacataatgactataacaacctcaaagggagagttcacagggttaggcatacatgatcgtgtctgtgtgatacccacacacgcacagcctggtgatgatgtactagtgaatggtcagaaaattagagttaaggataagtacaaattagtagatccagagaacattaatctagagcttacagtgttgactttagatagaaatgaaaaattcagagatatcaggggatttatatcagaagatctagaaggtgtggatgccactttggtagtacattcaaataactttaccaacactatcttagaagttggccctgtaacaatggcaggacttattaatttgagtagcacccccactaacagaatgattcgttatgattatgcaacaaaaactgggcagtgtggaggtgtgctgtgtgctactggtaagatctttggtattcatgttggcggtaatggaagacaaggattttcagctcaacttaaaaaacaatattttgtagagaaacaaggccaagtaatagctagacataaggttagggagtttaacataaatccagtcaacacgccaaccaagtcaaaattacatcccagtgtattctatgatgttttcccaggtgacaaggaacctgctgtattgagtgacaatgatcccagactggaagttaaattgactgaatcattattctctaagtacaaggggaatgtaaatacggaacccactgaaaatatgcttgtggctgtagaccattatgcagggcaactattatcactagatatccccacttctgaacttacactaaaagaagcattatatggagtagatggactagaacctatagatattacaaccagtgcaggatttccctatgtgagtcttgggatcaaaaagagagacattctgaacaaagagacccaggacacagaaaagatgaagttttatctagacaagtatggcattgacttgcctctagttacatatattaaggatgaattaagaagtgttgacaaagtccgattagggaaaagtagattaattgaagcctccagtttgaatgattctgttaacatgagaatgaaactaggcaacctttacaaagcattccatcaaaatcccggtgttctgactgggtcagcagtgggttgtgatcctgatgtgttttggtctgtcatcccttgcttaatggatgggcacctgatggcatttgattactctaattttgatgcctctttgtcaccagtttggtttgtctgtctagagaaggttttgaccaagttaggctttgcaggctcttcattaattcaatcaatttgtaatacccatcatatctttagggatgaaatatatgtggttgaaggtggcatgccctcagggtgttcaggaaccagcatattcaattccatgatcaacaacataatcattaggactttgatattagatgcatataaaggaatagatttagacaaacttaaaatcttagcttacggtgatgatttgattgtttcttatccttatgaactggatccacaagtgttggcaactcttggtaaaaattatggactaaccatcacacccccagacaaatctgaaacttttacaaaaatgacatgggaaaacttgacatttttaaagagatacttcaagcctgatcaacaatttccctttttggttcacccagttatgcccatgaaagatatacatgagtcaatcagatggacaaaggatcctaaaaacacacaggatcacgtccgatcattatgcatgttagcatggcactcaggagaaaaagagtacaatgaattcattcagaagatcagaactactgacattggaaaatgtctaattctcccagaatacagcgtacttaggaggcgctggttggacctcttttaggttaacaatatagacacttaatttgagtagaagtaggagtttataaaaaaaaaaaaaaaaaaaaaaaaaaaaaaaaaaaaaaaaaaacgcgtggggatcctctagagtcgacggtaccaggaggaactatatccggattggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcaggtggcacttttcggggaaatgtgcgcggaacccctatttgtttatttttctaaatacattcaaatatgtatccgctcatgaattaattcttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcataacaccccttgtattactgtttatgtaagcagacagttttattgttcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtcgagatcccggtgcctaatgagtgagctaacttacattaattgcgttctgcagctaatacgactcactata

SEQ ID NO: 86 - DNA, 2628 bp, Artificial Sequence
  Synthetic Construct
atgggcgctcaggtttctacacagaaaagtggatctcacgaaaatcaaaacattttgaccaatggatcaaatcagactttcacagttataaattactataaggatgcagcaagtacatcatcagctggtcaatcactgtcaatggacccatctaagtttacagaaccagttaaagatctcatgcttaagggtgcaccagcattgaattcacccaatgttgaggcctgtggttatagtgatagagtacaacaaatcacactcgggaattcaacaataacaacacaagaagcagccaacgctgttgtgtgttatgctgaatggccagagtaccttccagatgtggacgctagtgatgtcaataaaacttcaaaaccagacacttctgtctgtaggttttacacattggatagtaagacatggacaacaggttctaaaggctggtgctggaaattaccagatgcactcaaagatatgggtgtgttcgggcaaaacatgtttttccactcactaggaagatcaggttacacagtacacgttcagtgcaatgccacaaaattccatagcggttgtctacttgtagttgtaataccagaacaccaactggcttcacatgagggtggcaatgtttcagttaaatacacattcacgcatccaggtgaacgtggtatagatttatcatctgcacagcccgcatcattattaacagaagttgaaacaccaataagaaatgaatggggctcgagaaatgaagtgggagggcctgtcaaggatgtcatatacaatatgaatggtactttattaggaaatctgctcattttccctcaccagttcattaatctaagaaccaataatacagccacaatagtgataccatacataaactcagtacccattgattcaatgacacgtcacaacaatgtctcactgatggtcatccctattgcccctcttacagtaccaactggagcaactccctcactccctataacagtcacaatagcacctatgtgcactgagttctctgggataaggtccaagtcaattgtgccacaaggtttgccaactacaactttgccggggtcaggacaattcttgaccacagatgacaggcaatcccccagtgcactgccaaattatgagccaactccaagaatacacataccagggaaagttcataacttgctagaaattatacaggtagatacactcattcctatgaacaacacgcatacaaaagatgaggttaacagttacctcataccactaaatgcaaacaggcaaaatgagcaggtttttgggacaaacctgtttattggtgatggggtcttcaaaactactcttctgggtgaaattgttcagtactatacacattggtctggatcacttagattctctttgatgtatactggtcctgccttgtccagtgctaaactcattctagcatacaccccgcctggtgctcgtggtccacaggacaggagagaagcaatgctaggtactcatgttgtctgggatattggtctgcaatccaccatagtaatgacaataccatggacatcaggggtgcagtttagatatactgatccagatacatacaccagtgctggctttctatcatgttggtatcaaacttctcttatacttcccccagaaacgaccggccaggtctacttattatcattcataagtgcatgtccagattttaagcttaggctgatgaaagatactcaaactatctcacagactgttgcactcactgaaggcttaggtgatgaattagaagaagtcatcgttgagaaaacgaaacagacggtggcctcaatctcatctggtccaaaacacacacaaaaagtccccatactaactgcaaacgaaacaggggccacaatgcctgttcttccatcagacagcatagaaaccagaactacctacatgcactttaatggttcagaaactgatgtagaatgctttttgggtcgtgcagcttgtgtgcatgtaactgaaatacaaaacaaagatgctactggaatagataatcacagagaagcaaaattgttcaatgattggaaaatcaacctgtccagccttgtccaacttagaaagaaactagaactcttcacttatgttaggtttgattctgagtataccatactggccactgcatctcaacctgattcagcaaactattcaagcaatttggtggtccaagccatgtatgttccacctggtgccccgaatccaaaagagtgggacgattacacatggcaaagtgcttcaaaccccagtgtattcttcaaggtgggggatacatccaggtttagtgtgccttatgtaggattggcatcagcatataattgtttttatgatggttactcacatgatgatgcagaaactcagtatggcataactgttctaaaccatatgggtagtatggcattcagaatagtaaatgaacatgatgaacataaaactcttgtcaagatcagagtttatcacagggcaaagcacgttgaagcatggattccaagagcacccagagcactaccctacacatcaatagggcgcacaaattatcctaagaatacagaaccagtaattaagaagaggaaaggtgacattaaatcctat

SEQ ID NO: 87 - DNA, 2646 bp, Artificial Sequence
  Synthetic construct
atgggcgctcaggtttctacacagaaaagtggatctcacgaaaatcaaaacattttgaccaatggatcaaatcagactttcacagttataaattactataaggatgcagcaagtacatcatcagctggtcaatcactgtcaatggacccatctaagtttacagaaccagttaaagatctcatgcttaagggtgcaccagcattgaattcacccaatgttgaggcctgtggttatagtgatagagtacaacaaatcacactcgggaattcaacaataacaacacaagaagcagccaacgctgttgtgtgttatgctgaatggccagagtaccttccagatgtggacgctagtgatgtcaataaaacttcaaaaccagacacttctgtctgtaggttttacacattggatagtaagacatggacaacaggttctaaaggctggtgctggaaattaccagatgcactcaaagatatgggtgtgttcgggcaaaacatgtttttccactcactaggaagatcaggttacacagtacacgttcagtgcaatgccacaaaattccatagcggttgtctacttgtagttgtaataccagaacaccaactggcttcacatgagggtggcaatgtttcagttaaatacacattcacgcatccaggtgaacgtggtatagatttatcatctgcaggcacccactcattattaaaagaagttgaaacaccaataagaaatgaatggggctcgagatcaaatgattcatcagataatgaagtgggagggcctgtcaaggatgtcatatacaatatgaatggtactttattaggaaatctgctcattttccctcaccagttcattaatctaagaaccaataatacagccacaatagtgataccatacataaactcagtacccattgattcaatgacacgtcacaacaatgtctcactgatggtcatccctattgcccctcttacagtaccaactggagcaactccctcactccctataacagtcacaatagcacctatgtgcactgagttctctgggataaggtccaagtcaattgtgccacaaggtttgccaactacaactttgccggggtcaggacaattcttgaccacagatgacaggcaatcccccagtgcactgccaaattatgagccaactccaagaatacacataccagggaaagttcataacttgctagaaattatacaggtagatacactcattcctatgaacaacacgcatacaaaagatgaggttaacagttacctcataccactaaatgcaaacaggcaaaatgagcaggtttttgggacaaacctgtttattggtgatggggtcttcaaaactactcttctgggtgaaattgttcagtactatacacattggtctggatcacttagattctctttgatgtatactggtcctgccttgtccagtgctaaactcattctagcatacaccccgcctggtgctcgtggtccacaggacaggagagaagcaatgctaggtactcatgttgtctgggatattggtctgcaatccaccatagtaatgacaataccatggacatcaggggtgcagtttagatatactgatccagatacatacaccagtgctggctttctatcatgttggtatcaaacttctcttatacttcccccagaaacgaccggccaggtctacttattatcattcataagtgcatgtccagattttaagcttaggctgatgaaagatactcaaactatctcacagactgttgcactcactgaaggcttaggtgatgaattagaagaagtcatcgttgagaaaacgaaacagacggtggcctcaatctcatctggtccaaaacacacacaaaaagtccccatactaactgcaaacgaaacaggggccacaatgcctgttcttccatcagacagcatagaaaccagaactacctacatgcactttaatggttcagaaactgatgtagaatgctttttgggtcgtgcagcttgtgtgcatgtaactgaaatacaaaacaaagatgctactggaatagataatcacagagaagcaaaattgttcaatgattggaaaatcaacctgtccagccttgtccaacttagaaagaaactagaactcttcacttatgttaggtttgattctgagtataccatactggccactgcatctcaacctgattcagcaaactattcaagcaatttggtggtccaagccatgtatgttccacctggtgccccgaatccaaaagagtgggacgattacacatggcaaagtgcttcaaaccccagtgtattcttcaaggtgggggatacatccaggtttagtgtgccttatgtaggattggcatcagcatataattgtttttatgatggttactcacatgatgatgcagaaactcagtatggcataactgttctaaaccatatgggtagtatggcattcagaatagtaaatgaacatgatgaacataaaactcttgtcaagatcagagtttatcacagggcaaagcacgttgaagcatggattccaagagcacccagagcactaccctacacatcaatagggcgcacaaattatcctaagaatacagaaccagtaattaagaagaggaaaggtgacattaaatcctat